(19) United States
(12) Patent Application Publication
(10) Pub. No.: US 2006/0030814 A1
(43) Pub. Date: Feb. 9, 2006

(54) Method and apparatus for selective drug infusion via an intra-aortic flow diverter delivery catheter

(75) Inventors: Aurelio Valencia, East Palo Alto, CA (US); Ricardo Aboytes, East Palo Alto, CA (US); Jeffrey M. Elkins, Novato, CA (US); Harry B. Goodson, Fremont, CA (US); Samir R. Patel, Mountain View, CA (US); Craig A. Ball, San Carlos, CA (US); Trevor M. Greenan, Santa Rosa, CA (US); Randy J. Kesten, Mountain View, CA (US); Andrew K. Kramer, Los Gatos, CA (US); Sam G. Payne, Santa Clara, CA (US); Sophia Pesotchinsky, Los Altos Hills, CA (US); Michael H. Rosenthal, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: FlowMedica, Inc., Fremont, CA (US)

(21) Appl. No.: 11/083,802

(22) Filed: Mar. 18, 2005

Related U.S. Application Data
(63) Continuation of application No. PCT/US02/29743, filed on Sep. 20, 2002.
(60) Provisional application No. 60/412,343, filed on Sep. 20, 2002.
(60) Provisional application No. 60/412,476, filed on Sep. 20, 2002.

(51) Int. Cl. A61M 31/00

(52) U.S. Cl. 604/93.01; 606/191

(57) ABSTRACT

A local renal delivery system includes a flow isolation assembly and a local injection assembly. The flow isolation assembly in one mode is adapted to isolate only a partial flow region along the outer circumference along the aorta wall such that fluids inject there are maintained to flow substantially into the renal arteries. Various types of flow isolation assemblies and local injection assemblies are described.

### CROSS-REFERENCES TO RELATED APPLICATIONS

The present application is a continuation of PCT/US02/29743 (Attorney Docket No. 022352-001200PC) filed Sep. 22, 2003, which claims priority from U.S. provisional application Ser. Nos. 60/412,343 (Attorney Docket No. 022352-000700US), filed on Sep. 20, 2002; 60/412,476 (Attorney Docket No. 022352-000800US), filed on Sep. 20, 2002; and 60/486,349 (Attorney Docket No. 022352-001200US), filed on Jul. 10, 2003. The full disclosure of each of the foregoing applications is hereby incorporated reference.

### BACKGROUND OF THE INVENTION

1. Field of the Invention

This invention relates to the field of medical devices, and more particularly to a system and method for locally delivering fluids or agents within the body of a patient. Still more particularly, it relates to a system and method for locally delivering fluids or agents into branch blood vessels or body lumens from a main vessel or lumen, respectively, and in particular into renal arteries extending from an aorta in a patient.

2. Description of Related Art

Many different medical device systems and methods have been previously disclosed for locally delivering fluids or other agents into various body regions, including body lumens such as vessels, or other body spaces such as organs or heart chambers. Local “fluid” delivery systems may include drugs or other agents, or may even include locally delivering the body's own fluids, such as artificially enhanced blood transport (e.g. either entirely within the body such as directing or shunting blood from one place to another, or in extracorporeal modes such as via external blood pumps etc.). Local “agent” delivery systems are herein generally intended to relate to introduction of a foreign composition as an agent into the body, which may include drug or other useful or active agent, and may be in a fluid form or other form such as gels, solids, powders, gases, etc. It is to be understood that reference to only one of the terms fluid, drug, or agent with respect to local delivery descriptions may be made variously in this disclosure for illustrative purposes, but is not generally intended to be exclusive or omissive of the others; they are to be considered interchangeable where appropriate according to one of ordinary skill unless specifically described to be otherwise.

In general, local agent delivery systems and methods are often used for the benefit of achieving relatively high, localized concentrations of agent where injected within the body in order to maximize the intended effects there and while minimizing unintended peripheral effects of the agent elsewhere in the body. Where a particular dose of a locally delivered agent may be efficacious for an intended local effect, the same dose systemically delivered would be substantially diluted throughout the body before reaching the same location. The agent's intended local effect is equally diluted and efficacy is compromised. Thus systemic agent delivery requires higher dosing to achieve the required localized dose for efficacy, often resulting in compromised safety due to for example systemic reactions or side effects of the agent as it is delivered and processed elsewhere throughout the body other than at the intended target.

Various diagnostic systems and procedures have been developed using local delivery of dye (e.g. radiopaque “contrast” agent) or other diagnostic agents, wherein an external monitoring system is able to gather important physiological information based upon the diagnostic agent's movement or assimilation in the body at the location of delivery and/or at other locations affected by the delivery site. Angiography is one such practice using a hollow, tubular angiography catheter for locally injecting radiopaque dye into a blood chamber or vessel, such as for example coronary arteries in the case of coronary angiography, or in a ventricle in the case of cardiac ventriculography.

Other systems and methods have been disclosed for locally delivering therapeutic agent into a particular body tissue within a patient via a body lumen. For example, angiographic catheters of the type just described above, and other similar tubular delivery catheters, have also been disclosed for use in locally injecting treatment agents through their delivery lumens into such body spaces within the body. More detailed examples of this type include local delivery of thrombolytic drugs such as TPA™, heparin, cumadin, or urokinase into areas of existing clot or thrombogenic implants or vascular injury. In addition, various balloon catheter systems have also been disclosed for local administration of therapeutic agents into target body lumens or spaces, and in particular associated with blood vessels. More specific previously disclosed of this type include balloons with porous or perforated walls that elute drug agents through the balloon wall and into surrounding tissue such as blood vessel walls. Yet further examples for localized delivery of therapeutic agents include various multiple balloon catheters that have spaced balloons that are inflated to engage a lumen or vessel wall in order to isolate the intermediate catheter region from in-flow or out-flow across the balloons. According to these examples, a fluid agent delivery system is often coupled to this intermediate region in order to fill the region with agent such as drug that provides an intended effect at the isolated region between the balloons.

The diagnosis or treatment of many different types of medical conditions associated with various different systems, organs, and tissues, may also benefit from the ability to locally deliver fluids or agents in a controlled manner. In particular, various conditions related to the renal system would benefit a great deal from an ability to locally deliver of therapeutic, prophylactic, or diagnostic agents into the renal arteries.

Acute renal failure (“ARF”) is an abrupt decrease in the kidney's ability to excrete waste from a patient's blood. This change in kidney function may be attributable to many causes. A traumatic event, such as hemorrhage, gastrointestinal fluid loss, or renal fluid loss without proper fluid replacement may cause the patient to go into ARF. Patients may also become vulnerable to ARF after receiving anesthesia, surgery, or a-adrenergic agonists because of related systemic or renal vasoconstriction. Additionally, systemic vasodilation caused by anaphylaxis, and anti-hypertensive drugs, sepsis or drug overdose may also cause ARF because the body's natural defense is to shut down, i.e., vasoconstrict, non-essential organs such as the kidneys. Reduced cardiac output caused by cardiogenic shock, congestive heart failure, pericardial tamponade or massive pulmonary embolism creates an excess of fluid in the body, which can exacerbate congestive heart failure. For example, a reduction in blood flow and blood pressure in the kidneys due to reduced cardiac output can in turn result in the retention of excess fluid in the patient's body, leading, for example, to pulmonary and systemic edema.

Previously known methods of treating ARF, or of treating acute renal insufficiency associated with congestive heart failure (“CHF”), involve administering drugs. Examples of such drugs that have been used for this purpose include, without limitation: vasodilators, including for example papavarine, fenoldopam mesylate, calcium-channel blockers, atrial natriuretic peptide (ANP), acetylcholine, nifedipine, nitroglycerine, nitroprusside, adenosine, dopamine, and theophylline; antioxidants, such as for example acetylcysteine; and diuretics, such as for example mannitol, or furosemide. However, many of these drugs, when administered in systemic doses, have undesirable side effects. Additionally, many of these drugs would not be helpful in treating other causes of ARF. While a septic shock patient with profound systemic vasodilation often has concomitant severe renal vasoconstriction, administering vasodilators to dilate the renal artery to a patient suffering from systemic vasodilation would compound the vasodilation system wide. In addition, for patients with severe CHF (e.g., those awaiting heart transplant), mechanical methods, such as hemodialysis or left ventricular assist devices, may be implemented. Surgical device interventions, such as hemodialysis, however, generally have not been observed to be highly efficacious for long-term management of CHF. Such interventions would also not be appropriate for many patients with strong hearts suffering from ARF.

The renal system in many patients may also suffer from a particular fragility, or otherwise general exposure, to potentially harmful effects of other medical device interventions. For example, the kidneys as one of the body's main blood filtering tools may suffer damage from exposed to high density radiopaque contrast dye, such as during coronary, cardiac, or neuro angiography procedures. One particularly harmful condition known as “radiocontrast nephropathy” or “RCN” is often observed during such procedures, wherein an acute impairment of renal function follows exposure to such radiographic contrast materials, typically resulting in a rise in serum creatinine levels of more than 25% above baseline, or an absolute rise of 0.5 mg/dl within 48 hours. Therefore, in addition to CHF, renal damage associated with RCN is also a frequently observed cause of ARF. In addition, the kidneys' function is directly related to cardiac output and related blood pressure into the renal system. These physiological parameters, as in the case of CHF, may also be significantly compromised during a surgical intervention such as an angioplasty, coronary artery bypass, valve repair or replacement, or other cardiac interventional procedure. Therefore, the various drugs used to treat patients experiencing ARF associated with other conditions such as CHF have also been used to treat patients afflicted with ARF as a result of RCN. Such drugs would also provide substantial benefit for treating or preventing ARF associated with acutely compromised hemodynamics to the renal system, such as during surgical interventions.

There would be great advantage therefore from an ability to locally deliver such drugs into the renal arteries, in particular when delivered contemporaneous with surgical interventions, and in particular contemporaneous with radiocontrast dye delivery. However, many such procedures are done with medical device systems, such as using guiding catheters or angiography catheters having outer dimensions typically ranging between about 4 French to about 12 French, and ranging generally between about 6 French to about 8 French in the case of guide catheter systems for delivering angioplasty or stent devices into the coronary or neurovascular arteries (e.g. carotid arteries). These devices also are most typically delivered to their respective locations for use (e.g. coronary ostia) via a percutaneous, translumenal access in the femoral arteries and retrograde delivery upstream along the aorta past the region of the renal artery ostia. A Seldinger access technique to the femoral artery involves relatively controlled dilation of a puncture hole to minimize the size of the intruding window through the artery wall, and is a preferred method where the profiles of such delivery systems are sufficiently small. Otherwise, for larger systems a “cut-down” technique is used involving a larger, surgically made access window through the artery wall.

Accordingly, a local renal agent delivery system for contemporaneous use with other retrogradedly delivered medical device systems, such as of the types just described above, would preferably be adapted to allow for such interventional device systems, in particular of the types and dimensions just described, to pass upstream across the renal artery ostia (a) while the agent is being locally delivered into the renal arteries, and (b) while allowing blood to flow downstream across the renal artery ostia, and (c) in an overall cooperating system that allows for Seldinger femoral artery access. Each one of these features (a), (b), or (c), or any sub-combination thereof, would provide significant value to patient treatment; a local renal delivery system providing for the combination of all three features is so much the more valuable.

Notwithstanding the clear needs for and benefits that would be gained from such local drug delivery into the renal system, the ability to do so presents unique challenges as follows.

In one regard, the renal arteries extend from respective ostia along the abdominal aorta that are significantly spaced apart from each other circumferentially around the relatively very large aorta. Often, these renal artery ostia are also spaced from each other longitudinally along the aorta with relative superior and inferior locations. This presents a unique challenge to locally deliver drugs or other agents into the renal system on the whole, which requires both kidneys to be fed through these separate respective arteries via their uniquely positioned and substantially spaced apart ostia. This becomes particularly important where both kidneys may be equally at risk, or are equally compromised, during an invasive upstream procedure—or, of course, for any other indication where both kidneys require local drug delivery. Thus, an appropriate local renal delivery system for such indications would preferably be adapted to feed multiple renal arteries perfusing both kidneys.

In another regard, mere local delivery of an agent into the natural, physiologic blood flow path of the aorta upstream of the kidneys may provide some beneficial, localized renal delivery versus other systemic delivery methods, but various undesirable results still arise. In particular, the high flow aorta immediately washes much of the delivered agent beyond the intended renal artery ostia. This reduces the amount of agent actually perfusing the renal arteries with reduced efficacy, and thus also produces unwanted loss of the agent into other organs and tissues in the systemic circulation (with highest concentrations directly flowing into downstream circulation).

In still a further regard, various known types of tubular local delivery catheters, such as angiographic catheters, other “end-hole” catheters, or otherwise, may be positioned with their distal agent perfusion ports located within the renal arteries themselves for delivering agents there, such as via a percutaneous translumenal procedure via the femoral arteries (or from other access points such as brachial arteries, etc.). However, such a technique may also provide less than completely desirable results.

For example, such seating of the delivery catheter distal tip within a renal artery may be difficult to achieve from within the large diameter/high flow aorta, and may produce harmful intimal injury within the artery. Also, where multiple kidneys must be infused with agent, multiple renal arteries must be cannulated, either sequentially with a single delivery device, or simultaneously with multiple devices. This can become unnecessarily complicated and time consuming and further compound the risk of unwanted injury from the required catheter manipulation. Moreover, multiple dye injections may be required in order to locate the renal ostia for such catheter positioning, increasing the risks associated with contrast agents on kidney function (e.g. RCN)—the very organ system to be protected by the agent delivery system in the first place. Still further, the renal arteries themselves, possibly including their ostia, may have pre-existing conditions that either prevent the ability to provide the required catheter seating, or that increase the risks associated with such mechanical intrusion. For example, the artery wall may be diseased or stenotic, such as due to atherosclerotic plaque, clot, dissection, or other injury or condition. Finally, among other additional considerations, previous disclosures have yet to describe an efficacious and safe system and method for positioning these types of local agent delivery devices at the renal arteries through a common introducer or guide sheath shared with additional medical devices used for upstream interventions, such as angiography or guide catheters. In particular, to do so concurrently with multiple delivery catheters for simultaneous infusion of multiple renal arteries would further require a guide sheath of such significant dimensions that the preferred Seldinger vascular access technique would likely not be available, instead requiring the less desirable “cut-down” technique.

In addition to the various needs for locally delivering agents into branch arteries described above, much benefit may also be gained from simply locally enhancing blood perfusion into such branches, such as by increasing the blood pressure at their ostia. In particular, such enhancement would improve a number of medical conditions related to insufficient physiological perfusion into branch vessels, and in particular from an aorta and into its branch vessels such as the renal arteries.

Certain prior disclosures have provided surgical device assemblies and methods intended to enhance blood delivery into branch arteries extending from an aorta. For example, intra-aortic balloon pumps (IABPs) have been disclosed for use in diverting blood flow into certain branch arteries. One such technique involves placing an IABP in the abdominal aorta so that the balloon is situated slightly below (proximal to) the branch arteries. The balloon is selectively inflated and deflated in a counterpulsation mode (by reference to the physiologic pressure cycle) so that increased pressure distal to the balloon directs a greater portion of blood flow into principally the branch arteries in the region of their ostia. However, the flow to lower extremities downstream from such balloon system can be severely occluded during portions of this counterpulsing cycle. Moreover, such previously disclosed systems generally lack the ability to deliver drug or agent to the branch arteries while allowing continuous and substantial downstream perfusion sufficient to prevent unwanted ischemia.

It is further noted that, despite the renal risks described in relation to radiocontrast dye delivery, and in particular RCN, in certain circumstances local delivery of such dye or other diagnostic agents is indicated specifically for diagnosing the renal arteries themselves. For example, diagnosis and treatment of renal stenosis, such as due to atherosclerosis or dissection, may require dye injection into a subject renal artery. In such circumstances, enhancing the localization of the dye into the renal arteries may also be desirable. In one regard, without such localization larger volumes of dye may be required, and the dye lost into the downstream aortic flow may still be additive to impacting the kidney(s) as it circulates back there through the system. In another regard, an ability to locally deliver such dye into the renal artery from within the artery itself, such as by seating an angiography catheter there, may also be hindered by the same stenotic condition requiring the dye injection in the first place (as introduced above). Still further, patients may have stent-grafts that may prevent delivery catheter seating.

Notwithstanding the interest and advances toward locally delivering agents for treatment or diagnosis of organs or tissues, the previously disclosed systems and methods summarized immediately above generally lack the ability to effectively deliver agents from within a main artery and locally into substantially only branch arteries extending therefrom while allowing the passage of substantial blood flow and/or other medical devices through the main artery past the branches. This is in particular the case with previously disclosed renal treatment and diagnostic devices and methods, which do not adequately provide for local delivery of agents into the renal system from a location within the aorta while allowing substantial blood flow continuously downstream past the renal ostia and/or while allowing distal medical device assemblies to be passed retrogradedly across the renal ostia for upstream use. Much benefit would be gained if agents, such as protective or therapeutic drugs or radiopaque contrast dye, could be delivered to one or both of the renal arteries in such a manner.

Several more recently disclosed advances have included local flow assemblies using tubular members of varied diameters that divide flow within an aorta adjacent to renal artery ostia into outer and inner flow paths substantially perfusing the renal artery ostia and downstream circulation, respectively. Such disclosures further include delivering fluid agent primarily into the outer flow path for substantially localized delivery into the renal artery ostia. These disclosed systems and methods represent exciting new developments toward localized diagnosis and treatment of pre-existing conditions associated with branch vessels from main vessels in general, and with respect to renal arteries extending from abdominal aortas in particular.

However, such previously disclosed designs would still benefit from further modifications and improvements in order to: maximize mixing of a fluid agent within the entire circumference of the exterior flow path surrounding the tubular flow divider and perfusing multiple renal artery ostia; use the systems and methods for prophylaxis and protection of the renal system from harm, in particular during upstream interventional procedures; maximize the range of useful sizing for specific devices to accommodate a wide range of anatomic dimensions between patients; and optimize the construction, design, and inter-cooperation between system components for efficient, atraumatic use.

A need still exists for improved devices and methods for diverting blood flow principally into the renal arteries of a patient from a location within the patient's aorta adjacent the renal artery ostia along the aorta wall while at least a portion of aortic blood flow is allowed to perfuse downstream across the location of the renal artery ostia and into the patient's lower extremities.

A need still exists for improved devices and methods for substantially isolating first and second portions of aortic blood flow at a location within the aorta of a patient adjacent the renal artery ostia along the aorta wall, and directing the first portion into the renal arteries from the location within the aorta while allowing the second portion to flow across the location and downstream of the renal artery ostia into the patient's lower extremities. There is a further benefit and need for providing passive blood flow along the isolated paths and without providing active in-situ mechanical flow support to either or both of the first or second portions of aortic blood flow. Moreover, there is a further need to direct the first portion of blood along the first flow path in a manner that increases the pressure at the renal artery ostia.

A need still exists for improved devices and methods for delivering agents such as radiopaque dye or drugs into a renal artery from a location within the aorta of a patient adjacent the renal artery's ostium along the aorta wall, and without requiring translumenal positioning of an agent delivery device within the renal artery itself or its ostium.

A need still exists for improved devices and methods for locally isolating delivery of fluids or agents such as radiopaque dye or drugs simultaneously into multiple renal arteries feeding both kidneys of a patient using a single delivery device and without requiring translumenal positioning of multiple agent delivery devices respectively within the multiple renal arteries themselves.

A need still exists for improved devices and methods for locally isolating delivery of fluids or agents into the renal arteries of a patient from a location within the patient's aorta adjacent the renal artery ostia along the aorta wall, and while allowing other treatment or diagnostic devices and systems, such as angiographic or guiding catheter devices and related systems, to be delivered across the location.

A need still exists for improved devices and methods for locally delivering fluids or agents into the renal arteries from a location within the aorta of a patient adjacent to the renal artery ostia along the aorta wall, and other than as a remedial measure to treat pre-existing renal conditions, and in particular for prophylaxis or diagnostic procedures related to the kidneys.

A need still exists for improved devices and methods for locally isolating delivery of fluids or agents into the renal arteries of a patient in order to treat, protect, or diagnose the renal system adjunctive to performing other contemporaneous medical procedures such as angiograms other translumenal procedures upstream of the renal artery ostia.

A need still exists for improved devices and methods for delivering both a flow diverter system and at least one adjunctive distal interventional device, such as an angiographic or guiding catheter, through a common delivery sheath.

A need also still exists for improved devices and methods for delivering both a flow diverter system and at least one adjunctive distal interventional device, such as an angiographic or guiding catheter, through a single access site, such as a single femoral arterial puncture.

A need also still exists for improved devices and methods for treating, and in particular preventing, ARF, and in particular relation to RCN or CHF, by locally delivering renal protective or ameliorative drugs into the renal arteries, such as contemporaneous with radiocontrast injections such as during angiography procedures.

In addition to these particular needs for diverting blood flow into a patient's renal arteries via their ostia along the aorta, other similar needs also exist for diverting blood flow into other branch vessels or lumens extending from other main vessels or lumens, respectively, in a patient.

### BRIEF SUMMARY OF THE INVENTION

In general, various of the aspects of the invention described immediately below provide a local renal infusion system for treating a renal system in a patient from a location within the abdominal aorta associated with abdominal aortic blood flow into first and second renal arteries via respective first and second renal ostia having unique relative locations along the abdominal aorta wall. Moreover, such a system is generally provided with a local injection assembly and a flow isolation assembly.

According to one such aspect, the system includes a local injection assembly is provided in combination with a flow isolation assembly with a tubular wall having a longitudinal axis between a first end and a second end. The flow isolation assembly is adapted to be delivered to the location in a first condition with the tubular wall in a first configuration with a first diameter transverse to the longitudinal axis, and such that the first end is located upstream of the renal ostia and the second end is located downstream of the first end. The flow isolation assembly at the location is adjustable from the first condition to a second condition with the tubular wall in a second configuration as follows. The tubular wall in the second configuration has a second diameter that is greater than the first diameter and that is substantially constant between the first and second ends. According to this arrangement, a first region of abdominal aortic flow within an exterior flow path between the wall and the abdominal aortic wall is substantially isolated from a second region of abdominal aortic flow located within an interior flow path within the tubular wall, and the first and second regions of abdominal aortic blood flow are not substantially diverted by the tubular shaped wall. The local injection assembly is adapted to be fluidly coupled to a source of fluid agent located externally of the patient and to inject a volume of fluid agent from the source and into the first region between the abdominal aortic wall and the tubular wall in the second configuration at the location.

Another such aspect provides a local injection assembly in combination with a flow isolation assembly with a tubular wall having a longitudinal axis extending between a first end and a second end and also with a support member that is substantially ring-shaped and that is coupled to the tubular wall at one of the first and second ends. The flow isolation assembly is adapted to be delivered to the location in a first condition with the tubular wall in a first configuration and with the support member in a radially collapsed condition with a collapsed diameter transverse to the longitudinal axis, and further such that the first end is located upstream of the renal ostia and the second end is located downstream of the first end. At this location, the flow isolation assembly at the location is adjustable from the first condition to a second condition with the tubular wall in a second configuration and the support member in a radially extended condition with an extended diameter that is greater than the collapsed diameter. The support member in the radially extended condition supports the tubular wall at least in part in the second configuration with a tubular shape that is radially expanded relative to the first configuration with respect to the longitudinal axis. Accordingly, the assembly is adapted such that a first region of abdominal aortic flow within an exterior flow path between the tubular wall and the abdominal aortic wall is substantially isolated from a second region of abdominal aortic flow located within an interior flow path within the tubular wall. Of substantial benefit, the support member is constructed from a superelastic metallic wire with two opposite ends and a curved region between the two opposite ends that forms a substantially looped shape around a circumferential path. The wire has a memory shape with the two opposite ends at first and second memory positions relative to each other with respect to the circumferential path such that the curved region has a memory diameter that is less than the extended diameter. The wire in the flow isolation assembly is secured relative to the tubular member in a superelastically deformed condition with the two opposite ends at first and second displaced positions relative to each other such that the support member in the second configuration and with the extended diameter comprises a superelastically deformed condition for the wire. The local injection assembly is adapted to be fluidly coupled to a source of fluid agent located externally of the patient and to inject a volume of fluid agent from the source and into the first region with the flow isolation assembly in the second condition at the location.

Another aspect includes a local injection assembly in combination with a flow isolation assembly with a tubular wall having a longitudinal axis between a first end and a second end as follows. A retraction member is also provided in the system with a proximal end portion and a distal end portion that is coupled to the flow isolation assembly. The flow isolation assembly is adapted to be delivered to the location in a first condition with the tubular wall in a first configuration with a first diameter transverse to the longitudinal axis, and such that the first end is located upstream of the renal ostia and the second end is located downstream of the first end. The flow isolation assembly at the location is adjustable from the first condition to a second condition with the tubular wall in a second configuration. The tubular wall in the second configuration comprises a second diameter that is greater than the first diameter such that a first region of abdominal aortic flow within an exterior flow path between the tubular wall and the abdominal aortic wall is substantially isolated from a second region of abdominal aortic flow along an interior flow path within the tubular wall. Accordingly, the retraction member is adapted to adjust the tubular wall from the second configuration to a third configuration by proximal withdrawal of the proximal end portion of the retraction member externally of the patient. In this third configuration the tubular wall is partially retracted and has a third diameter that is less than the second diameter but greater than the first diameter. In addition, the local injection assembly is adapted to couple to a source of fluid agent located externally of the patient and to inject a volume of fluid agent from the source and into the first region with the flow isolation assembly in the second condition.

Another aspect also includes a local injection assembly with a flow isolation assembly with a tubular wall, an inflatable member, and an expandable member. The tubular wall has a first end, a second end, an outer surface, and an inner surface that defines a longitudinal passageway that extends along a longitudinal axis between the first and second ends. The inflatable member is located within the longitudinal passageway of the tubular wall and is adjustable between a deflated condition with a deflated diameter and an inflated condition with an inflated diameter that is greater than the deflated diameter. The tubular wall is adjustable, by inflating the inflatable member from the deflated condition to the inflated condition, from a first configuration with the longitudinal passageway having a first inner diameter transverse to the longitudinal axis to a second configuration with the longitudinal passageway having a second inner diameter that is greater than the first inner diameter. The inflatable member in the inflated condition does not completely occlude the longitudinal passageway of the tubular wall in the second configuration such that at least one flow passageway extends along the longitudinal passageway between the first and second ends. In addition, the expandable member is located on the outer surface of the tubular member and is adjustable between a radially collapsed condition relative to the outer surface and a radially expanded condition that is expanded from the outer surface of the tubular member relative to the radially collapsed condition. Also, the flow isolation assembly is adapted to be delivered to the location in a first condition that is characterized by the inflatable member in the deflated condition, the tubular wall in the first configuration, and the expandable member in the radially collapsed condition, and such that the first end is located upstream of the renal ostia and the second end is located downstream of the first end. The flow isolation assembly at the location is adjustable from the first condition to a second condition that is characterized by the inflatable member in the inflated condition, the tubular wall in the second configuration, the expandable member in the radially expanded condition. In the second condition at the location, the flow isolation assembly is adapted to substantially isolate a first region of abdominal aortic blood flow externally around the tubular member from a second region of abdominal aortic blood flow internally within the tubular member along the at least one flow passageway. The local injection assembly is adapted to couple to a source of fluid agent located externally of the patient and to inject a volume of fluid agent from the source and into the first region when the flow isolation assembly is in the second condition at the location.

Another aspect includes a delivery member with an elongate body with a proximal end portion and a distal end portion with a longitudinal axis and a circumference, and a bilateral local renal delivery assembly comprising a local injection assembly and a flow isolation assembly. The local injection assembly has a plurality of arms that are spaced circumferentially around the distal end portion. Each arm extends along the longitudinal axis between a proximal position and a distal position. The local injection assembly further includes a plurality of injection ports located along the plurality of arms, respectively, between the respective proximal and distal positions. The flow isolation assembly includes a wall assembly coupled to the plurality of arms. Accordingly, the bilateral local renal delivery assembly is adapted to be delivered with the distal end portion to the location in a first condition with the plurality of arms and wall assembly in a radially collapsed condition relative to the elongate body with the proximal end portion extending externally of the patient. The bilateral local renal delivery assembly is thus adjustable at the location from the first condition to a second condition wherein the plurality of arms and wall assembly are in a radially extended condition that is extended from the elongate body relative to the radially collapsed condition. In the second condition the arms and wall assembly form an expanded tubular wall that substantially isolates a first region of abdominal aortic blood flow along an exterior flow path between the tubular wall and the abdominal aortic wall from a second region of abdominal aortic blood flow along an interior flow path extending within the tubular wall between the proximal and distal positions, respectively. Also in the second condition at the location, the plurality of injection ports are fluidly coupled to the first region and are adapted to be fluidly coupled to a source of fluid agent located externally of the patient. The injection ports are adapted to inject a volume of fluid agent from the source and into the first region such that the injected volume flows substantially into the first and second renal arteries via the respective first and second renal ostia.

Another aspect provides a local injection assembly in combination with a flow isolation assembly with a wall that has a first portion and a second portion with a vent. The local injection assembly is adapted to be delivered to the location and to be fluidly coupled to a source of fluid agent located externally of the patient. In a first condition for the flow isolation assembly the wall is in a first configuration and is adapted to be delivered to the location. At the location, the flow isolation assembly is adjustable from the first condition to the second condition. In the second condition at the location, the first portion of the wall is adapted to isolate a first region from a second region of abdominal aortic blood flow at the location. The local injection assembly is adapted to cooperate with the flow isolation assembly so as to inject a volume of fluid agent from the source and into the first region at the location with the flow isolation assembly in the second condition at the location. Furthermore, the vent is adapted to allow the first region to communicate with the second region along the second portion.

Another aspect provides a local injection assembly in combination with a flow isolation assembly with a wall having a first end and a second end. The flow isolation assembly has a first condition with the wall in a first configuration and such that the flow isolation assembly is adapted to be delivered to the location with the first end located upstream of the renal ostia and with the second end located downstream of the first end. The flow isolation assembly at the location is adjustable from the first condition to a second condition wherein the wall is in a second configuration that is angled relative to a longitudinal axis of the abdominal aorta such that the first end is closer to a portion of the abdominal aorta wall than the second end and such that a first region of abdominal aortic blood flow between the wall and the portion is substantially isolated from a second region of abdominal aortic blood flow opposite the first region relative to the wall. The local injection assembly is adapted to couple to a source of fluid agent located externally of the patient and to inject a volume of fluid agent from the source and into the first region with the flow isolation assembly in the second condition at the location.

Another aspect is a local injection assembly in combination with a flow isolation assembly that is adjustable between a first condition and a second condition. The flow isolation assembly in the first condition is adapted to be delivered to the location. The flow isolation assembly at the location is adjustable from the first condition to the second condition. The flow isolation assembly in the second condition at the location is adapted to isolate fluid communication between a first region and a second region of abdominal aortic blood flow. The local injection assembly is adapted to couple to a source of fluid agent located externally of the patient and to inject a volume of fluid agent from the source and into the first region with the flow isolation assembly in the second condition at the location. Further to this aspect, the first region does not include a portion of an outer region of the abdominal aortic blood flow along the abdominal aortic wall.

Another aspect provides a local injection assembly with first and second injection ports in combination with a flow isolation assembly. The flow isolation assembly is adjustable between a first condition and a second condition as follows. In the first condition, the flow isolation assembly is adapted to be delivered to the location. The flow isolation assembly at the location is adjustable from the first condition to the second condition that is adapted to isolate fluid communication between a first region and a second region of abdominal aortic blood flow. The first and second injection ports are adapted to be delivered to first and second positions that are fluidly coupled with the first region when the flow isolation assembly is in the second condition at the location. The first and second injection ports at the first and second positions are adapted to be fluidly coupled to a source of fluid agent located externally of the patient and to simultaneously inject a volume of fluid agent from the source and into the first region such that the injected volume of fluid agent flows substantially into the first and second renal arteries, respectively, via the respective first and second renal ostia.

Another aspect provides a delivery member with an elongate body having a proximal end portion and a distal end portion and also a delivery lumen extending along a longitudinal axis between a proximal port along the proximal end portion and a distal port along the distal end portion, and also provides a local injection assembly that is adjustable between a first configuration and a second configuration The delivery lumen has a proximal portion with a first inner diameter along the proximal end portion, and has a distal portion with a second diameter that is greater than the first diameter along the distal end portion. The local injection assembly in the first configuration is located within the distal portion of the delivery lumen; wherein the distal end portion is adapted to be positioned with the local injection assembly in the first configuration at the location while the proximal end portion extends externally from the patient. The local injection assembly at the location is adapted to be fluidly coupled to a source of fluid agent located externally of the patient. The local injection assembly is adjustable at the location from the first configuration to the second configuration that is extended distally from the distal portion of the delivery lumen through the distal port and into the abdominal aorta at the location. Moreover, the local injection assembly in the second configuration at the location is adapted to inject a volume of fluid agent from the source and substantially into the first and second renal arteries.

Another aspect of the invention is a proximal coupler assembly for concurrent use with a bilateral local renal delivery device and percutaneous translumenal interventional device. This is of particular benefit where the bilateral local renal delivery device comprises an elongate body with a proximal end portion and a distal end portion and a local injection assembly located along the distal end portion. The system according to this aspect includes a housing with a distal end and a proximal end. The distal end includes a distal coupler that is adapted to be coupled to an introducer sheath that provides percutaneous translumenal access into a vasculature of a patient that leads to a location within an abdominal aorta associated with renal artery ostia. The proximal end comprises an adjustable hemostatic coupler that is adapted to simultaneously receive the bilateral local renal delivery device and the percutaneous translumenal device into the housing and is substantially aligned along a longitudinal axis with the distal end of the housing. Also included in this system are means for securing the proximal end portion of the bilateral local renal delivery device off-axis relative to the longitudinal axis so as to reduce interference between the percutaneous translumenal interventional device and the bilateral local renal delivery device when the percutaneous translumenal interventional device is manipulated within the hemostatic valve.

Another aspect of the invention is a method for treating a renal system in a patient from a location within the abdominal aorta associated with abdominal aortic blood flow into each of first and second renal arteries via first and second renal ostia, respectively, at unique respective locations along the abdominal aorta wall.

One such method includes positioning a local injection assembly at the location; fluidly coupling to the local injection assembly at the location to a source of fluid agent externally of the patient; and injecting a volume of fluid agent from the source and into the abdominal aorta at the location in a manner such that the injected fluid flows principally into the first and second renal arteries via the first and second renal ostia, respectively, and without substantially occluding or isolating a substantial portion of an outer region of aortic blood flow along a circumference of the abdominal aorta wall and across the location.

Another method aspect includes delivering a flow isolation assembly with a tubular wall having a longitudinal axis between a first end and a second end to the location in a first condition with the tubular wall in a first configuration with a first diameter transverse to the longitudinal axis, and such that the first end is located upstream of the renal ostia and the second end is located downstream of the first end. Also included is adjusting the flow isolation assembly at the location from the first condition to a second condition with the tubular wall in a second configuration that comprises a second diameter that is greater than the first diameter and that is substantially constant between the first and second ends such that a first region of abdominal aortic flow within an exterior flow path between the wall and the abdominal aortic wall is substantially isolated from a second region of abdominal aortic flow located within an interior flow path within the tubular shaped wall, and further such that the first and second regions of abdominal aortic blood flow are not substantially diverted by the tubular shaped wall. Further includes is fluidly coupling a local injection assembly to a source of fluid agent located externally of the patient. Also included is injecting a volume of fluid agent from the source and into the first region between the abdominal aortic wall and the tubular wall in the second configuration at the location.

Another method aspect includes delivering a flow isolation assembly with a tubular wall to the location in a first condition with the tubular wall in a first configuration and with a support member in a radially collapsed condition with a collapsed diameter transverse to a longitudinal axis of the tubular wall, and such that a first end of the tubular wall is located upstream of the renal ostia and a second end of the tubular wall is located downstream of the first end. Another step of this aspect includes adjusting the flow isolation assembly at the location from the first condition to a second condition with the tubular wall in a second configuration and the support member in a radially extended condition with an extended diameter that is greater than the collapsed diameter. Still a further step includes: supporting the tubular wall in the second configuration with the support member in the radially extended condition such that the tubular wall has a tubular shape that is radially expanded relative to the first configuration with respect to the longitudinal axis, and such that a first region of abdominal aortic flow within an exterior flow path between the tubular wall and the abdominal aortic wall is substantially isolated from a second region of abdominal aortic flow located within an interior flow path within the tubular wall. This method is of particular benefit wherein the support member includes a superelastic metallic wire with two opposite ends and a curved region between the two opposite ends that forms a substantially looped shape around a circumferential path, and the support member in the second configuration and with the extended diameter includes a superelastically deformed condition for the wire. Another step includes fluidly coupling the local injection assembly to a source of fluid agent located externally of the patient. A further step is: injecting a volume of fluid agent with the local injection assembly from the source and into the first region with the flow isolation assembly in the second condition at the location.

Another aspect of the invention includes a method for treating a renal system in a patient from a location within the abdominal aorta associated with abdominal aortic blood flow into first and second renal arteries via respective first and second renal ostia having unique relative locations along the abdominal aorta wall. This further method includes: providing a local injection assembly and a flow isolation assembly with a tubular wall having a longitudinal axis between a first end and a second end. Also included is using a retraction member with a proximal end portion and a distal end portion that is coupled to the flow isolation assembly to control the flow isolation assembly. This method further includes delivering a flow isolation assembly to the location in a first condition with a tubular wall in a first configuration with a first diameter transverse to a longitudinal axis within the tubular wall, and such that a first end of the tubular wall is located upstream of the renal ostia and a second end of the tubular wall is located downstream of the first end. Also included is adjusting the flow isolation assembly at the location from the first condition to a second condition with the tubular wall in a second configuration that comprises a second diameter that is greater than the first diameter such that a first region of abdominal aortic flow within an exterior flow path between the tubular wall and the abdominal aortic wall is substantially isolated from a second region of abdominal aortic flow along an interior flow path within the tubular shaped wall. A further step is adjusting the tubular wall from the second configuration to a third configuration by proximal withdrawal of a proximal end portion of a retraction member externally of the patient, wherein a distal end portion of the retraction member is coupled to the tubular wall, and such that in the third configuration the tubular wall is partially retracted and has a third diameter that is less than the second diameter but greater than the first diameter. Still further is coupling a local injection assembly to a source of fluid agent located externally of the patient, and injecting a volume of fluid agent with the local injection assembly from the source and into the first region with the flow isolation assembly in the second condition.

Another method aspect includes as a step: delivering a flow isolation assembly to the location in a first condition that is characterized by an inflatable member within a longitudinal passageway of a tubular wall in a deflated condition with a deflated diameter, the tubular wall in a first configuration, and an expandable member on an outer surface of the tubular wall in a radially collapsed condition, and such that a first end of the tubular wall is located upstream of the renal ostia and a second end of the tubular wall is located downstream of the first end. Also included is the following step: adjusting the flow isolation assembly at the location from the first condition to a second condition by inflating the inflatable member to an inflated condition with an inflated diameter that is greater than the deflated diameter and that expands the tubular wall such that the longitudinal passageway has a second inner diameter that is greater than the first inner diameter, and also by expanding the expandable member to a radially expanded condition that is expanded from the outer surface of the tubular member relative to the radially collapsed condition, and further such that the inflatable member in the inflated condition does not completely occlude the longitudinal passageway of the tubular wall in the second configuration so as to provide at least one flow passageway extending along the longitudinal passageway between the first and second ends. Still a further step includes substantially isolating a first region of abdominal aortic blood flow externally around the tubular member from a second region of abdominal aortic blood flow internally within the tubular member along the at least one flow passageway with the flow isolation assembly in the second condition at the location. Another step is: coupling a local injection assembly to a source of fluid agent located externally of the patient; and injecting a volume of fluid agent with the local injection assembly from the source and into the first region when the flow isolation assembly is in the second condition at the location.

Another method aspect includes providing a delivery member with an elongate body with a proximal end portion and a distal end portion with a longitudinal axis and a circumference; providing a bilateral local renal delivery assembly with a local injection assembly and a flow isolation assembly, wherein the local injection assembly comprises a plurality of arms that are spaced circumferentially around the distal end portion, wherein each arm extends along the longitudinal axis between a proximal position and a distal position, wherein the local injection assembly further comprises a plurality of injection ports located along the plurality of arms, respectively, between the respective proximal and distal positions, and wherein the flow isolation assembly comprises a wall assembly coupled to the plurality of arms; delivering the bilateral local renal delivery assembly with the distal end portion of the elongate body of the delivery member to the location in a first condition with a plurality of arms and wall assembly in a radially collapsed condition relative to the elongate body while a proximal end portion of the elongate body extends externally of the patient; adjusting the bilateral local renal delivery assembly at the location from the first condition to a second condition wherein the plurality of arms and wall assembly are in a radially extended condition that is extended from the elongate body relative to the radially collapsed condition; forming an expanded tubular wall with the arms and wall assembly in the second condition; substantially isolating a first region of abdominal aortic blood flow along an exterior flow path between the tubular wall and the abdominal aortic wall, and a second region of abdominal aortic blood flow along an interior flow path extending within the tubular wall between proximal and distal ports adjacent to and located between the proximal and distal positions, respectively, with the expanded tubular wall at the location. According to another step in the second condition at the location, fluidly coupling the plurality of injection ports to the first region and also to a source of fluid agent located externally of the patient. Further included is injecting a volume of the fluid agent with the injection ports from the source and into the first region such that the injected volume flows substantially into the first and second renal arteries via the respective first and second renal ostia.

Another method aspect includes delivering a flow isolation assembly in a first condition with a wall in a first configuration to the location; fluidly coupling the local injection assembly at the location to a source of fluid agent located externally of the patient; adjusting the flow isolation assembly at the location from the first condition to a second condition wherein a first portion of the wall is adapted to isolate a first region from a second region of abdominal aortic blood flow at the location; injecting a volume of fluid agent with a local injection assembly from the source and into the first region at the location with the flow isolation assembly in the second condition at the location; and allowing the first region to communicate with the second region through a vent located along a second portion of the wall.

Another method aspect includes delivering a flow isolation assembly in a first condition to the location with a wall in a first configuration and a first end of the wall located upstream of the renal ostia and a second end of the wall located downstream of the first end; adjusting the flow isolation assembly at the location from the first condition to a second condition with the wall in a second configuration that is angled relative to a longitudinal axis of the abdominal aorta such that the upstream end is closer to a portion of the abdominal aorta wall than the downstream end and such that a first region of abdominal aortic blood flow between the wall and the portion is substantially isolated from a second region of abdominal aortic blood flow opposite the first region relative to the wall; coupling a local injection assembly to a source of fluid agent located externally of the patient; and injecting a volume of fluid agent with the local injection assembly from the source and into the first region while the flow isolation assembly is in the second condition at the location.

Another method aspect includes delivering a flow isolation assembly in a first condition to the location; adjusting the flow isolation assembly at the location from the first condition to a second condition wherein the flow isolation assembly is adapted to isolate fluid communication between a first region and a second region of abdominal aortic blood flow and wherein the first region does not include a portion of an outer region of the abdominal aortic blood flow along the abdominal aortic wall; coupling the local injection assembly to a source of fluid agent located externally of the patient; and injecting a volume of fluid agent from the source and into the first region while the flow isolation assembly is in the second condition at the location.

Another method aspect includes delivering a flow isolation assembly in a first condition to the location; adjusting the flow isolation assembly at the location from the first condition to a second condition that is adapted to isolate fluid communication between a first region and a second region of abdominal aortic blood flow; delivering first and second injection ports of a local injection assembly to first and second positions that are fluidly coupled with the first region when the flow isolation assembly is in the second condition at the location; fluidly coupling the first and second injection ports at the first and second positions to a source of fluid agent located externally of the patient; and simultaneously injecting a volume of fluid agent from the source and into the first region such that the injected volume of fluid agent flows substantially into the first and second renal arteries, respectively, via the respective first and second renal ostia.

Another method aspect includes providing a delivery member with an elongate body having a proximal end portion and a distal end portion and also a delivery lumen extending along a longitudinal axis between a proximal port along the proximal end portion and a distal port along the distal end portion; providing a local injection assembly that is adjustable between a first configuration and a second configuration. The delivery lumen has a proximal portion with a first inner diameter along the proximal end portion, and has a distal portion with a second diameter that is greater than the first diameter along the distal end portion. Another step is positioning a local injection assembly in the first configuration within the distal portion of the delivery lumen. Still another is delivering the distal end portion with the local injection assembly in the first configuration at the location while the proximal end portion extends externally from the patient. A further step includes fluidly coupling the local injection assembly at the location to a source of fluid agent located externally of the patient; adjusting the local injection assembly at the location from the first configuration to a second configuration that is extended distally from the distal portion of the delivery lumen through the distal port and into the abdominal aorta at the location; and injecting a volume of fluid agent from the source and substantially into the first and second renal arteries with the local injection assembly in the second configuration at the location.

Further aspects of the invention will be brought out in the following portions of the specification, wherein the detailed description is for the purpose of fully disclosing preferred embodiments of the invention without placing limitations thereon.

### BRIEF DESCRIPTION OF THE DRAWINGS

The invention will be more fully understood by reference to the following drawings which are for illustrative purposes only:

FIG. 1 is a schematic illustration of the natural blood flow patterns of the aorta and renal arteries.

FIG. 2 illustrates an aortic flow diverter with a single hoop and double walled skirt.

FIG. 3 is a schematic illustration of the aortic flow diverter in FIG. 2 inserted in the aorta above the renal arteries.

FIG. 4 is an illustration of another aortic flow diverter with two hoops.

FIG. 5 is a partial side section view of the aortic flow diverter in FIG. 4 inserted in the aorta above the renal arteries.

FIG. 6A illustrates a side view of an aortic flow diverter with a skirt with a partial conical shape.

FIG. 6B illustrates a dorsal view of the aortic flow diverter shown in FIG. 6A.

FIG. 6C illustrates the aortic flow diverter shown in FIG. 6A inserted in the aorta near the renal arteries.

FIG. 7 illustrates an aortic flow diverter with the distal hoop larger than the proximal hoop and holes to direct aortic blood flow.

FIG. 8 shows the aortic flow diverter of FIG. 7 positioned in the aorta above the renal arteries.

FIG. 9 illustrates a metal frame of a scalloped shape aortic flow diverter.

FIG. 10 illustrates the fabric covering the frame shown in FIG. 9.

FIG. 11 illustrates the scallop shaped flow diverter shown in FIG. 10 in a bifurcated configuration and positioned in the aorta.

FIG. 12 is a top cross section view of the scallop shape flow diverter shown in FIG. 1 positioned in the aorta.

FIG. 13 illustrates the catheter cross section of another mode of deploying a scallop shaped flow diverter as shown in FIG. 11.

FIG. 14. illustrates the retraction of the scallop shaped flow diverter as shown in FIG. 11 into a sheath.

FIG. 15 Illustrates a catheter with an enlarged distal tip adapted to deliver an aortic flow diverter.

FIG. 16 illustrates schematically the positioning of the aortic flow diverter shown in FIG. 15 in the aorta system.

FIG. 17 illustrates schematically the aortic flow diverter shown in FIG. 16 deployed near the renal arteries in an expanded state.

FIG. 18 illustrates an aortic flow diverter with a pull wire in a partially collapsed state.

FIG. 19 illustrates the aortic flow diverter shown in FIG. 18 deployed into an expanded state by relaxing the pull wire.

FIG. 20 illustrates a variation of the aortic flow diverter FIG. 18 that adapts to a collapsed shape using a pulley assembly with a pull wire.

FIG. 21 illustrates the aortic flow diverter in FIG. 20 deployed into an expanded state by relaxing the pull wire.

FIG. 22 illustrates positioning of the aortic flow diverter shown in FIG. 21 with a proximal hub assembly and introducer sheath.

FIG. 23 shows schematically an aortic flow diverter configured as a collar around a guide catheter.

FIG. 24 shows another embodiment of the aortic flow diverter in FIG. 23 where an expandable tubular member is placed on a fluid delivery lumen.

FIG. 25 illustrates schematically a fluid agent delivery system where a guide catheter is a dual lumen extrusion.

FIG. 26 illustrates schematically another fluid delivery system where the guide catheter has three lumens and an inflatable member.

FIG. 27 illustrates schematically another fluid delivery catheter where an aortic flow diverter assembly is attached to the catheter at a position downstream of a fluid delivery port.

FIG. 28. is an illustration of an expandable aortic flow diverter.

FIG. 29 illustrates the expandable aortic flow diverter shown in FIG. 28 in a collapsed state.

FIG. 30 is a schematic illustration of the expandable aortic flow diverter shown in FIG. 28 positioned in an aorta.

FIG. 31 illustrates an expandable aortic flow diverter adapting to a small aorta.

FIG. 32 illustrates an expandable aortic flow diverter adapting to a large aorta.

FIG. 33A illustrates a hoop for an aortic flow diverter, formed of a superelastic alloy, in its expanded condition and in its relaxed, zero strain state.

FIG. 33B illustrates a hoop, formed of a superelastic alloy, for an aortic flow diverter in its relaxed, zero strain state, where it is configured smaller than its expanded condition but larger than its collapsed condition.

FIG. 34A illustrates a typical stress strain graph for compressing the hoop configuration shown in FIG. 33A.

FIG. 34B illustrates a typical stress strain graph for compressing the hoop configuration shown in FIG. 33B.

FIG. 35 illustrates a tool for forming an aortic flow diverter hoop shown in FIG. 33A.

FIG. 36 illustrates a tool for forming an aortic flow diverter hoop shown in FIG. 33B.

FIG. 37 illustrates a first step in forming lumens for an aortic flow diverter starting with a tube of ePTFE material, extruded with multiple lumens.

FIG. 38 is a cross section of the tube shown in FIG. 37 illustrating the position of lumens and the position for making an axial cut line.

FIG. 39 illustrates the sheet formed from the tube shown in FIG. 37 after the axial cut.

FIG. 40 illustrates a flow diverter clip assembly.

FIG. 41 illustrates a variation of the flow diverter clip assembly shown in FIG. 40.

FIG. 42 illustrates another beneficial embodiment of the flow diverter clip assembly shown in FIG. 40.

FIG. 43 illustrates the left or right placement of a flow diverter clip assembly as shown in FIG. 40 on a patient.

FIG. 44 illustrates the position of a left flow diverter clip assembly shown in FIG. 43 with an aortic flow diverter and a catheter inserted in the aorta.

FIG. 45 is an aortic flow diverter where the elongated expandable member is formed from an elastomer-encased braided tube.

FIG. 46 illustrates the aortic flow diverter shown in FIG. 45 with the elongated expandable member changed to a shorter, larger diameter state.

FIG. 47 illustrates the aortic flow diverter shown in FIG. 45 located in the aorta with the expandable tubular member inflated and positioned downstream of the renal arteries.

FIG. 48 is a transverse cross sectional view of the aortic flow diverter shown in FIG. 47 taken along line 48-48.

FIG. 49 is a transverse cross sectional view of the aortic flow diverter shown in FIG. 47 taken along line 49-49.

FIG. 50 is a transverse cross sectional view of the aortic flow diverter shown in FIG. 47 taken along line 50-50.

FIG. 51 is an enlarged view, partially in phantom, of an aortic flow diverter having an expandable tubular sheath member over a collapsible frame and an inflatable member.

FIG. 52 is an enlarged view, partially in phantom, of an aortic flow diverter having a radially expandable sheath member with a radially enlarged section.

FIG. 53A is a transverse cross sectional view of another embodiment having an expandable tubular member with a small profile wrapped configuration.

FIG. 53B is a transverse cross sectional view of the tubular member shown In FIG. 53A, illustrating the tubular member in the expanded unwrapped configuration.

FIG. 54A is a transverse cross sectional view of another embodiment having an expandable tubular member with a small profile wound configuration.

FIG. 54B is a transverse cross sectional view of the tubular member shown in FIG. 54A, illustrating the tubular member in the expanded unwound configuration.

FIG. 55A illustrates an expandable tubular member wound like a rolled awning.

FIG. 55B illustrates the tubular member of FIG. 55A in the expanded unwound configuration.

FIG. 56 illustrates a transverse cross sectional view in which the tubular member comprises a plurality of inflatable balloons within an outer sheath in a non-inflated low profile configuration.

FIG. 57 illustrates the tubular member shown in FIG. 56 in an expanded state.

FIG. 58 illustrates another embodiment of an aortic flow diverter in section view with an inner inflatable member formed in a helical shape.

FIG. 59 illustrates an aortic flow diverter with the tubular member supported on a frame.

FIG. 60 illustrates an embodiment of an aortic flow diverter with an inner inflatable member encased in a sheath and an outer inflatable member.

FIG. 61 illustrates another variation of the aortic flow diverter shown in FIG. 60 where four inner inflatable tubular members present a four lobed, clover shape, cross section.

FIG. 62 illustrates a proximal coupler system for positioning aortic fluid delivery systems adjunctively with other medical devices.

FIG. 63 illustrates a section view of the proximal coupler system as shown in FIG. 62.

FIG. 64A illustrates a proximal coupler system as shown in FIG. 62 coupled to a local fluid delivery system.

FIG. 64B illustrates a proximal coupler system as shown in FIG. 64A with a fluid delivery system advanced into an introducer sheath.

FIG. 65 illustrates a proximal coupler system as shown in FIG. 54 through 56B with an aortic flow diverter positioned near the renal arteries and a catheter deployed adjunctively in the aorta.

FIG. 66 illustrates a proximal coupler assembly and fluid delivery assembly as shown in FIG. 65 as components of a renal therapy system including an introducer sheath system and a vessel dilator.

### DETAILED DESCRIPTION OF THE INVENTION

The description herein provided relates to medical methods to divert blood flow from a major blood vessel into one or more branch vessels.

For the purpose of providing a clear understanding, the term proximal should be understood to mean locations on a system or device relatively closer to the operator during use, and the term distal should be understood to mean locations relatively further away from the operator during use of a system or device.

These present embodiments below therefore generally relate to treatment at the renal arteries, generally from the aorta. However, it is contemplated that these systems and methods may be suitably modified for use in other anatomical regions and for other medical conditions without departing from the broad scope of various of the aspects illustrated by the embodiments.

As will be appreciated by reference to the detailed description below and in further respect to the Figures, the present invention is principally related to selective aortic flow diverter systems and methods, which are thus related to subject matter disclosed in the following prior filed, co-pending U.S. patent applications that are commonly owned with the present application: Ser. No. 09/229,390 to Keren et al., filed Jan. 11, 1999; Ser. No. 09/562,493 to Keren et al., filed May 1, 2000; and Ser. No. 09/724,691 to Kesten et al., filed Nov. 28, 2000. The disclosures of these prior patent applications are herein incorporated in their entirety by reference thereto.

The invention is also related to certain subject matter disclosed in other Published International Patent Applications as follows: WO 00/41612 to Libra Medical Systems, published Jul. 20, 2000; and WO 01/83016 to Libra Medical Systems, published Nov. 8, 2001. The disclosures of these Published International Patent Applications are also herein incorporated in their entirety by reference thereto.

In general, the disclosed material delivery systems will include a flow diverter assembly, a proximal coupler assembly and one or more elongated bodies, such as wires, tubes or catheters. These elongated bodies may contain one or more lumens and generally consist of a proximal region, a mid-distal region, and a distal tip region. The distal tip region will typically have means for diverting blood flow from a major vessel, such as an aorta, to a branch vessel, such as a renal artery. The distal tip region may also have a device for delivering a material such as a fluid agent. Radiopaque markers or other devices may be coupled to the specific regions of the elongated body to assist introduction and positioning.

The flow diverter and/or the material delivery system is intended to be placed into position by a physician, typically either an interventionalist (cardiologist or radiologist) or an intensivist, a physician who specializes in the treatment of intensive-care patients. The physician will gain access to a femoral artery in the patient's groin, typically using a Seldinger technique of percutaneous vessel access or other conventional method.

In addition, various of the embodiments are illustrated as catheter implementations, and are further illustrated during in-vivo use. Other techniques for positioning the required flow diverter assemblies described may be used where appropriate, such as transthoracic or surgical placement that either use or don't use percutaneous translumenal catheter techniques. In addition, reference to the illustrative catheter embodiments thus portray specific proximal-distal relationships between the inter-cooperating components of a flow diverter in relation to blood flow and their relative orientations on a delivery catheter platform. For example, some embodiments illustrate or are otherwise described by reference to retrograde femoral approach to renal delivery, such that the distal end of the catheter including the aortic flow diverter is located upstream form the proximal end of the catheter. Other embodiments may show an opposite relative positioning, such as via an antegrade access to the site of renal arteries, e.g. from a brachial or radial arterial access procedure. However, it is to be further understood that such embodiments, though shown or described in relation to one such mode, may be appropriately modified by one of ordinary skill for use in the other orientation approach without departing from the intended scope.

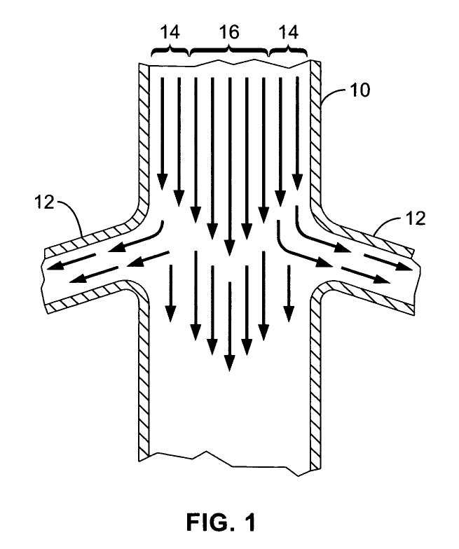

FIG. 1 shows a schematic cross-section of the abdominal aorta 10 taken in the immediate vicinity of the renal arteries 12. FIG. 1 shows the natural flow patterns through the abdominal aorta 10 and the natural flow patterns from the abdominal aorta 10 into the renal arteries 12. As shown, the flow down the abdominal aorta 10 maintains a laminar flow pattern. The flow stream along the wall of the abdominal aorta 10, as indicated by flow lines 14 contains a natural laminar flow stream into the branching arteries, e.g., the renal arteries 12. Moreover, the flow stream near the middle of the abdominal aorta 10, as indicated by flow pattern 16 continues down the abdominal aorta 10 and does not feed into any of the side branches, e.g., the renal arteries 12. As such, a drug solution infusion down the middle of the abdominal aorta flow stream can be ineffective in obtaining isolated drug flow into the renal arteries 12.

In general, the flow stream 16 is of a higher velocity than flow stream 14 along the wall of aorta 10. It is to be understood that near the boundaries of flow stream 14 with flow stream 16, there can be flow streams into the branching renal arteries 12 as well as down the abdominal aorta 10.

Further, the ostia of renal arteries 12 are positioned to receive substantial blood flow from the blood flow near the posterior wall of aorta 10 as well as the side walls. In other words, blood flow 14 is greater than blood flow 16 when along the posterior wall of aorta 10 relative to blood flow in the center of aorta 10 as shown in FIG. 1. Thus, drug infusion above renal arteries 12, and along the posterior wall of aorta 10, will be effective in reaching renal arteries 12.

Accordingly, in order to maximize the flow of a drug solution into the renal arteries using the natural flow patterns shown in FIG. 1, it is beneficial to provide a device, as described in detail below, that is adapted to selectively infuse a drug solution along the side wall or posterior wall of the abdominal aorta 10 instead of within the middle of the abdominal aorta 10 or along the anterior wall.

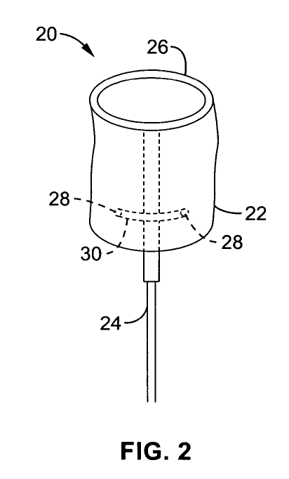

FIG. 2 illustrates a beneficial embodiment of an aortic flow diverter 20 with a circular skirt 22 of sheet material, such as ePTFE, attached to catheter lumen 24 and supported by metal wire hoop 26. Two infusion ports 28 are placed in the outside of skirt 22 approximately 90 degrees to about 180 degrees apart and are fluidly connected to catheter lumen 24 through fluid channels 30. The single hoop 26 allows for sizing to an aorta 10 to maintain the infusion ports 28 along the inner wall of aorta 10. The particular embodiment shown allows advancement of a interventional catheter (not shown) through the open center of device 20 and does not alter blood flow. The embodiment shown in FIG. 2 reduces the presence of stagnant blood thereby minimizing the occurrence of blood clotting on aortic flow diverter 20. It is to be appreciated that wire hoop 26 can be adjusted between a collapsed condition, such as radially constrained in a sheath, and an expanded condition as shown in FIG. 1. In one exemplary embodiment, Aortic flow diverter 20 is about 1.5 cm in total length.

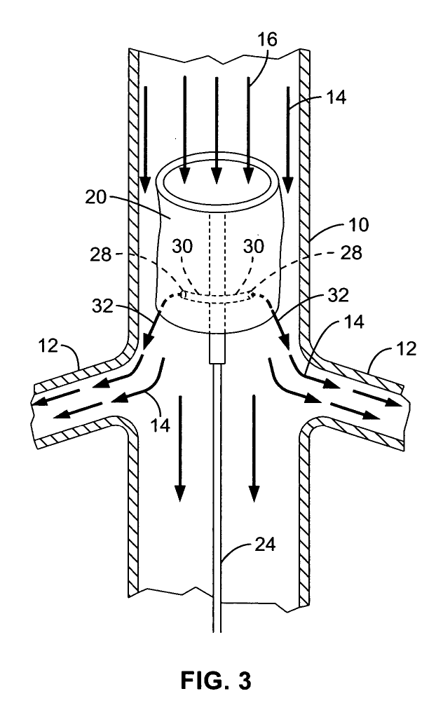

FIG. 3 is a schematic dorsal view of the aortic flow diverter 20 shown in FIG. 2 placed upstream of renal arteries 12 in aorta 10. Fluid agent 32 flows through catheter lumen 24, through fluid channels 30 and out of infusion ports 28. Fluid agent 32 is carried by outer blood flow 14 into renal arteries 12. In one embodiment, catheter lumen 24 has an offset that is a slight S shape (not shown) and positions aortic flow diverter 20 off the aorta wall 10.

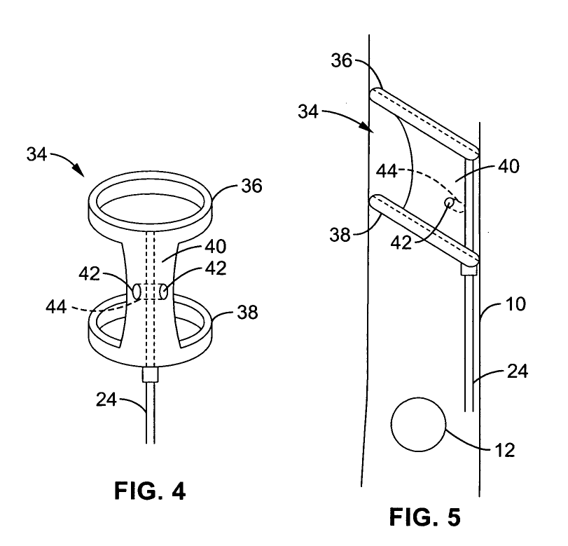

FIG. 4 shows another embodiment of an aortic flow diverter 34 comprising a distal metal wire hoop 36 and a proximal metal wire hoop 38 connected to catheter 24 to form parallel circular openings perpendicular to catheter 24. In a beneficial embodiment, distal hoop 36 and proximal hoop 38 are about 2 centimeters apart. A partial skirt 40, of material such as ePTFE, is attached and supported by distal hoop 36, proximal hoop 38, and extends along the spine or dorsal side of catheter lumen 24. Approximately 50 percent to about 75 percent of partial skirt 40 is cut away in an area bounded by distal hoop 36, proximal hoop 38 and the hoop circumferences opposite catheter lumen 24 so that partial skirt 40 assumes an hourglass shape between distal hoop 36 and proximal hoop 38 and symmetrical about catheter lumen 24. Infusion ports 42 are fluidly connected to catheter lumen 24 through fluid channels 44 and placed midway between distal hoop 34 and proximal hoop 36 on the edges of partial skirt 38. It is to be appreciated that distal hoop 36 and proximal hoop 38 can be adjusted between a collapsed condition, such as radially constrained in a sheath, and an expanded condition as shown. In one embodiment, catheter lumen 24 has an offset that is a slight S shape (not shown) and positions the aortic flow diverter off the aorta wall.

FIG. 5 is a schematic illustration of the aortic flow diverter shown in FIG. 4 inserted in aorta 10 above renal arteries 12. Wire hoops 36 and 38 contact the inner wall of aorta 10 and flex at the joint with catheter lumen 24. This presses the dorsal side of partial skirt 40 against the inner wall of aorta 10 and places infusion ports 42 near the dorsal aorta wall and above renal arteries 12. This particular embodiment allows one device size to be used on many different sized aorta. The reduced material in the blood stream of partial skirt 40 beneficially reduces the occurrence of stagnant blood and blood clotting.

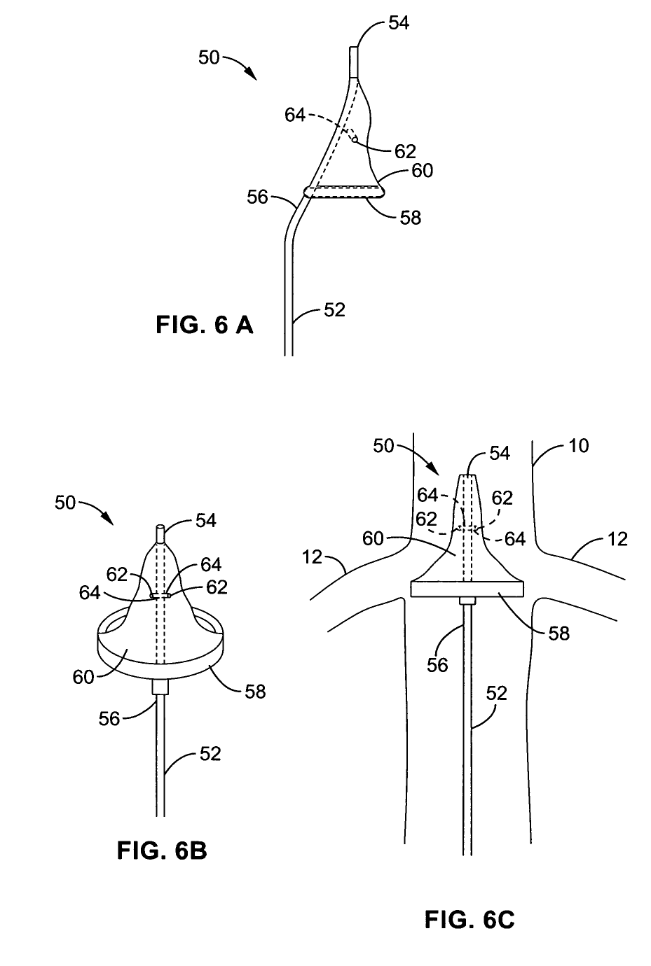

FIG. 6A through FIG. 6C illustrate another embodiment of an aortic flow diverter 50 where FIG. 6A is a side view and FIG. 6B is a dorsal view. Catheter lumen 52 has a distal end 54 and a mid proximal position 56. A circular wire hoop 58, made of a flexible memory shape material, is coupled in an approximately perpendicular orientation to catheter lumen 52 at mid proximal position 56. A partial conical skirt 60 extends from the distal end 54 of catheter lumen 52 to proximal wire hoop 58. Conical skirt 60, made from a sheet material such as ePTFE, is cut away lengthwise and on the opposite side of catheter lumen 52. Covering only one-half the conical shape reduces stagnant blood and the chance of blood clot formation. Infusion ports 62 are in fluid communication with catheter lumen 52 through fluid channels 64 in conical skirt 60. In the embodiment shown here, catheter lumen 52 has an offset adaptation between mid proximal position 56 and distal end 54 to optimally position aortic flow diverter 50 in the aorta 10.

FIG. 6C shows the aortic flow diverter 50 shown in FIG. 6A and FIG. 6B positioned near renal arteries 12 in aorta 10. It is to be appreciated that circular wire hoop 58 can be adjusted between a collapsed condition, such as radially constrained in a sheath, and an expanded condition as shown.

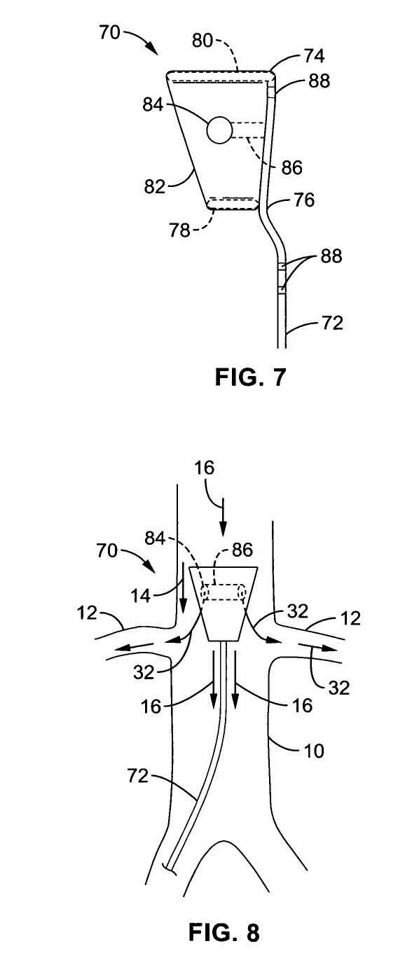

FIG. 7 is another embodiment of an aortic flow diverter 70. Catheter 72, shown in partial section view, has a distal end 74 and a mid proximal position 76. A proximal wire hoop 78, made of a flexible memory shape material, is coupled in an approximately perpendicular orientation to catheter lumen 72 at mid proximal position 76. A distal wire hoop 80 is coupled at distal end 74 and is larger in diameter than proximal wire hoop 78. Skirt 82, made of a fabric or sheet material such as ePTFE, is attached to hoop 72, hoop 74 and catheter lumen 24 forming a funnel. Holes 84 are placed symmetrically on opposite sides of catheter 72 and placed midway between distal hoop 80 and proximal hoop 78 in skirt 82. Fluid agent is delivered from catheter 72 through infusion channels 86 and exits infusion channels 86 at or near holes in fabric 84 (shown in FIG. 8). In one beneficial embodiment, radiopaque marker bands 88 are coupled to catheter 72 at distal end 74 and mid proximal position 76 to aid in positioning.

FIG. 8 is aortic flow diverter 70 shown in FIG. 7 deployed in aorta 10 above renal arteries 12. Blood flow 14 flows alongside aortic flow diverter 70 and into the renal arteries. Blood flow 16 flows through the center of aortic flow diverter 70 and past renal arteries 12. Some of blood flow 16 flows along the wall inside of aortic flow diverter 70 with some flowing out through holes 84, delivering fluid agent 32 from infusion channel 86 to blood flow 14 and flowing down the wall of aorta 10 and into renal arteriesl 2.

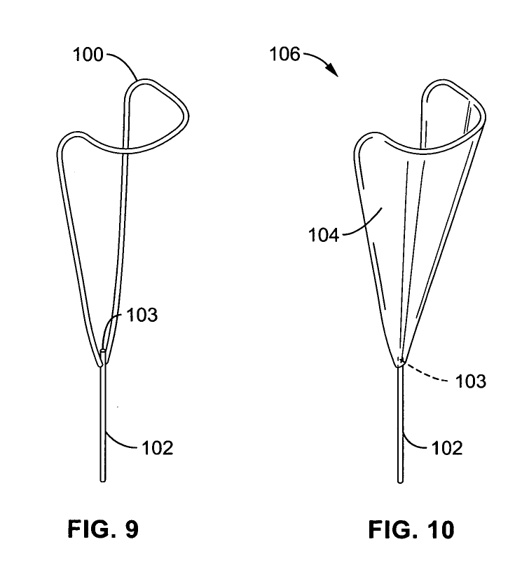

FIG. 9 through FIG. 12 illustrates a double scallop shaped flow diverter.

In FIG. 9, a metal frame 100 is shaped in a scallop shape by making an arc loop and bending it 90 degrees. The ends of frame 100 are formed into a “V” shape. Agent delivery tube 102 with agent delivery port 103 is coupled to frame 100 at the wire ends.

FIG. 10 illustrates a fabric covering 104 fastened over frame 100 to form a semi conical scallop assembly 106. Agent delivery port 103, at the distal end of agent delivery tube 102 is on the concave side of fabric 104. Because fabric 104 is supported by frame 100, it maintains a predictable shape during use.

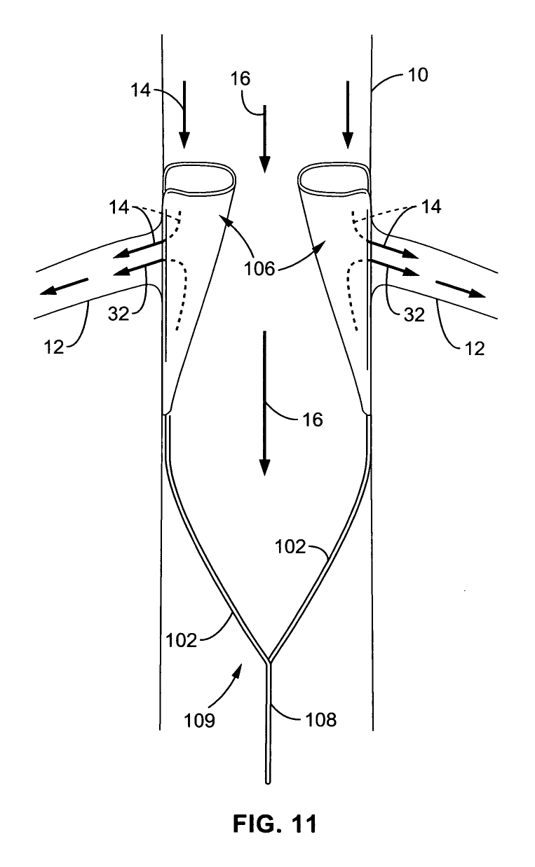

In FIG. 11, two scallop assemblies 106 as shown in FIG. 10, with concave surfaces facing outward, are connected by a center tube 108 in fluid communication with agent delivery tubes 102 and agent delivery ports 103 (not shown for clarity) to form a bifurcated scallop assembly 109. The concave face of each scallop assembly 106 is sealed against the walls of aorta 10 at renal arteries 12. In one beneficial mode, an outward spring force in agent delivery tubes 102 keeps the scallop assemblies 106 in place against the aorta wall. Because the spring force can have a wide range, one bifurcated scallop assembly 109 can be used on different sized aorta. In another mode, radio opaque markers (not shown) at strategic locations such as on the top loop of wire 100 and at the union of agent delivery tubes 102, aid in positioning of the bifurcated scallop assembly 109. In a further mode, each scallop assembly 106 is introduced independently on agent delivery tube 102 from a proximal coupler assembly (not shown). Blood flow 14 flowing near the wall of aorta 10 is diverted by the arc end of scallop assembly 106 to the concave face of scallop assembly 106 where it mixes with fluid agent 32 flowing from agent delivery tubes 102 and perfuses into the renal arteries 12. Blood flow 16 near the center of aorta 10 flows past scallop assembly 106.

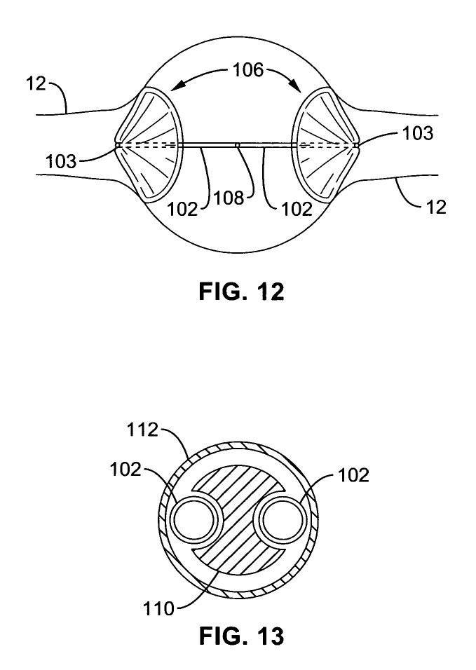

FIG. 12 is a cross section of FIG. 11 showing the placement of scallop assemblies 106 against the wall of aorta 10 upstream of renal arteries 12 and the position of agent delivery port 103.

FIG. 13 illustrates a further beneficial mode of the scallop assemblies 106 shown in FIG. 11 wherein a supporting member 110, shown in cross section, is positioned inside sheath 112 and engages a section of agent delivery tubes 102 below the “V” of wires 100 for both scallop assemblies 106. Supporting member 110 is connected to controls in a proximal coupler assembly (not shown) and aids in rotating scallop assemblies 106 during insertion and positioning from sheath 112. Supporting member 110 is configured to be removed proximally from sheath 112 once scallop assemblies 106 (shown in FIG. 12) are positioned at the renal arteries 12.

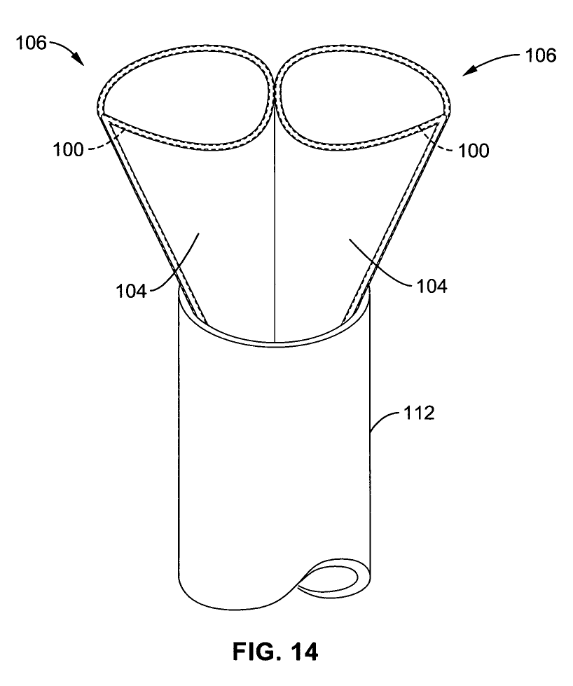

FIG. 14 illustrates the withdrawal of scallop assemblies 106 into sheath 112. Sheath 112 forces the “V” legs of wire frame 100 together so that scallop assemblies 106 form a cone with the opening pointing upstream. This cone configuration helps capture thrombus that has formed during the medical procedure and is flowing in the aorta.

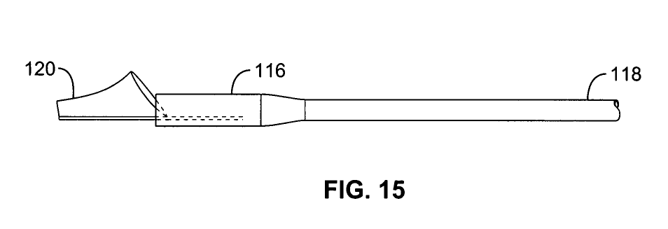

FIG. 15 through FIG. 17 illustrates another delivery system for an aortic flow diverter which does not require an introducer sheath that extends into the renal artery region of the aorta. In FIG. 15, the distal portion 116 of a delivery sheath 118 is enlarged to a diameter larger than the body of delivery sheath 118. The enlarged distal portion 116 is made of a suitable flexible material such as Pebax. Aortic flow diverter 120 is configured to fit within enlarged distal portion 116 in a collapsed or partially collapsed state.

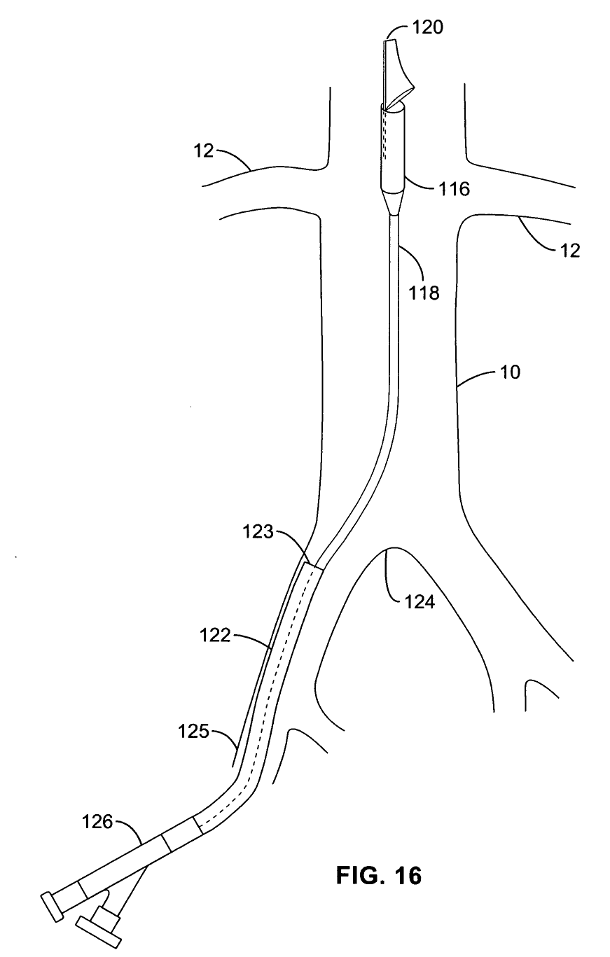

FIG. 16 illustrates schematically the positioning of the aortic flow diverter 120 shown in FIG. 15 above the renal arteries 12 in aorta 10. Introducer sheath 122 with distal end 123 is of a length to just reach the aorto-iliac bifurcation 124 from a percutaneous entry point 125. In one exemplary embodiment, the introducer sheath 122 is about 1 French larger in diameter than standard introducer sheaths. By way of comparison, sheath-within-a-sheath systems require a significant increase in introducer sheath diameter of about 3 French or more. Delivery sheath 118 is advanced through proximal coupler assembly 126 and through introducer sheath 122. Enlarged distal portion 116 of delivery sheath 118, with aortic flow diverter 120 in a partially collapsed state, is positioned just above renal arties 12 in aorta 10.

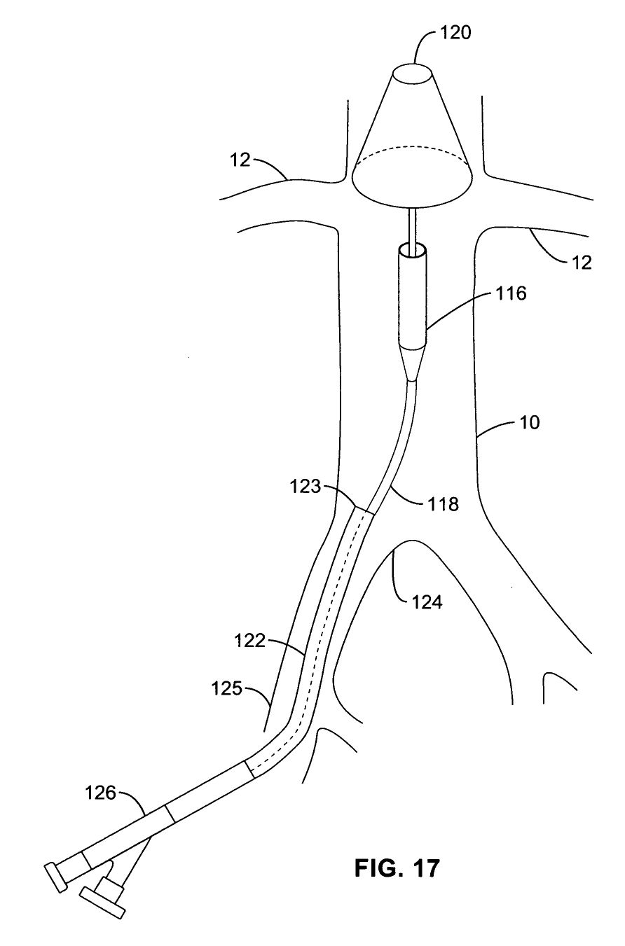

FIG. 17 illustrates the aortic flow diverter 120 in FIG. 16 where the delivery sheath 118 has been retracted through proximal coupler assembly 126 and aortic flow diverter 120 is deployed from enlarged distal portion 116 of delivery sheath 118 and assumes an expanded state at renal arteries 12. In one beneficial embodiment, delivery sheath 116 remains in the aorta system and is available to reposition aortic flow diverter 120 during medical procedures. Proximal coupler assembly 126 is not retracted to correspondingly retract the distal end 123 of introducer sheath 122. By not retracting proximal coupler assembly 126, a standard length catheter, such as 100 cm, (not shown) can be deployed through proximal coupler assembly 126 alongside delivery sheath 118 and through aortic flow diverter 120 to reach target areas (not shown) in the aorta 10 system.

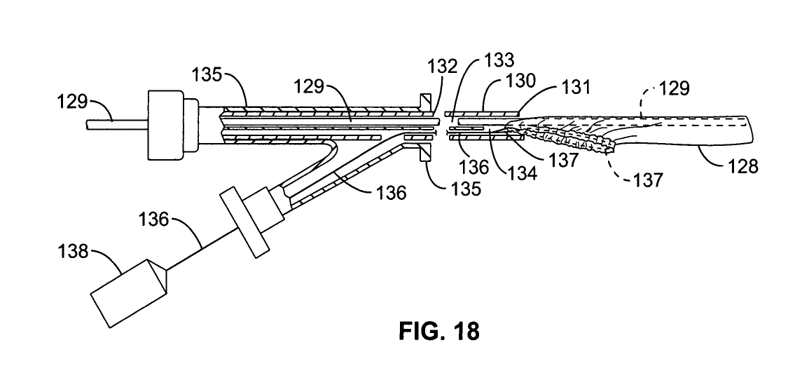

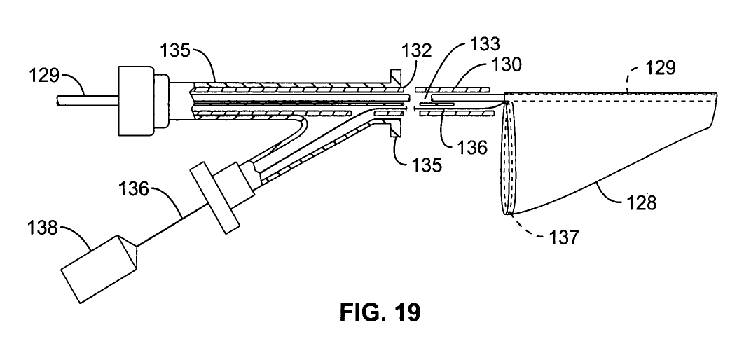

FIG. 18 and FIG. 19 are a partial cut away section views of another embodiment of an aortic flow diverter delivery system that is deployed without retracting an introducer sheath. Proximal hub assemblies for introducing a catheter have been omitted for clarity. FIG. 18 illustrates an aortic flow diverter 128 in a partially collapsed state supported on hypotube 129 which is used for structural support and fluid delivery. Delivery sheath 130 with distal end 131 and a proximal position 132 has hypotube lumen 133 and pull wire lumen 134 Proximal position 132 of delivery sheath 130 is coupled to a Y manifold assembly 135. A pull wire 136 extends from the ends of lower hoop 137 of flow diverter 128 through pull wire lumen 134 and through Y manifold assembly 135 to pull wire activator 138. Lower hoop 137 is in a hoop channel of the fabric of aortic flow diverter 128 and is not attached to hypotube 129. When pull wire activator 138 is retracted, pull wire 136 retracts lower hoop 137 of aortic flow diverter 128 partially out of the hoop channel and causes aortic flow diverter 128 to take a partially collapsed state. In one embodiment, hypotube lumen 133 and lower hoop 137 are made from Nitinol™. Further, hypotube lumen 133 and pull wire lumen 134 at distal end 131 of delivery tube 130 are adapted to accommodate the ends of lower hoop 137 when pull wire 136 is retracted.

In FIG. 19, pull wire activator 138 is relaxed and pull wire 136 advances in the pull wire lumen 134 of delivery lumen 130 allowing lower hoop 137 to expand in the hoop channel to a fully deployed state.

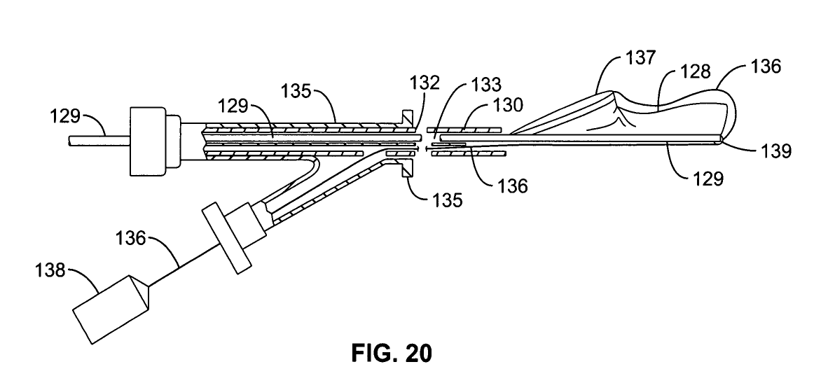

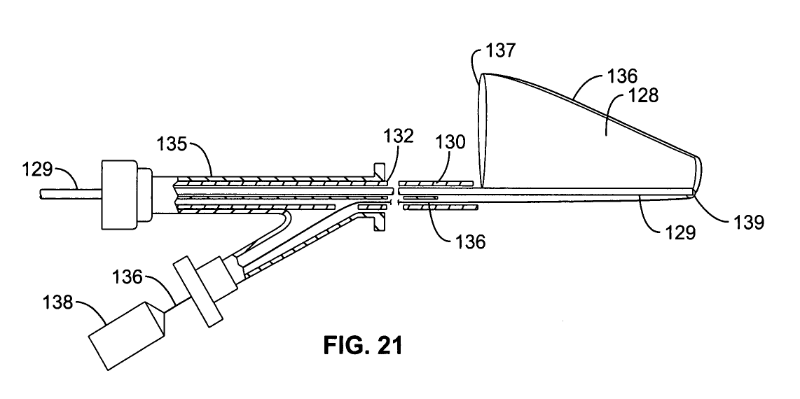

FIG. 20 and FIG. 21 are partial cut away section views of another embodiment of the aortic flow diverter delivery system shown in FIG. 18 and FIG. 19. In FIG. 20, aortic flow diverter 128 includes a pulley assembly 139 on the distal end 140 of hypotube 129. When pull wire 136 is retracted by pull wire activator 138, pull wire 136 pulls lower hoop 137 distally and flow diverter 128 assumes a collapsed or partially collapsed state.

FIG. 21 illustrates the aortic flow diverter 128 shown in FIG. 19 deployed in an expanded state by relaxing pull wire 136 and allowing lower hoop 137 to deploy proximally and expand outward.

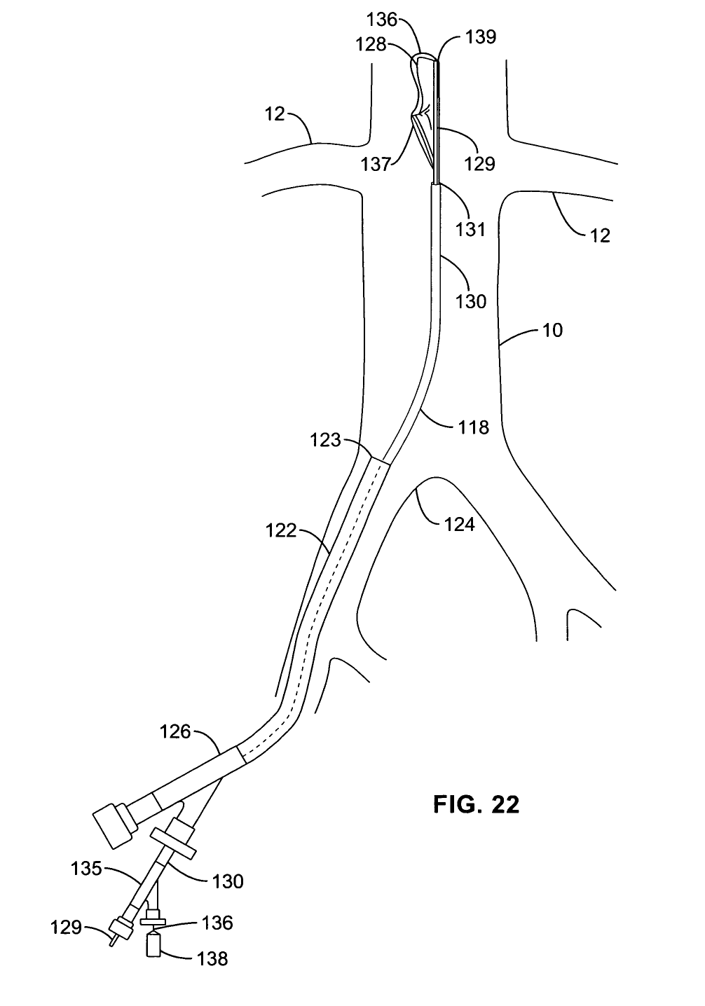

FIG. 22 illustrates an embodiment of the aortic flow diverter 128 in FIG. 21 with a proximal hub assembly 126 and introducer sheath 122 as shown in FIG. 16 and FIG. 17. Proximal hub assembly 126 couples Y hub assembly 135 and introducer sheath 122. Delivery tube 130 is advanced through Proximal hub assembly 126 through introducer sheath 122 until distal end 131 is in the region of renal arteries 12. Pull wire 136 is retracted pulling lower hoop 137 up to partially collapse aortic flow diverter 128. Delivery sheath 130 is advanced to position and deploy aortic flow diverter 128. When pull wire 136 is relaxed, aortic flow diverter 128 expands and deploys. Delivery sheath 130 can be retracted during deployment without retracting introducer sheath 122. A standard interventional catheter (not shown) may be advanced through proximal coupler assembly 126 and through introducer sheath 122 along side delivery sheath 130.

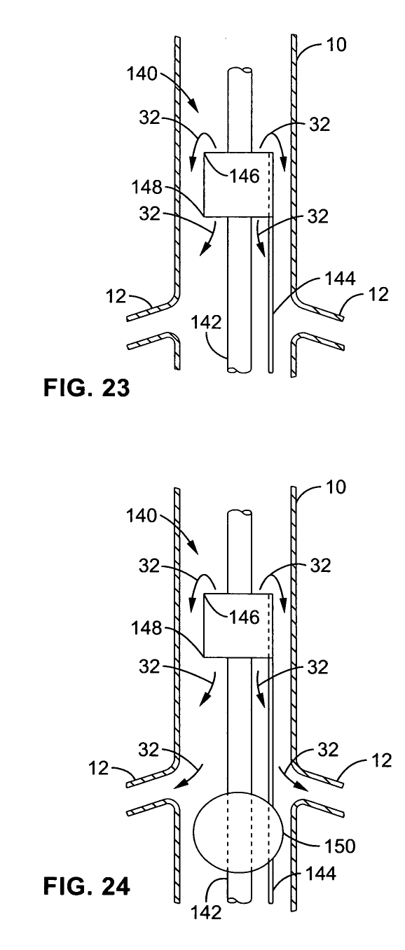

FIG. 23 shows schematically an aortic flow diverter 140 configured as a collar around guide catheter 142 and supported by fluid delivery lumen 144. Aortic flow diverter 140 has a distal hoop 146 and a proximal hoop 148. Infusion ports (not shown) are positioned on the inside of aortic flow diverter 140 and fluidly connected to fluid delivery lumen 144. Distal hoop 146 of and proximal hoop 148 slide on guide catheter 142. In this example, fluid agent 32 perfuses out distal hoop 146 and proximal hoop 148 of aorta flow diverter 140 and to the lower extremities including renal arteries 12. It is to be understood that additional variations (not shown) of aortic flow diverters are contemplated. In one embodiment, aortic flow diverter 140 is configured with the distal hoop 146 adapted to slide closely to guide catheter 142 to preferentially perfuse fluid agent out the proximal hoop 148. In another embodiment, aortic flow diverter 140 is configured with the proximal hoop 148 adapted to slide closely to guide catheter 142 to preferentially perfuse fluid agent out the distal hoop 146. It is to be understood that aortic flow diverter 140 can be configured with both hoops 146, 148 loosely adapted to perfuse fluid agent from both hoops 146, 148.

FIG. 24 shows another embodiment of aortic flow diverter 140 in FIG. 23 where an expandable tubular member 150 is coupled to fluid delivery lumen 144 and positioned proximal of aortic flow diverter 140. In this embodiment, aortic flow diverter 140 is positioned upstream of renal arteries 12 and expandable tubular member 150 is positioned below renal arteries 12 in aorta 10 to divert blood flow preferentially toward the renal arteries 12. Fluid agent 32 perfuses out the distal hoop 148 of aorta flow diverter 140 and preferentially into renal arteries 12.

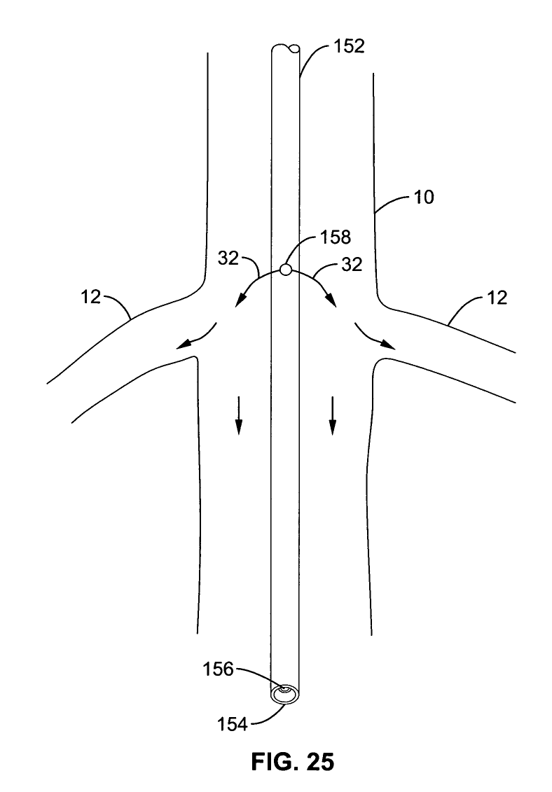

FIG. 25 illustrates schematically a fluid agent delivery catheter where catheter 152 is a dual lumen extrusion with one large lumen 154 for interventional equipment and a small lumen 156 for fluid agent delivery. Catheter 152 has fluid agent port 158 that is fluidly connected to small lumen 156. Catheter 152 is positioned in aorta 10 with fluid agent port 158 upstream of renal arteries 12 for delivery of fluid agent 32 to renal arteries 12. in this configuration, approximately 15 percent of the fluid agent 32 infused from fluid agent port 158 reaches each renal artery 12 for a total of 30 percent. This embodiment has the advantage of eliminating a second fluid agent delivery device.

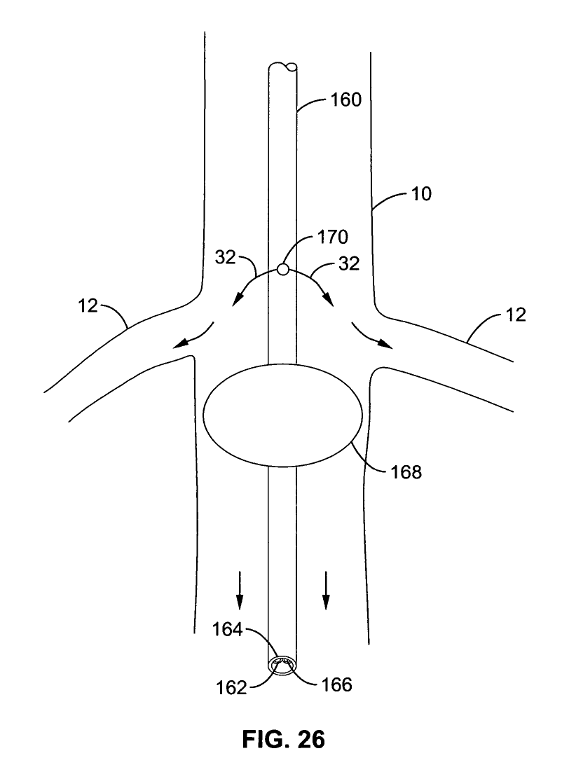

FIG. 26 illustrates schematically a fluid delivery catheter similar to the one shown in FIG. 25 where catheter 160 comprises three lumens; a large lumen 162 for interventional equipment, a first small lumen 164 for fluid agent delivery, and a second small lumen 166 for inflation. A radially inflatable member 168 is attached to catheter 160 proximal of fluid agent port 170. First small lumen 164 is fluidly connected to fluid agent port 168. Second small lumen 166 is fluidly connected to radially inflatable member 168. Radially inflatable member 168 may be made from a compliant or semi-compliant material such as nylon, PEBAX, polyurethane or silicone. Lumen 160 is positioned into aorta 10 with fluid agent port 170 upstream of renal arteries 12 and radially inflatable member 168 downstream of renal arteries 12. Radially inflatable member 168 is inflated to partially or completely block aortic blood flow and increase blood flow into the renal arteries 12. Fluid agent is perfused from fluid agent port 170 into the aortic blood flow. This embodiment has the advantage of delivering more fluid agent 32 to the renal arteries 12 due to the flow diversion of radially inflatable member 168.

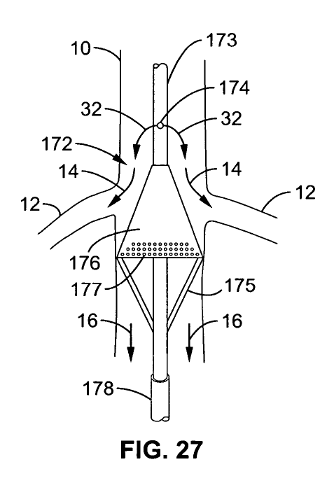

FIG. 27 illustrates flow diverter assembly 172 coupled to catheter 173 at a position proximal of fluid delivery port 174 in catheter 173. A frame 175, configured much like a basket or an umbrella, supports membrane 176. The frame 175 is preferably made from a memory metal, e.g., NiTi, to allow for conformability to the aorta and pre-shaped capabilities. In this aspect of the present invention, the membrane 176 can be made from nylon, PEBAX, polyurethane, low density PTFE or any other similar material with low porosity to allow for blood diffusion through the membrane 176. Moreover, the membrane 176 can be lazed or otherwise formed with plural holes 177 of varying diameter, e.g., from twenty-five micrometers to five-hundred micrometers (25 μm-500 μm) to allow blood flow through the material film. It can be appreciated that the flow diverter 172 can be expanded such that it engages the inner wall of the abdominal aorta 10. Further, flow diverter 172 can be collapsed within an outer sheath 178 disposed around the drug infusion catheter 173. Once the drug infusion catheter 173 is in place within the abdominal aorta 10, the sheath 178 can be retracted causing the flow diverter 172 to be deployed in the region of the renal arteries 12.

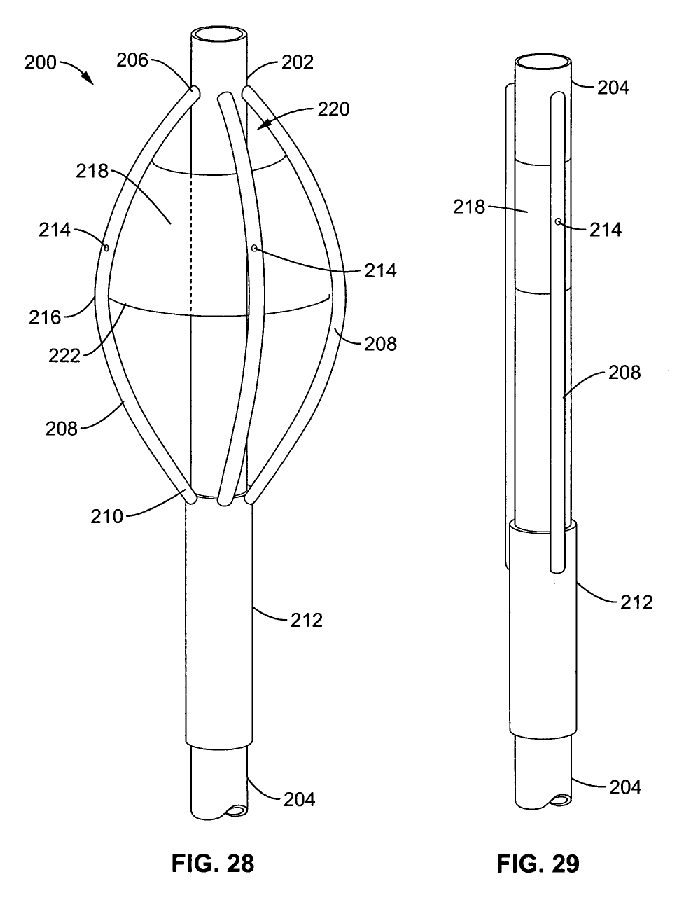

FIG. 28. and FIG. 29 illustrate an expandable aortic flow diverter 200 placed near the distal end 202 of multi lumen catheter 204. In one beneficial embodiment, catheter 204 is a specialized introducer sheath/infuser type of about 6 French to about 8 French in diameter. The distal ends 206 of three or more flexible, hollow struts 208, made of suitable shape retaining material such as Nitinol™ hypotubing, are fluidly connected near the distal end 202 of catheter 204 It is understood that other arrangements for fluidly coupling struts 208 to multilumen catheter 204 may be used and that the struts 208 may be of flattened tubing. FIG. 28 illustrates a beneficial embodiment with three struts 208 visible. The proximal ends 210 of struts 208 are connected to the distal end of a diverter sheath 212 proximal of aortic flow diverter 200. Struts 208 assume a bow shape parallel to catheter 204 when deployed. An infusion port 214 is placed in the wall of each strut 208 distal of the bow apex 216 of struts 208 by a suitable process such as a laser cut hole, slit or other micro fenestration process. Membrane 218 is a stretchable fabric formed in a truncated cone or funnel shape and attached to struts 208 with the smaller opening 220 of membrane 216 attached near the distal end 206 of struts 208 forming an annular opening around catheter 204. The larger opening 222 of membrane 216 is attached near the bow apex 214 of struts 208. When diverter sheath 212 is advanced distally on catheter 204, aortic flow diverter 200 is expanded outward.

FIG. 29 illustrates the expandable aortic flow diverter 200 shown in FIG. 28 in a collapsed state with diverter sheath 212 retracted proximally on catheter 204, struts 208 straightened, and membrane 218 collapsed against catheter 204.

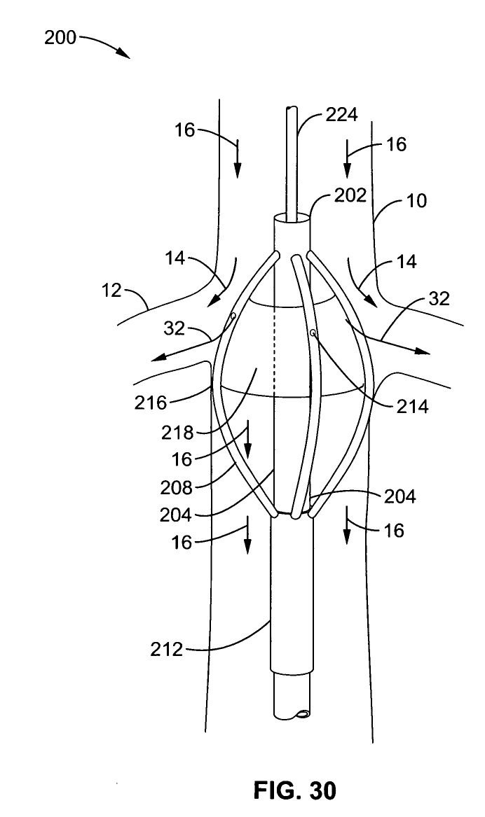

FIG. 30 is a schematic illustration of the expandable aortic flow diverter 200 shown in FIG. 28 positioned in aorta 10 to infuse a fluid agent 32 into renal arteries 12. Distal end 202 of catheter 204 is positioned above renal arteries 12. Diverter sheath 212 is advanced distally on catheter 204 allowing the bow apex 216 of struts 208 to contact the inner wall of aorta 10. Membrane 218 diverts outer aortic blood flow 14 into renal arteries 12. Fluid agent 32 is infused from infusion ports 214 and into the renal arteries 12. Having multiple infusion ports 214 eliminate the need to rotate aortic flow diverter 200 for correct positioning. Inner blood flow 16 flows through the annular space between membrane 218 and catheter 204 and down aorta 10 to the lower extremities. Guide catheter 224 is deployed upstream through catheter 204 for further intervention procedures.

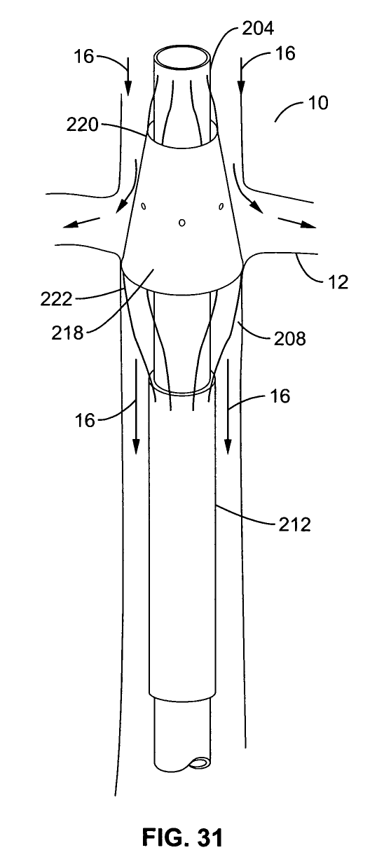

FIG. 31 is a stylized illustration of the expandable aortic flow diverter 200, shown in FIG. 28, adapting to a small aorta 10. It is understood that there are a number of different ways of positioning infusion ports 214 on the outside of membrane 218, or supporting membrane 218. Upper edge 220 of membrane 218 forms a relatively smaller annular space when the lower edge 222 of membrane 218 is sealed against the inner wall of aorta 10.

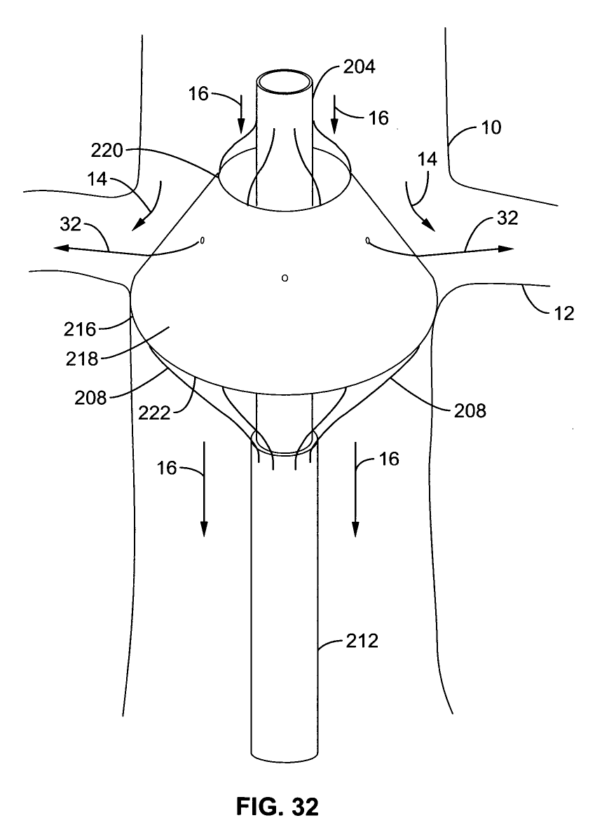

FIG. 32 shows the expandable aortic flow diverter 200 shown in FIG. 28 adapting to a large aorta 10 where upper edge 220 of membrane 218 forms a relatively larger annular space when the lower edge 222 of membrane 218 is sealed against the inner wall of aorta 10.

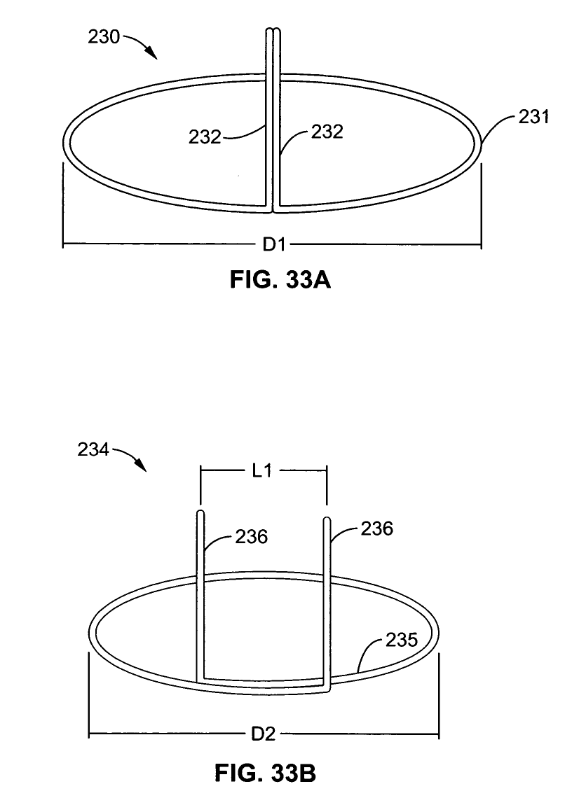

FIG. 33A through FIG. 36 illustrate a beneficial adaptation for a wire hoop in an aortic flow diverter. Because aortic flow diverters are typically compressed in a sheath to advance in the aorta and position near the renal arteries, as discussed previously, the wire hoops of the aortic flow diverter may experience a permanent kink if the superelastic limit of the wire material is exceeded in the compressed state. The relative tendency to kink increases as the hoop diameter relative to the sheath size increases or the wire diameter increases.

FIG. 33A illustrates a typical wire hoop 230 formed for an aortic flow diverter with hoop element 231 having diameter D1. In this embodiment, legs 232 are formed at a 90 degree angle from the hoop element 231 and legs 232 are close together or touching when wire hoop 230 is in it free state. This is the at rest configuration of a typical wire hoop 230 when integrated into an aortic flow diverter.

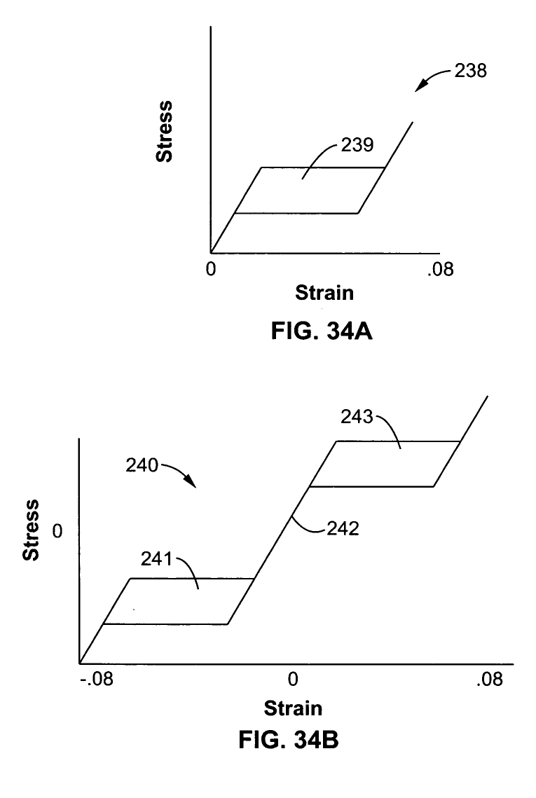

FIG. 33B illustrates a wire hoop 234 formed with hoop element 235 having diameter D2. Legs 236 are formed at a 90 degree angle from the hoop element 235 and legs 236 are spaced apart by length L1 when wire hoop 234 is in its free state. D2 in FIG. 33B is smaller than D1 in FIG. 33A, but, in this example, wire hoop 234 increases to a diameter about equal to D1 when legs 236 are brought close together or touch. When wire loop 234, shown in FIG. 33B, is used in a flow diverter and compressed in a sheath, it has a decreased tendency to kink than a comparable wire loop 230, as shown in FIG. 33A, made of similar diameter and material. This decreased tendency to kink is further explained below and in FIG. 34A and FIG. 34 B. In an exemplary embodiment, a wire hoop 234 is made of Nitinol™ wire of about 0.014 inch diameter but wire diameters of 0.011 inches and about 0.013 inches are contemplated. The diameter of hoop element 235 in its free state is about 19.8 millimeters and the diameter in its expanded state when the legs 236 are brought together is about 22.9 millimeters. However, hoop diameters in the expanded state of about 20 millimeters to about 25 millimeters are contemplated. In this embodiment, the wire hoop 235 can be collapsed into an introducer sheath of about 8 French nominal diameter without permanent deformation.

FIG. 34A is illustrative of the stress strain relationship 238 for wire hoop 230 in FIG. 33A and FIG. 34A is illustrative of the stress strain relationship 240 for wire hoop 234 in FIG. 33B. In FIG. 34A, the rhombus area 239 of relationship 238 represents a region where a hoop of memory shape material, such as Nitinol™ wire, will return to its free state when the stress of compression is reduced, and in this embodiment, eventually to zero. In this non limiting example, wire hoop 230 will not kink in a range from zero to about region 239. A linear compressive strain beyond region 239 or in this example, greater than about 8 percent, results in permanent deformation, or kinking of the wire hoop 230.

FIG. 34B illustrates the stress strain relationship 240 for wire hoop 234 in FIG. 33B. Wire hoop 240 is first expanded from a free state as shown in FIG. 33B to a form similar to hoop 230 in FIG. 33A and integrated into a flow diverter (not shown). This expanded state is expressed as a negative stress represented by negative strain region 241. When wire hoop 234 is compressed, it first returns to a zero stress, zero strain state 242, then continues into a compressive strain region 243. The range of non deforming stress and strain, from region 241 to region 243, in this example is about double the range of zero to region 239 shown in FIG. 34A.

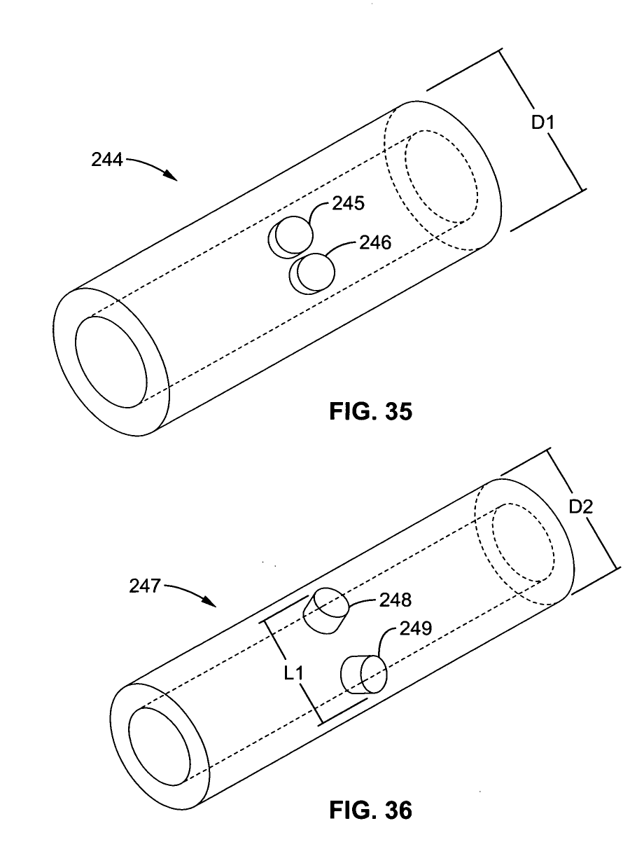

FIG. 35 illustrates a tool for producing a wire hoop similar to wire hoop 230 in FIG. 33A. Cylindrical forming mandrel 244, of diameter D1 as shown in FIG. 33A, has axis pins 245 and 246 positioned on the cylindrical surface of mandrel 246 perpendicular to the longitudinal axis of mandrel 246 and relatively close together. A wire hoop 230 is formed by looping the wire around mandrel 244 to form hoop element 231 and pulling the ends of the wire between pins 245, 246 to form parallel legs 232 as shown in FIG. 33A.

FIG. 36 illustrates a tool for producing wire hoop 234 in FIG. 33B. forming mandrel 247 has axis pins 248 and 249 positioned perpendicular to the longitudinal axis of mandrel 246 and apart at predetermined distance about L1 relative to each other. A wire hoop 234 is formed by looping the wire around mandrel 247 and pulling the wire ends past the outside of pins 248, 249 relative to each other, and then perpendicular to form parallel legs 236 as shown in FIG. 33B In one beneficial embodiment, mandrel 246 is about 0.75 inches in diameter and pins 248 and 249 form an angle of about 43 degrees when projected through the centerline of mandrel 246. In a further beneficial embodiment, wire hoop 234 is positioned tightly on mandrel 246 as described above and placed in a furnace at 535 degrees centigrade for 10 minutes.

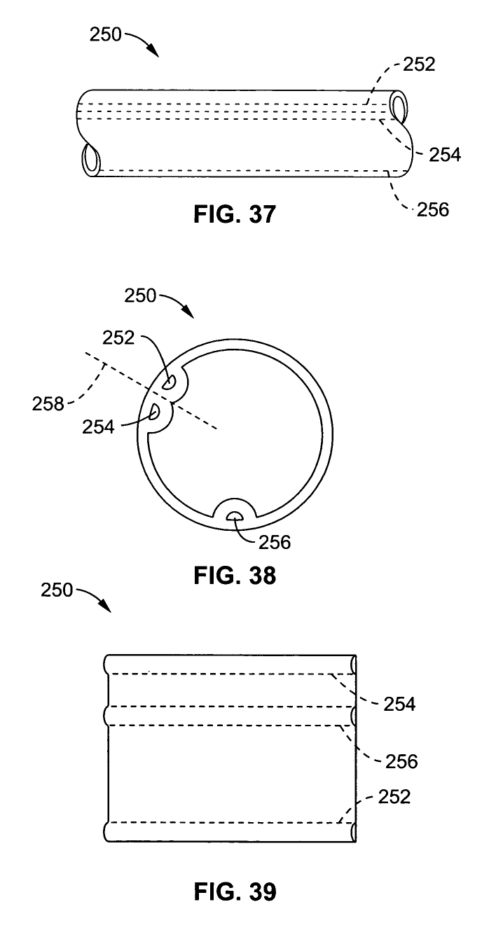

FIG. 37 through FIG. 39 illustrate steps for creating a sheet material with integrated lumens or channels for further assembly into an aortic flow diverter that is beneficial for process and bulk considerations in relation to assembly from sheet material. For clarity and understanding, a typical method of manufacturing of a flow diverter is described first without illustration. In one mode, manufacturing starts with sheet or fabric ePTFE cut in a rhombus shaped template (not shown). Channels at the edge of the fabric are made by rolling the material over a mandrel and bonding with silicone or a suitable bonding agent. A third infusion channel about midway in the fabric requires bonding another piece of ePTFE to the main sheet with silicone or other suitable bonding agent. This process is relatively complex, time consuming and increases bulk in the resultant aortic flow diverter which is typically compressed into about an 8 French diameter sheath.

In FIG. 37, a highly beneficial method is described where tube 250 is formed by ram extruding a slurry of PTFE powder and solvent. The resultant ePTFE properties are determined by extrusion parameters and post processing (not shown). Tube 250 is extruded forming multiple lumens, and in this non-limiting embodiment, three lumens 252, 254 and 256 respectively are formed. FIG. 38 is a cross section of the tube 250 shown in FIG. 37 showing the position of lumens 252, 254 and 256 and the position of axial cut line 258.

FIG. 39 illustrates tube 250 shown in FIG. 38 flattened into a sheet after axial cut 258, typically with a calendaring process. Lumen 252 and lumen 254 are positioned on the top and bottom edge respectively to mount on a wire hoop or other support to form an aortic flow diverter. Lumen 256 is positioned between lumen 252 and lumen 254 in the sheet to form a channel to connect to a support tube and infusion ports in an aortic flow diverter

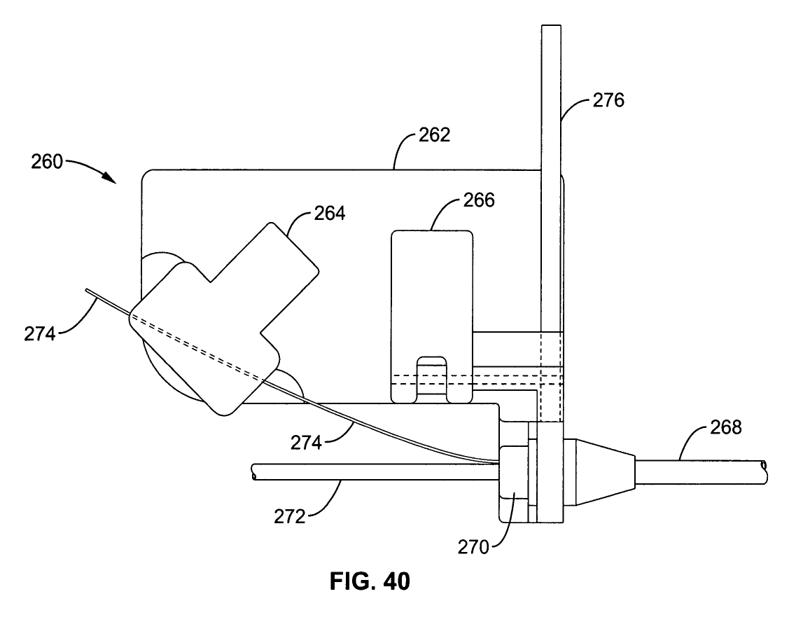

FIG. 40 illustrates an aortic flow diverter clip assembly 260 for insertion and positioning of aortic flow diverters adjunctive with catheters and other medical devices. It is to be understood that aortic flow diverter clip assembly 260 may be used for insertion and positioning of other devices adjunctive with a catheter. Details of manipulation handles, pivot pins and springs are omitted for clarity. Clip assembly 260 comprises a base 262, configured to accommodate an infusion line clip 264 and a hemostasis valve clamp 266. A typical introducer sheath 268 terminates at hemostasis valve assembly 270 which is held in position by hemostasis valve clamp 266. Guide catheter 272 and infusion lumen 274 enter introducer sheath 268 through hemostasis valve assembly 270. While guide catheter 272 enters hemostasis valve assembly 270 in an approximately straight position, infusion lumen 274 is guided in a gentle curve towards hemostasis valve assembly 270 and held in position by infusion line clip 264. A side port tube 276, for infusion of saline solution, or other fluid agent, into introduction sheath 268 is shown connected to hemostasis valve assembly 270 and positioned under hemostasis valve clamp 266.

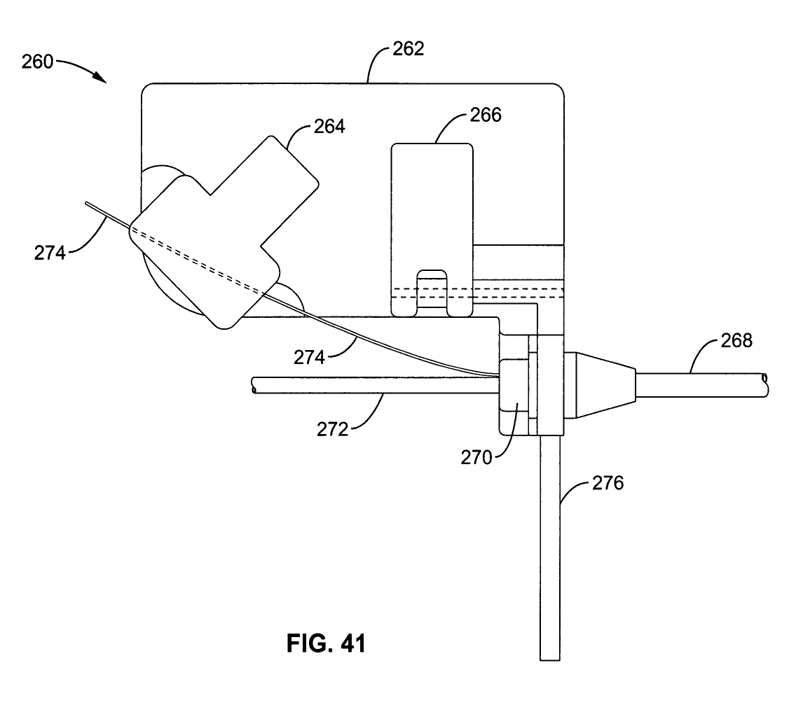

FIG. 41 is another embodiment of the clip assembly 260 in FIG. 40 with side port tube 276 connected to hemostasis valve assembly 270 and positioned opposite hemostasis valve clamp 266.

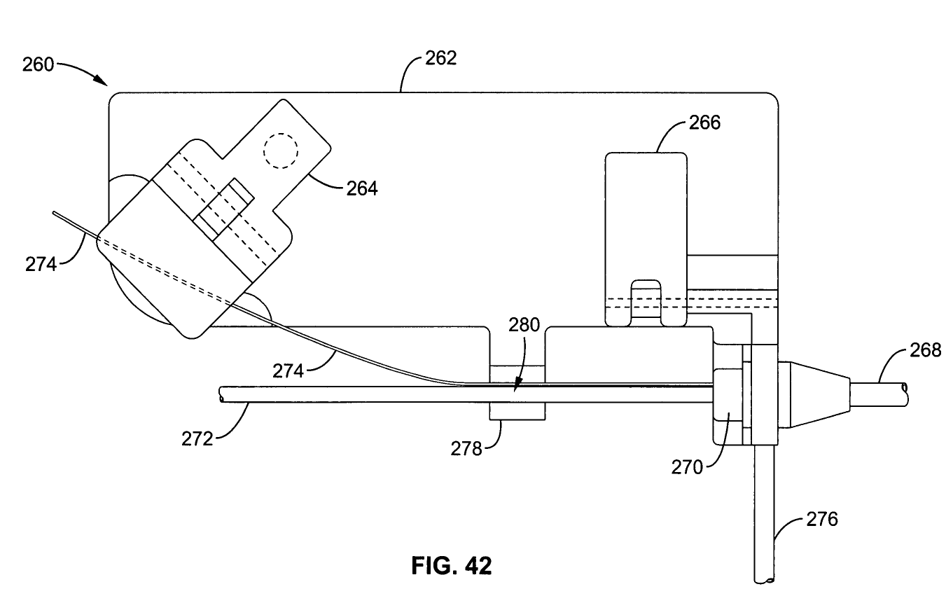

FIG. 42 illustrates another beneficial embodiment of the flow diverter clip assembly 260 shown in FIG. 40 where bracket 278 is adapted to base 262 approximately medial of infusion line clip 264 and hemostasis valve 270 with channel 280 configured to hold infusion lumen 274 in a straightened position and adjacent guide lumen 272. Infusion lumen 274 is guided in a gentle curve toward bracket 278 and held in position by infusion line clip 264. This embodiment reduces potential for leakage at hemostasis valve 270 due to deflection of infusion lumen 274.

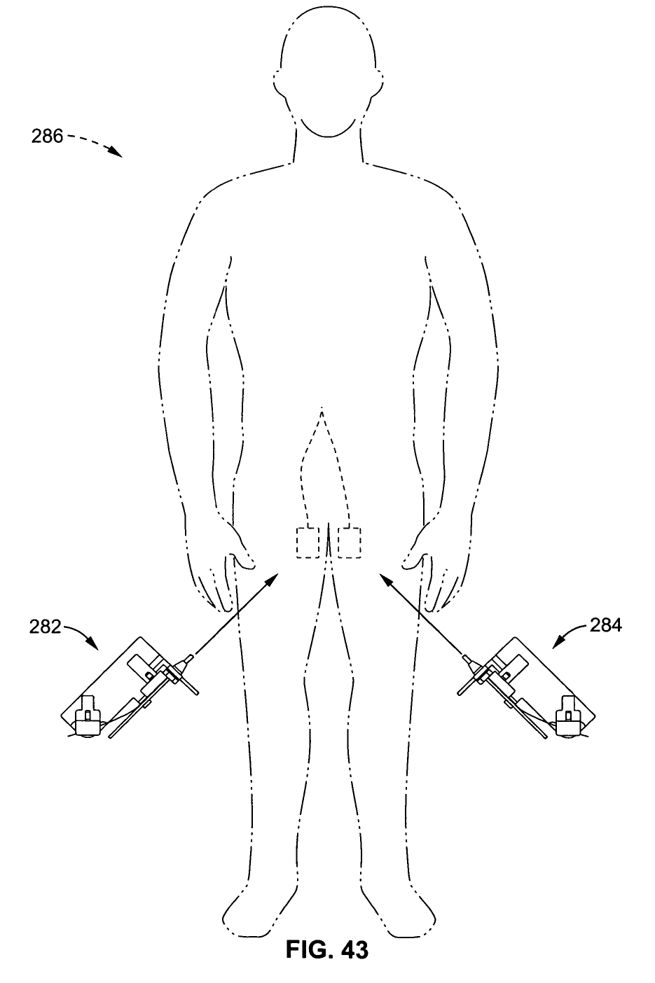

FIG. 43 illustrates the positioning of a left flow diverter clip assembly 282, with the infusion tube exiting to the left, and a right flow diverter clip assembly 284, with the infusion lumen exiting to the right. Actual selection and placement of a left or right diverter clip assembly 282, 284 depends on the intervention procedures on patient 286 and physician preference.

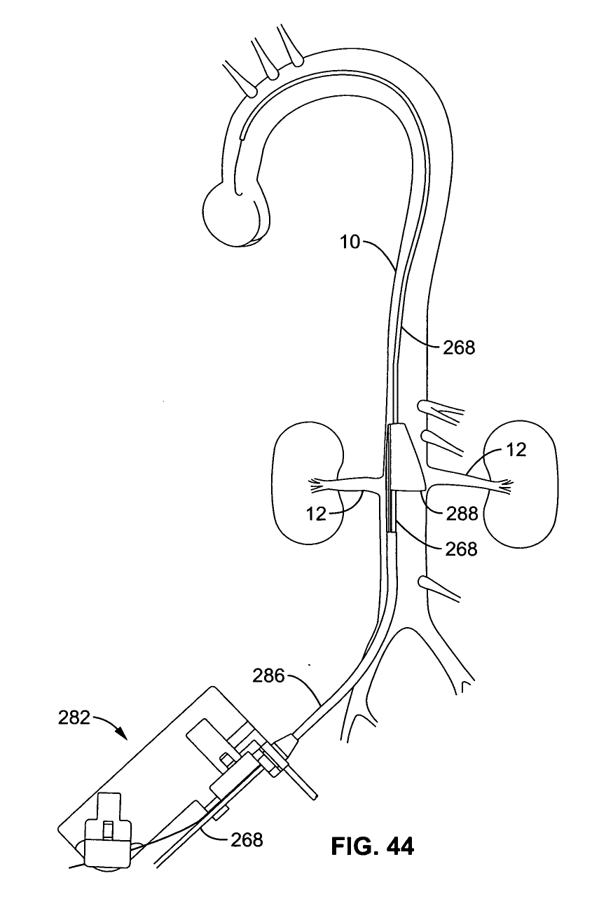

FIG. 44 illustrates the position of a left flow diverter clip assembly 282 shown in FIG. 43 coupled to introducer sheath 286 inserted in the common iliac artery with guide catheter 268 in the upper portion of aorta 10 and aortic flow diverter 288 positioned near renal arteries 12. Left diverter clip assembly 282 anchors aortic flow diverter 288 in place during manipulation of guide catheter 268.

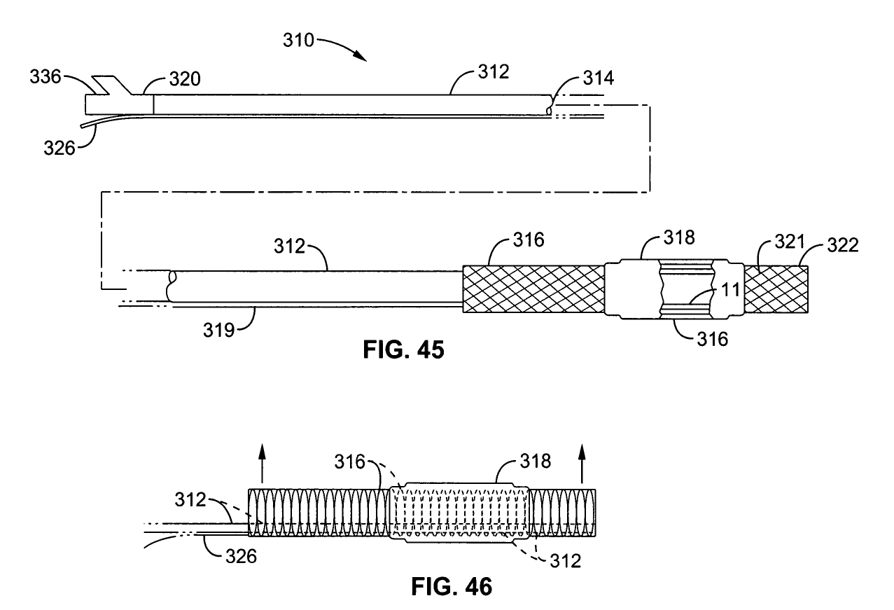

FIG. 45 through FIG. 50 illustrate an aortic flow diverter 310 generally comprising an elongated shaft 312 having a proximal end, a distal end, and at least one lumen 314 extending therein, a tubular member 316 on a distal section of the elongated shaft 312 and a radially expandable member 318 on the tubular member 316. Adapter 320 on the proximal end of the shaft provides access to lumen 314. FIG. 45 illustrates the tubular member 316 and the radially expandable member 318 in low profile, unexpanded configurations for entry into the patient's blood vessel.

In FIG. 45, the radially expandable member 318 on flow diverter 310 is an inflatable balloon. The radially expandable member 318 has proximal and distal ends secured to an outer surface of the tubular member 316, and an interior in fluid communication with an inflation lumen 328 (shown in FIG. 48) in the shaft 312. The radially expandable member 318 can be formed of a variety of suitable materials typically used in the construction of catheter occlusion balloons, and in another embodiment is highly compliant and is formed of a material such as latex, polyisoprene, polyurethane, a thermoplastic elastomer such as C-Flex. In another embodiment, the radially expandable member 318 may be noncompliant or semi-compliant. While discussed primarily in terms of a radially expandable member comprising a balloon, it should be understood that the radially expandable member may have a variety of suitable configurations.

In FIG. 45, the tubular member 316 comprises braided filaments 321, such as wire, ribbon, and the like, having a sheath 322, and having a lumen or interior passageway 324 (shown in FIG. 49) therein. A pull line 326 having a distal portion secured to the tubular member is configured to be retracted or pulled proximally to radially expand the tubular member 316. Specifically, the braided filaments 321 can reorient from a longer, smaller diameter configuration and a shorter, larger diameter configuration cause the tubular member 316 to shorten, thereby radially expanding the tubular member 316. When the pull line 326 is not under tension, the spring force of the elastomeric material of the sheath 322 will cause the tubular body 316, defined by the braided filaments 321, to elongate and reduce in diameter. The sheath 322 is preferably an elastomeric polymer on the braided filaments. The sheath 322 can be on an inner or outer surface of the braided filaments 321, or the braided filaments 321 can be completely or partially embedded within the sheath 322. In the embodiment in which the sheath 322 is on a surface of the braided filaments 321, the sheath 322 is preferably secured to a surface of the filaments 321 as for example with adhesive or heat bonding. The braided filaments 321 can be formed of a variety of suitable materials such as metals or stiff polymers. A variety of suitable polymeric materials can be used to form the sheath 322. While discussed below primarily in terms of a tubular member comprising a braided tube, it should be understood that the tubular member may have a variety of suitable configurations.

The dimensions of catheter 310 are determined largely by the size of the blood vessel(s) through which the catheter must pass, and the size of the blood vessel in which the catheter is deployed. In a beneficial embodiment, the length of-the-tubular member 316 is about 50 to about 150 mm, preferably about 80 to about 120 mm. The tubular member 316 has an unexpanded outer diameter of the tubular member of about 1 mm to about 5 mm, preferably about 2 to about 4 mm, and a radially expanded outer diameter of about 40 mm to about 140 mm, preferably about 60 mm to about 120 mm. The radially expanded interior passageway 324 of the tubular member 316 is about 30 mm to about 130 mm, preferably about 50 mm to about 110 mm to provide sufficient perfusion. The interior passageway 324 of the tubular member 316 has a radially expanded inner diameter which is about 1000% to about 6000% larger than the unexpanded inner diameter of the passageway 324. The radially expandable member 318 has a length of about 10 mm to about 50 mm, preferably about 20 mm to about 40 mm. The expanded outer diameter of the radially expandable member 318 is about 10 mm to about 35 mm, preferably about 15 mm to about 30 mm. In this embodiment, the shaft 312 has an outer diameter of about 1 mm to about 5 mm. The inflation lumen 328 (shown in FIG. 48) has an inner diameter of about 0.02 mm to about 0.06 mm and the agent delivery lumen 332 (shown in FIG. 48) has an inner diameter of about 0.01 mm to about 0.04 mm. The length of the shaft 312 is about 40 mm to about 100 cm, but in a further beneficial embodiment, about 60 to about 90 cm.

FIG. 46 illustrates the tubular member 316 in the expanded configuration after retraction of the pull line 326. As best illustrated in FIG. 46, showing the distal section of the shaft 312 within the inner lumen of the tubular member 316 in dotted phantom lines, the distal end of the shaft 312 is located proximal to the distal end of the expanded tubular member 316. In the embodiment illustrated in FIG. 46, the radially expandable member 318 is in a non-expanded configuration. The section of the expanded tubular member 316 under the radially expandable member 318 is illustrated in dashed phantom lines.

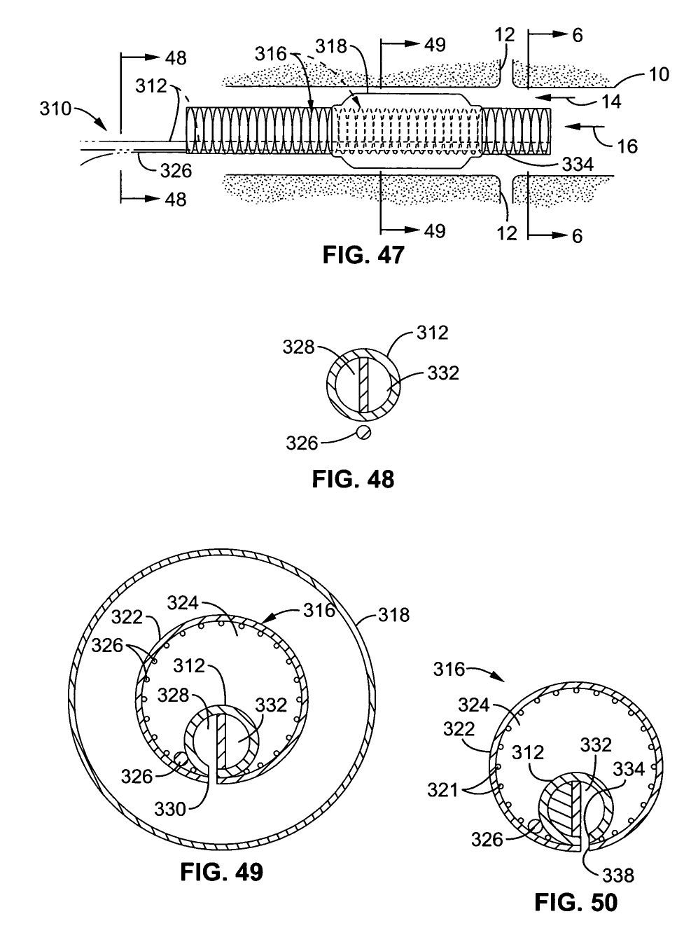

FIG. 47 illustrates schematically, the expanded tubular member 316 with the radially expandable member 318 in the expanded configuration. As best illustrated in FIG. 48, FIG. 49 and FIG. 50 showing transverse cross sections of the elongated shaft 312 shown in FIG. 47, taken along lines 48-48, 49-49, and 50-50, respectively, the elongated shaft 312 has an inflation lumen 328 extending from the proximal end of the shaft 312 to an inflation port 330 (shown in FIG. 49) located on the shaft distal section, in fluid communication with the interior of the radially expandable member 318. Arm 336 on adapter 320 (shown in FIG. 45) provides access to the inflation lumen 328, and is in fluid communication with a source of inflation fluid (not shown). The elongated shaft 312 also has an agent delivery lumen 332 extending from the proximal end to an agent delivery port 334 in the distal end of the shaft 312. Arm 336 on adapter 320 (shown in FIG. 45) provides access to the agent delivery lumen 332, and is in fluid communication with an agent source (not shown). The tubular member sheath 322 has an agent delivery opening 338 adjacent to the shaft agent delivery port 334, for providing a pathway for agent delivery from the lumen 332 to exterior to the tubular member 316. In the illustrated embodiment, the inflation lumen 328 and agent delivery lumen 332 are side-by-side in a multilumen shaft 312, with inflation port 330 extending through a side wall of the shaft 312, as shown in FIG. 48. However, a variety of suitable configurations may be used as are conventionally used in catheter shaft design including coaxial lumens in fluid communication with side ports or ports in the distal extremity of the shaft. The agent delivery port 334 is preferably in a side wall of the shaft 312 distal section in fluid communication with the agent delivery lumen 332, however, alternatively, the agent delivery port 334 may be in the distal end of the shaft 312.

These embodiments are illustrated schematically and the relationship of the elements may be combined in various combinations and specific modes by one of ordinary skill in the art. For example, FIG. 49 illustrates a more specific embodiment where multilumen shaft 312 is attached to the inner wall of tubular member 316. Inflation lumen 328 is in fluid communication through inflation port 330 and agent delivery lumen 332 is in fluid communication with blood flow 14 through agent delivery port 334 and agent delivery opening 338.

FIG. 47 illustrates the catheter 310 in a descending aorta 10, of a patient, having renal arteries 12, opening therein. The catheter 310 is introduced and advanced within the patient's blood vessel 10 in the low profile, unexpanded configuration shown in FIG. 45. The agent delivery port 334 is positioned proximate to (up-stream or inline with) the one or more branch vessels 12, and the distal end of the tubular member is preferably up-stream of the one or more branch vessels 12. The tubular member 316 is expanded to its expanded configuration, and, preferably, thereafter the radially expandable member 318 is radially expanded by directing inflation fluid into the radially expandable member 318 interior. Specifically, in one mode, the elongated shaft 312 is introduced into the femoral artery, as for example by the Seldinger technique, preferably slidingly over a guide wire (not shown), and advanced into the descending aorta 10. Although not illustrated, the elongated shaft 312 may be provided with a separate guide wire lumen, or the catheter may be advanced over a guide wire in agent delivery lumen 332 adapted to slidingly receive a guide wire. Alternatively, the catheter 310 may be advanced without the use of a guide wire. The agent delivery port 334 is positioned proximate to one or both renal arteries 12, as illustrated in FIG. 47, and the tubular member 316 extends within the aorta 12 up-stream and down-stream of the renal arteries 12. The tubular member 316 is radially expanded by retracting pull line 326. The interior passageway 324 of the tubular member 316 separates blood flow through the blood vessel 10 into an outer blood flow stream 14 exterior to the tubular member 316, and an inner blood flow stream 16 within the interior passageway 324 of the tubular member 316.

The radially expandable member 318 is expanded by directing inflation fluid into the inflation lumen 328. In the embodiment illustrated in FIG. 47, the radially expandable member 318 is expanded to an outer diameter which does not completely occlude the patient's aorta 10. However, in another mode, the balloon expands into contact with the wall of the aorta 10, to an outer diameter which completely occludes the outer blood flow 14 in aorta 10 (not shown). Radially expandable member 318 may have a length and elongated configuration configured to provide mechanical stability for and coaxial centering of the operative distal section of the catheter in the aorta 10. A stabilizing member (not shown) may be provided on an outer surface of the distal end of the tubular member 318, such as for example unfoldable arms which anchor the distal end of the catheter in the aorta 10 during delivery of agent.

A variety of suitable imaging modalities may be used to position the catheter in the desired location in the blood vessel, such as fluoroscopy, or ultrasound. For example, radiopaque markers (not shown) on the shaft 312 may be used in positioning the balloon 318 and agent delivery port 334 at the desired location in the blood vessel 10.

A therapeutic or diagnostic agent (hereafter “agent”) is delivered to the renal arteries 10 by introducing the agent into the agent delivery lumen 332 in shaft 312, and out the agent delivery port 334. An agent delivery opening 338 in the tubular member 316 adjacent to the agent delivery port 334 provides a pathway for agent delivery from lumen 332 to external to the tubular member 312. The agent delivery port 334 is up-steam of the renal arteries 12 and proximal to the distal end of the tubular member 316. Thus, the outer blood flow stream 14 has a relatively high concentration of agent and the inner blood flow stream 16 has a relatively low concentration or no agent. Additionally, the balloon 318 in the expanded configuration restricts the flow of blood to decrease the blood flow exterior to the proximal portion of the tubular member 316 down-stream of the renal arteries 12 in comparison to the blood flow stream exterior to the distal portion of the tubular member 316 up-stream of the renal arteries 12. As a result, a relatively large amount of the agent delivered from the agent delivery port 334 is directed into the renal arteries 12, in comparison to the amount of agent which flows down-stream of the renal arteries 12 in the aorta 10. In one embodiment, the outer blood flow stream 14 is substantial.

In one embodiment, the cross-sectional area of the inner lumen 324 of the tubular member 316 is about 4% to about 64% of the blood vessel 10 (i.e., aorta) cross-sectional area, or about 4 mm to about 16 mm for a blood vessel 10 having a 20 mm inner diameter. It should be noted that in some embodiments, the cross-sectional area of the wall of the tubular member 316 is not insignificant in relation to the cross-sectional area of the blood vessel 10. In the embodiment illustrated in FIG. 45 in which tubular member 316 comprises sheath 322 on a frame of filaments 321, this cross-sectional area is negligible. In one beneficial embodiment, the cross-sectional area of the wall of the tubular member 316 may be about 2% to about 50%, more specifically about 5% to about 20%, of the cross-sectional area of a section of the blood vessel 10 located at the up-stream most end of the catheter 310.

Additionally, the aorta has multiple branch vessels in addition to the renal arteries which effect the total flow in the aorta at a given location therein. Thus, a percentage of the blood flow that enters the abdominal aorta, i.e., past the diaphragm, is delivered in the normal rest state of circulation to the celiac trunk, the superior and inferior mesenteric arteries, and the renal arteries. Nonetheless, the flow segmentation created by the presence of the deployed catheter 310 is such that the blood flow in the outer blood flow stream 14 of a patient at rest is about 10% to about 90% of the total blood flow immediately up-stream of the up-stream or distal most end of the tubular member 316, i.e., of the total blood flow present in the section of the aorta 10 immediately adjacent to the renal arteries 12. Similarly, the blood flow in the inner blood flow stream 16 of a patient at rest is about 10% to about 90% of the total blood flow immediately up-stream of the up-stream or distal most end of the tubular member 316. The flow in the outer blood flow stream 14 is sufficient to provide adequate kidney function, although the flow required will vary depending upon factors such as the presence of drugs which increase flow or increase the ability of the tissue to withstand ischemic conditions.

While the renal arteries 12 are illustrated directly across from one another in FIG. 47, and the method is discussed primarily in terms of delivery of agent to both renal arteries together, it should be understood that the catheter may be positioned and used- to deliver agent to the renal arteries individually, and specifically in anatomies having the renal arteries longitudinally displaced from one another. When treatment of the renal arteries 12 is no longer needed, the flow of agent is stopped. The tubular member 316 is contracted by urging the pull line 326, distally, and the radially expandable member 318 is collapsed by removal of the inflation fluid, and the aortic flow diverter 310 is removed from the patient. A variety of suitable radially expandable tubular members 316 may be used in aortic flow diverter 310.

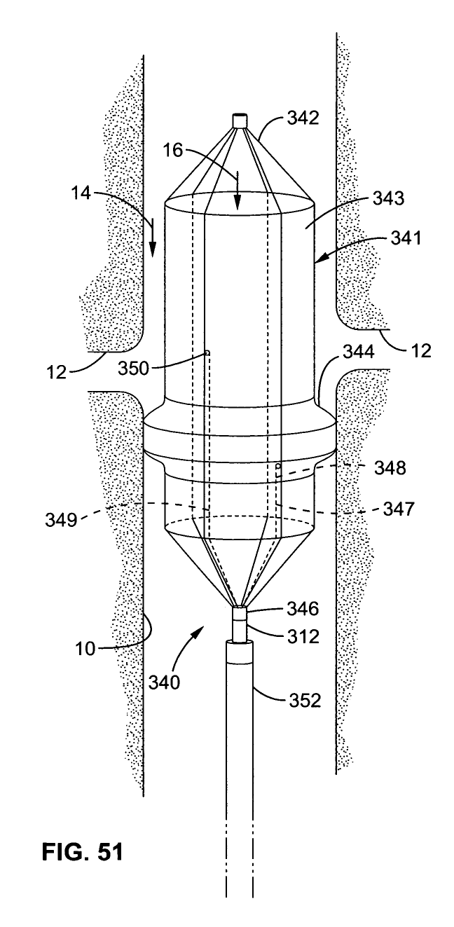

FIG. 51 illustrates another embodiment of an aortic flow diverter 340 in which the tubular member 341 comprises a self-expanding frame 342 having a sheath 343 thereon. As discussed above in relation to the embodiment of FIG. 45, catheter shaft 312 defines an inflation lumen 328 and an agent delivery lumen 332, and radially expandable member comprises a balloon 344 on an outer surface of sheath 343. For ease of illustration, the balloon 344 is shown as a transparent material. In the embodiment illustrated in FIG. 51, catheter shaft 312 comprises a multilumen proximal shaft 346 defining proximal sections of the inflation lumen 347 fluidly coupled to inflation port 348, and a second distal tubular member 349 fluidly coupled to agent delivery port 350. First tubular member 347 extends distally from the distal end of the proximal section of the inflation lumen 328 in the multilumen proximal shaft. Similarly, second tubular member 349 extends distally from the distal end of the proximal section of the agent delivery lumen 332 in the multilumen proximal shaft. First and second tubular members 347,349, are typically formed of thin-walled polymeric material such as polyimide, with an inner diameter of about 0.002 inch to about 0.006 inch, and a wall thickness of about 0.0005 inch to about 0.002 inch. In other embodiments, catheter shaft 312 comprises an outer tubular member with first and second inner tubular members defining inflation lumen and agent delivery lumen, respectively, extending within the outer member and out the distal end thereof. The agent delivery lumen 349 extends to a location proximal to the distal end of the tubular member 316 and distal to the balloon 344. One or more agent delivery ports 350 are provided in a distal section of the agent delivery lumens, as discussed above in relation to the embodiment of FIG. 45. In other embodiments, one or more additional agent delivery lumens may be provided.

In the embodiment illustrated in FIG. 51, the frame 342 comprises longitudinally extending filaments or struts, such as wires, joined together at the proximal and distal ends thereof. In another embodiment, frame 342 is formed of high strength metal, such as stainless steel, nickel-titanium alloy, or titanium. However a variety of suitable materials can be used including rigid polymers. The filaments typically have a round transverse cross section, with a diameter of about 0.006 inch to about 0.016 inch, or a rectangular transverse cross section with a thickness of about 0.001 inch to about 0.006 inch and a width of about 0.006 inch to about 0.016 inch. Sheath 343 is similar to sheath 322 discussed in relation to the embodiment of FIG. 45, and is preferably a thin walled elastomeric tubular member. The tubular member 341 is illustrated in FIG. 51 in the expanded configuration. The frame 342 is radially collapsible to a low profile configuration with the sheath 343 in a folded or pleated compact configuration for advancement within the patient's blood vessel. Once in place at a desired location within the blood vessel, a restraining member which applies a radially compressive force, which holds the frame in the collapsed smaller diameter configuration, is removed so that the frame expands. The frame may be held in the collapsed smaller diameter configuration by a variety of suitable restraining members such as a delivery catheter or removable outer sheath. For example, in one embodiment, the frame is deformed into the smaller diameter configuration within the lumen of a delivery catheter 352, and then expanded in the blood vessel lumen by longitudinally displacing the frame out the distal end of the delivery catheter 352 to thereby remove the radially compressive force of the delivery catheter 352. Although not illustrated, a pull line similar to pull line 326 discussed above in relation to the embodiment of FIG. 45 may be provided to apply additional radially expanding force to the filaments to supplement their inherent spring force, and is preferably provided in the embodiments having a radially expandable member comprising an inflatable balloon where inflation of the balloon creates a radially compressive force on the tubular member. In the embodiment illustrated in FIG. 51, balloon 344 is inflated into contact with the aorta wall 10 to an outer diameter which completely occludes the outer blood flow stream downstream of the renal arteries 12. Thus, the outer blood flow stream is directed into the branch vessels 12. However, the balloon may be configured to inflate to an outer diameter which does not completely occlude the downstream outer blood flow stream, as discussed above in relation to the embodiment of FIG. 47.

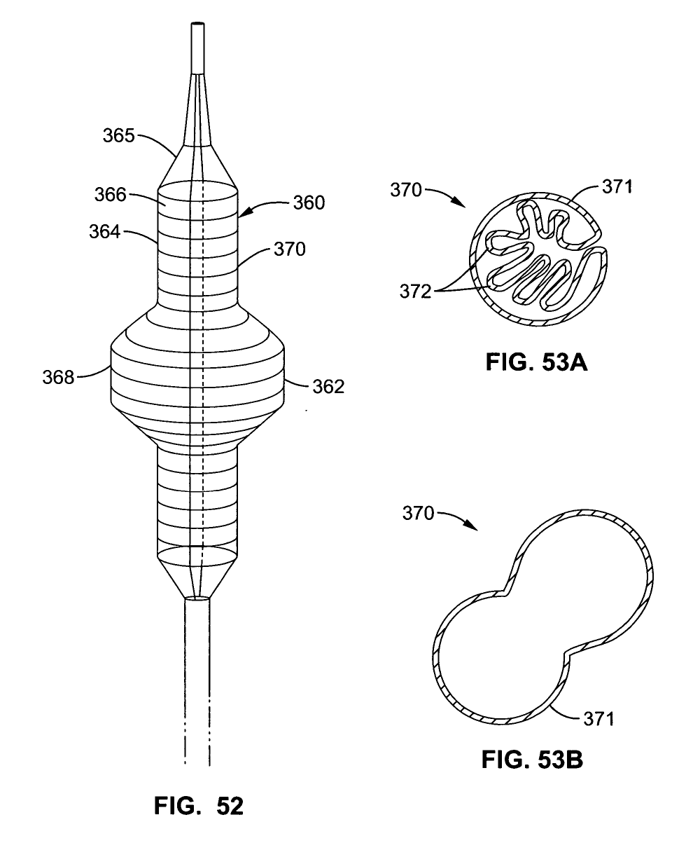

FIG. 52 illustrates another aortic flow diverter 360 sharing certain similarities with the aortic flow diverter 340 shown in FIG. 51 except that the balloon member is replaced with a radially enlarged section 362 of the tubular member 364. Thus, the frame 365, with sheath 366 thereon, forming the tubular member 364 does not have a uniform outer diameter, but instead radially expands from a collapsed configuration to define a smaller diameter section 367 defining tubular member 364, and a larger diameter section 368 defining a larger radial expandable member 362.

FIGS. 53A and 53B illustrate transverse cross sectional views of another tubular member 370 comprises a sheet configured to unwind from a wound low profile to an unwound radially expanded configuration, shown in FIG. 53B, to thereby radially expand the interior passageway of the tubular member 370. in FIG. 53A, the sheet 371 has a section wound back and forth into a plurality of folds 372. A restraining member (not shown) such as an outer sheath or delivery catheter is removed so that the sheet 371 unfolds as illustrated in FIG. 53B. The sheet section configured to be folded is preferably a thinner walled or otherwise more flexible than the section of the sheet which is not folded.

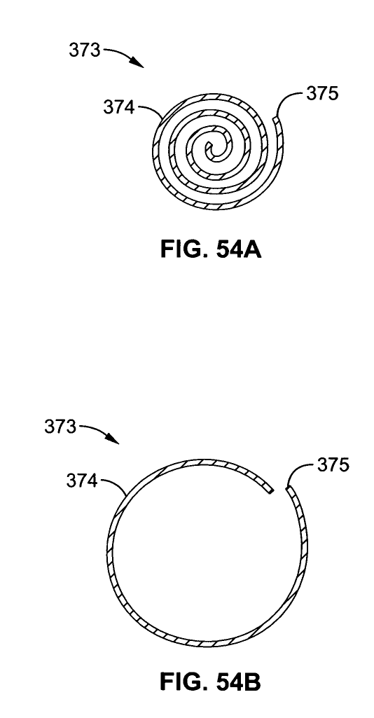

In another embodiment of a tubular member 373, illustrated in FIG. 54A and FIG. 54B, the sheet 374 is wound around itself into a rolled-up configuration having a free edge 375 extending the length of the sheet 374, which unrolls to the radially expanded configuration illustrated in FIG. 54B.

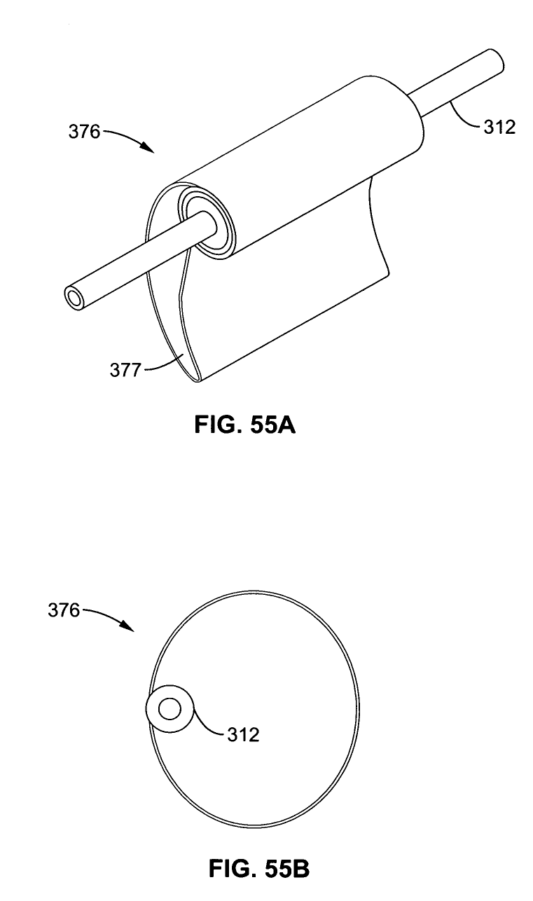

FIGS. 55A and 55B illustrate another tubular member 376 that is wound like a rolled awning type mechanism on support member 377 around shaft 312.

FIG. 55B illustrates tubular member 376 unwound from shaft 312. A variety of suitable unfurling or uncoiling configurations may be used in a tubular member which is radially expandable in accordance with the invention

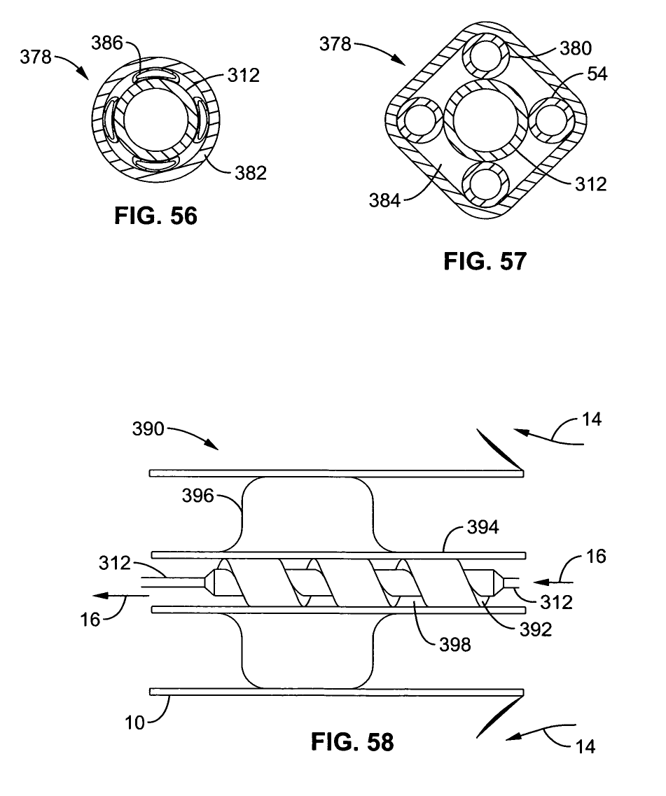

FIG. 56 illustrates a transverse cross sectional view of another tubular member 378 comprising a plurality of inflatable balloons 380 within an outer sheath 382. The balloons 380 can be inflated from a non-inflated low profile configuration to an inflated configuration shown in FIG. 57.

In the inflated configuration, shown in FIG. 57, inner passageway 384 is defined between the inflated balloons 380 in part by the sheath 382. Preferably, three or more balloons 380 are provided to in part define the inner passageway 380. Balloons 380 are preferably formed of a noncompliant material such as PET, or a compliant material such as polyethylene having reinforcing members such as wire members. Although four cylindrical balloons 378 are illustrated in FIG. 57, it should be understood that a variety of suitable configurations may be used, including balloons having outer channels such as a spiraled balloon defining an outer spirally extending blood flow channel, similar in many respects to perfusion balloons for dilatation. An inflation lumen is provided in the catheter shaft 312 in fluid communication with balloons 378.

FIG. 58 illustrates another embodiment of an aortic flow diverter 390 in an expanded state comprising an inner inflatable member 392 formed in a helical shape by either a blow molding process or by using helical wire constraints and attached to catheter 312. A cylindrical sheet member 394 encloses inner inflatable member 392. Outer annular inflatable member 396 is formed on the outside of cylindrical sheet member 394 and when inflated, occlude outer blood flow 14 in aorta 10. Inner blood flow 16 flows through helical passageway 398 formed by inner inflatable member 392. An infusion port (not shown) can be used to deliver fluid agent to outer blood flow 14 distal of the occlusion site of outer annular inflatable member 396.

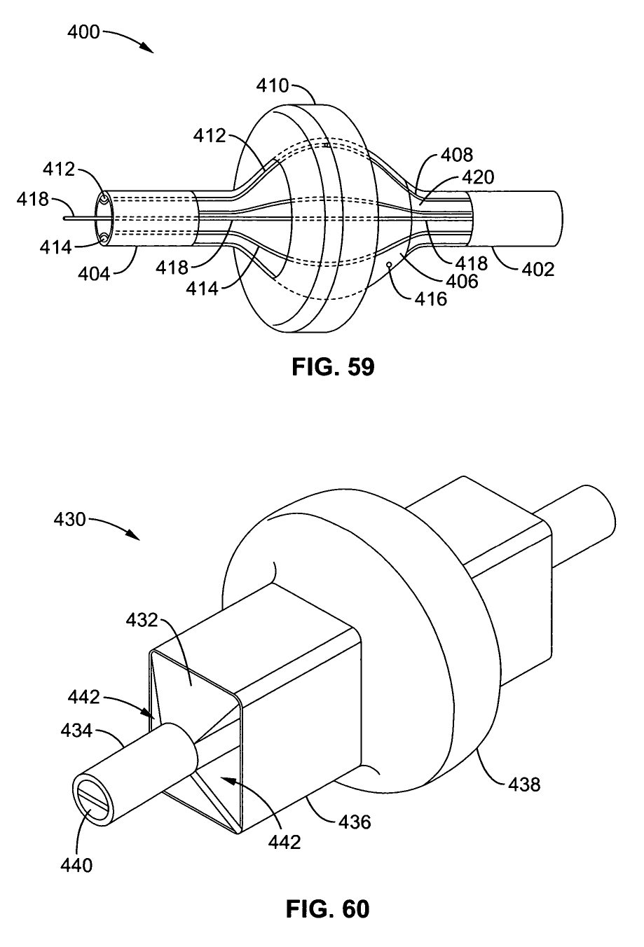

FIG. 59 Illustrates an aortic flow diverter 400 formed proximal of the distal section 402 of multilumen catheter 404. Tubular member 406 is supported on frame 408 consisting of three or more flexible legs connecting catheter 404 with catheter distal section 402. Flexible legs that comprise frame 408 may also be formed from longitudinal cuts into the flexible tubing forming catheter 404. Inflatable member 410 attaches to the exterior of tubular member 406 and is in fluid communication with an inflation lumen 412. Fluid agent lumen 414 in catheter 404 is connected to one or more flexible legs of frame 408 and fluidly connects to infusion port 416 through tubular member 406 and distal of inflatable member 410. Pull wire 418 is attached to the distal section 402 of catheter 404 and pulls the distal section 402 towards catheter 404 when retracted. During insertion, pull wire 418 is relaxed, frame 408 is in an extended state and inflatable member is collapsed and folded or pleated around frame 408. Aortic flow diverter 400 could also be encased in a delivery sheath (not shown) during insertion. When deployed, pull wire 418 is retracted causing frame 408 to expand against tubular member 406 and forming passageway 420. Inflatable member 410 is inflated to occlude or partially occlude blood flow as shown previously in FIG. 47. Fluid agent may be infused into an outer blood flow through infusion port 416 as shown previously in FIG. 47.

FIG. 60 illustrates another embodiment of an aortic flow diverter 430 with two inner inflatable tubular members 432, made of PET or other suitable compliant material, each formed to present a triangular cross section with one apex of the triangle attached to multilumen catheter 434 when inflated, and encased in cylindrical sheet material 436. Outer inflatable member 438, made of urethane, polyisoprene or other suitable material, encases cylindrical sheet material 436. Inner inflatable tubular members 432 are fluidly connected to an inflation lumen 440 in catheter 434 and fluidly connected to outer inflatable member 438. When inserted, aortic flow diverter 430 is deflated and outer inflatable member 438 pleated or folded around cylindrical sheet material 436. When place in an aorta or major blood vessel, inner inflatable tubular members 432 are inflated to form a blood flow passageway 442 with cylindrical sheet material 436. Outer inflatable member 438 inflates to occlude or partially occlude a major blood vessel as shown previously in FIG. 47.

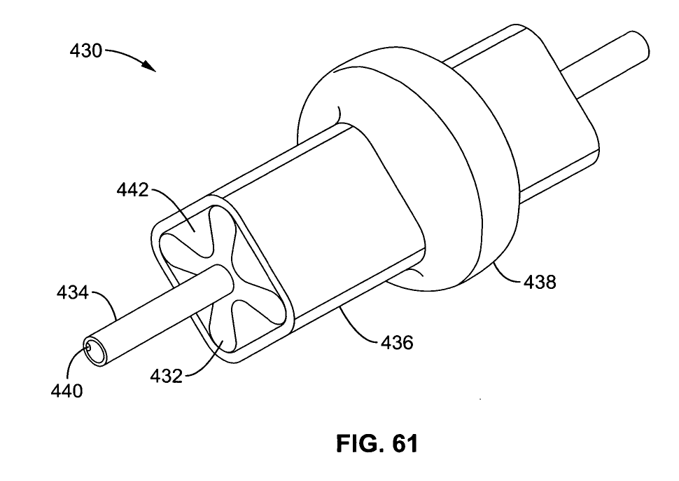

FIG. 61 illustrates another variation of the aortic flow diverter 430 in FIG. 60 where four inner inflatable tubular members 432 are formed to present a four lobed, clover shape, cross section within tubular member 436. Inner inflatable tubular members 432 are in fluid communication with outer inflatable member 438 and an inflation lumen 440 in multilumen catheter 434. Inner blood passageway 442 is formed between inner inflatable tubular members 432 and outer blood flow is occluded or partially occluded by outer inflatable member 438 as previously shown in FIG. 47.

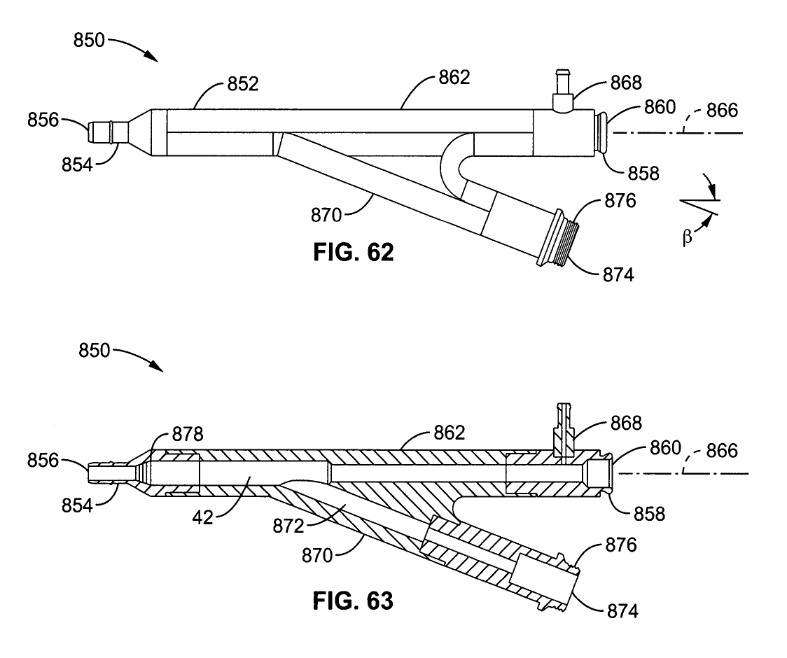

FIG. 62 through FIG. 65 illustrates an embodiment of a proximal coupler system 850 used to deploy and position renal fluid delivery devices adjunctive with interventional catheters. FIG. 62 and FIG. 63 illustrate a proximal coupler system 850 in side view, and cut away section view. Y Hub body 852 is configured with an introducer sheath fitting 854 at the distal end 856 of hub body 852 and a main adapter fitting 858 at the proximal end 860 of Y hub body 852. Main branch 862 has tubular main channel 864 aligned on axis 866. Main channel 862 fluidly connects introducer sheath fitting 854 and main adapter fitting 858. By way of example and not of limitation, one embodiment of main channel 864 is adapted to accommodate a 6 Fr guide catheter. Side port fitting 868 is positioned on main branch 862 and is fluidly connected to main channel 864. Secondary branch 870 has tubular branch channel 872 that intersects main channel 864 at predetermined transition angle β. In one beneficial embodiment, transition angle β is approximately 20 degrees. Proximal end 874 of secondary branch 870 has secondary fitting 876. In one beneficial embodiment, a channel restriction 878 is molded into introducer sheath fitting 854. Y hub body 852 may be molded in one piece or assembled from a plurality of pieces.

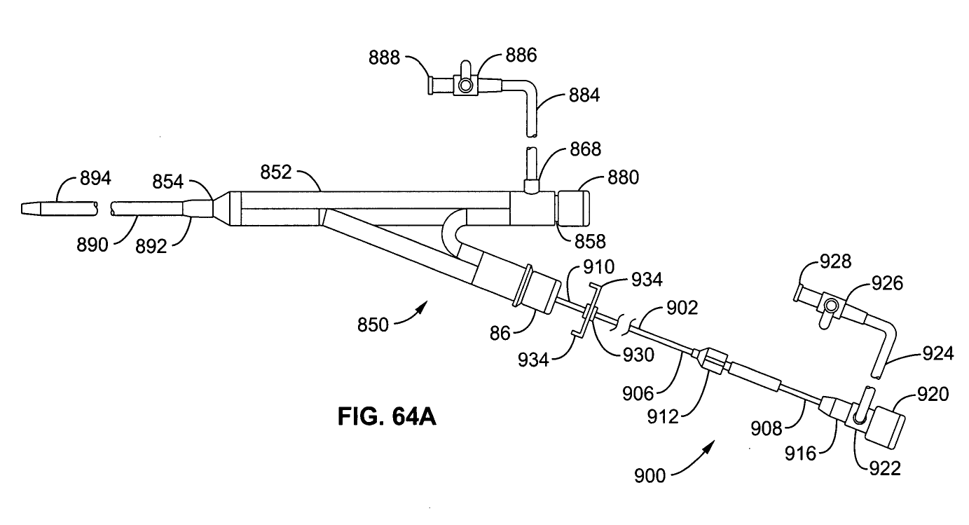

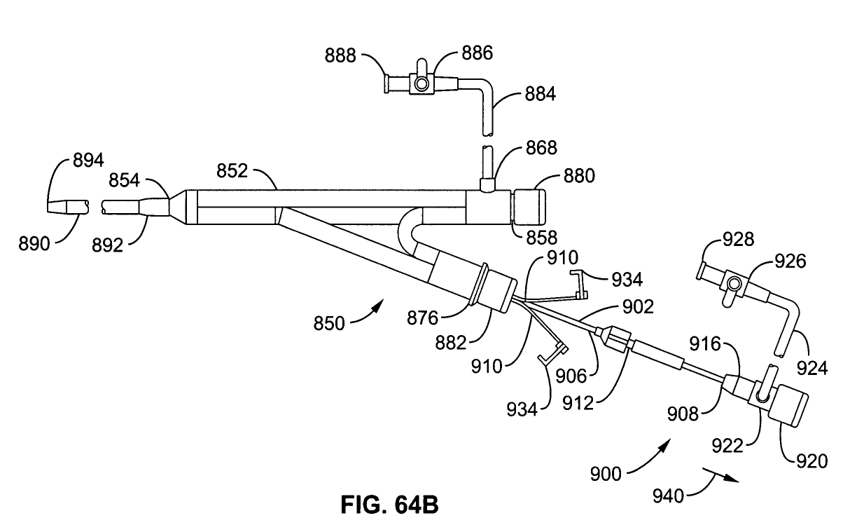

FIG. 64A and FIG. 64B illustrate a proximal coupler system 850 with a hemostasis valve 880 attached at main port 858 and Touhy Borst valve 882 attached at branch port 876. Fluid tube 884 is coupled to side port 868 and fluidly connects stop valve 886 and fluid port 888. Introducer sheath 890 with proximal end 892 and distal end 894 is coupled to Y hub body 852 at Sheath fitting 854. Proximal coupler system 850 is coupled to a local fluid delivery system 900. A stiff tube 902, has a distal end 904 (shown in FIG. 64B), a mid proximal section 906, and a proximal end 908. In one embodiment, stiff tube 902 is made of a Nickel-Titanium alloy. Stiff tube 902 is encased in delivery sheath 910 distal of mid proximal section 906. By way of example and not of limitation, delivery sheath 910 may be about 6 Fr to about 8 Fr in diameter. A torque handle 912 is coupled to stiff tube 902 at a mid proximal position 906. A material injection port 916 is positioned at the proximal end 908 of stiff tube 902. Material injection port 916 is coupled to an adapter valve 920 for introducing materials such as fluids. Side port fitting 922 is coupled to tube 924 and further coupled to stopcock 926 and fluid fitting 928. In an exemplary embodiment, adaptor 920 is a Luer valve. In another exemplary embodiment, side port fitting 922 is used for injecting a saline solution. Delivery sheath handle 930 is positioned and attached firmly at the proximal end 932 of delivery sheath 910. Delivery sheath handle 930 has two delivery handle tabs 934. In an exemplary embodiment, delivery sheath handle 930 is configured to break symmetrically in two parts when delivery handle tabs 934 are forced apart.

In FIG. 64B, Delivery sheath 910 is inserted through Touhy Borst adapter 882 through secondary branch channel 872 until distal end (not shown) of delivery sheath 910 is against channel restriction 878 (see FIG. 63). At that point, force 940 is applied in a distal direction at torque handle 912 to push stiff tube 902 through delivery tube 910. An aortic flow diverter (not shown) on distal end 904 of stiff tube 902 is adapted to advance distally into introduction sheath 890. In FIG. 64B, stiff tube 902 has been advanced into introduction sheath 890. In one mode, delivery sheath handle 930 is split in two by pressing inwardly on delivery handle tabs 934. Delivery sheath 910 is split by pulling delivery tabs 934 apart and retracted from Y hub assembly 852 through Touhy Borst adapter 882 to allow a medical intervention device (shown in FIG. 64) to enter hemostasis valve 880 for further advancement through main channel 864 (see FIG. 63) and adjacent to stiff tube 902. In a further mode, delivery sheath 910 is completely retracted from Y hub assembly 852 before splitting and removing from stiff tube 902.

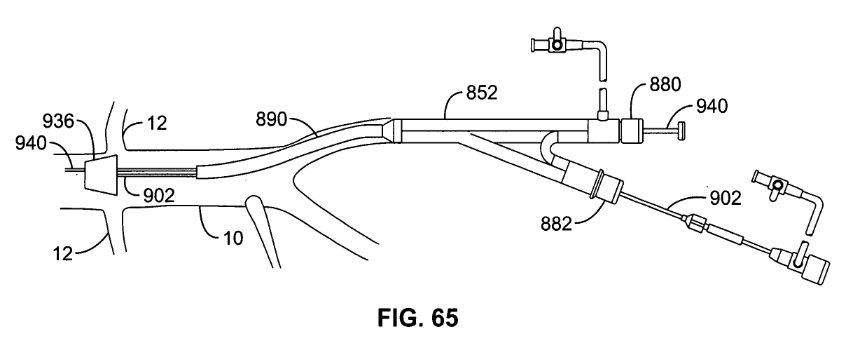

FIG. 65 is a stylized illustration of the proximal coupler system 850 of FIG. 64B with introducer sheath 890 is inserted in aorta system 10. Delivery sheath 910 (not shown) has been retracted proximally and removed and one or more fluid agent infusion devices 936 have been advanced through introducer sheath 809 and positioned near renal arteries 12. Intervention catheter 940 enters hemostasis valve 880 and is advanced through introducer sheath 890 and past aortic flow diverter 936 for further medical intervention while aortic flow diverter 936 remains in place at renal arteries 12. It is to be understood that proximal coupler systems can be further modified with additional branch ports to advance and position more than two devices through a single introducer sheath.

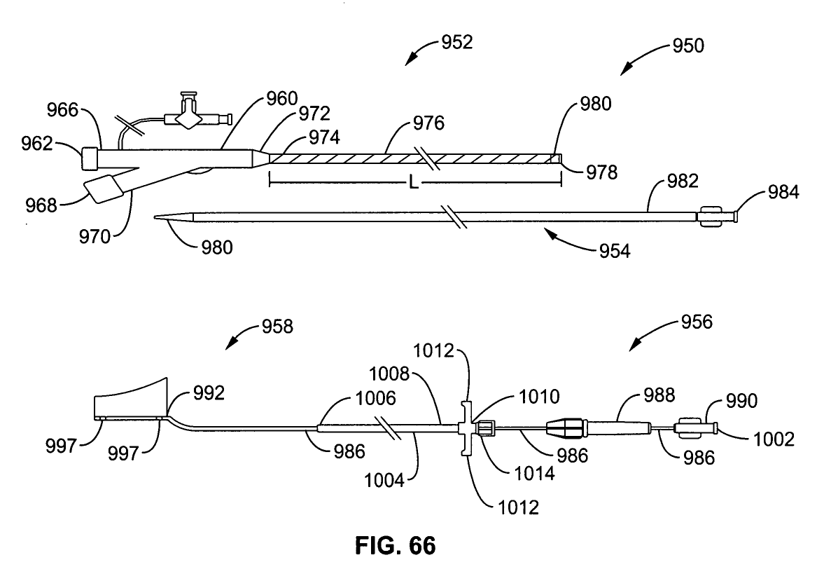

FIG. 66 illustrates a further embodiment of the proximal coupler assembly and fluid delivery assembly shown in FIG. 65. Renal therapy system 950 includes an introducer sheath system 952, a vessel dilator 954 and a fluid delivery system 956 with an aortic infusion assembly 958. Details of channels, saline systems and fittings as shown previously in FIG. 62 through FIG. 65 are omitted for clarity. Introducer sheath system 952 has Y hub body 960 as shown previously in FIG. 62 and FIG. 63 configured various inner structures as shown previously in FIG. 63. Y hub body 960 has hemostasis valve 962 on proximal end 966 and Touhy Borst valve 968 on secondary end 970. Distal end 972 of Y hub body 960 is coupled to proximal end 974 of introducer sheath 976. Introducer sheath 976 has distal tip 978 that has a truncated cone shape and radiopaque marker band 980. In one embodiment, introducer sheath 976 is constructed with an inner liner of PTFE material, an inner coiled wire reinforcement and an outer polymer jacket. Introducer sheath 976 has predetermined length L measured from proximal end 974 to distal tip 978.

Vessel dilator 954, with distal end 980 and proximal end 982 is a polymer, e.g. extrusion tubing with a center lumen for a guide wire (not shown). Distal end 980 is adapted with a taper cone shape. Proximal end 982 is coupled to a Luer fitting 984.

Fluid delivery system 956 has stiff tube 986, torque handle 988, and proximal hub 990 as previously described in FIG. 64A and FIG. 64B with aortic infusion assembly 958 coupled at distal end 992 with radiopaque marker bands 997 to aid positioning. The proximal hub 990 of fluid delivery system 956 has a Luer fitting 1002 for infusing a fluid agent, and is fluidly coupled with the stiff tube 986.

A single lumen, tear-away delivery sheath 1004 has a distal end 1006, a proximal end 1008, and slidingly encases stiff tube 986. delivery sheath 1004 is positioned between the torque handle 988 and the bifurcated catheter 956. The distal end 1006 has a shape and outer diameter adapted to mate with the channel restriction in the distal end of the main channel of the Y hub body as shown previously in FIG. 63. The proximal end 1008 of the delivery sheath 1004 is coupled to a handle assembly 1010 with two handles 1012 and a tear away cap 1014.

Dilator 954 is inserted through Touhy Borst valve 968 on secondary port 970 until distal end 980 protrudes from distal tip 978 of introducer sheath 976 to form a smooth outer conical shape. Distal tip 978 of introducer sheath 976 is positioned in the aorta system proximal of the renal arteries (not shown). Dilator 954 is removed and fluid delivery device 956 is prepared by sliding delivery sheath 1004 distally until aortic infusion assembly 958 is enclosed in delivery sheath 1004. Distal end 1006 of delivery sheath 1004 is inserted in Touhy Borst valve 968 and advanced to the restriction in the main channel of the Y hub body shown in FIG. 63. Aortic infusion assembly 958 is advanced distally into introducer sheath 976. Tear away delivery sheath 1004 is retracted and removed through Touhy Borst valve 968 as shown previously in FIG. 56B. Aortic infusion assembly 958 is advanced distally out of the distal tip 978 of introducer sheath 976 and positioned to infuse fluid agent in the renal arteries as shown in FIG. 65.

The various embodiments herein described for the present invention can be useful in treatments and therapies directed at the kidneys such as the prevention of radiocontrast nephropathy (RCN) from diagnostic treatments using iodinated contrast materials. As a prophylactic treatment method for patients undergoing interventional procedures that have been identified as being at elevated risk for developing RCN, a series of treatment schemes have been developed based upon local therapeutic agent delivery to the kidneys. Among the agents identified for such treatment are normal saline (NS) and the vasodilators papaverine (PAP) and fenoldopam mesylate (FM).

The approved use for fenoldopam is for the in-hospital intravenous treatment of hypertension when rapid, but quickly reversible, blood pressure lowering is needed. Fenoldopam causes dose-dependent renal vasodilation at systemic doses as low as approximately 0.01 mcg/kg/min through approximately 0.5 mcg/kg/min IV and it increases blood flow both to the renal cortex and to the renal medulla. Due to this physiology, fenoldopam may be utilized for protection of the kidneys from ischemic insults such as high-risk surgical procedures and contrast nephropathy. Dosing from approximately 0.01 to approximately 3.2 mcg/kg/min is considered suitable for most applications of the present embodiments, or about 0.005 to about 1.6 mcg/kg/min per renal artery (or per kidney). As before, it is likely beneficial in many instances to pick a starting dose and titrate up or down as required to determine a patient's maximum tolerated systemic dose. Recent data, however, suggest that about 0.2 mcg/kg/min of fenoldopam has greater efficacy than about 0.1 mcg/kg/min in preventing contrast nephropathy and this dose is preferred.

The dose level of normal saline delivered bilaterally to the renal arteries may be set empirically, or beneficially customized such that it is determined by titration. The catheter or infusion pump design may provide practical limitations to the amount of fluid that can be delivered; however, it would be desired to give as much as possible, and is contemplated that levels up to about 2 liters per hour (about 25 cc/kg/hr in an average about 180 lb patient) or about one liter or 12.5 cc/kg per hour per kidney may be beneficial.

Local dosing of papaverine of up to about 4 mg/min through the bilateral catheter, or up to about 2 mg/min has been demonstrated safety in animal studies, and local renal doses to the catheter of about 2 mg/min and about 3 mg/min have been shown to increase renal blood flow rates in human subjects, or about 1 mg/min to about 1.5 mg/min per artery or kidney. It is thus believed that local bilateral renal delivery of papaverine will help to reduce the risk of RCN in patients with pre-existing risk factors such as high baseline serum creatinine, diabetes mellitus, or other demonstration of compromised kidney function.

It is also contemplated according to further embodiments that a very low, systemic dose of papaverine may be given, either alone or in conjunction with other medical management such as for example saline loading, prior to the anticipated contrast insult. Such a dose may be on the order for example of between about 3 to about 14 mg/hr (based on bolus indications of approximately 10-40 mg about every 3 hours—papaverine is not generally dosed by weight). In an alternative embodiment, a dosing of 2-3 mg/min or 120-180 mg/hr. Again, in the context of local bilateral delivery, these are considered halved regarding the dose rates for each artery itself.

Notwithstanding the particular benefit of this dosing range for each of the aforementioned compounds, it is also believed that higher doses delivered locally would be safe. Titration is a further mechanism believed to provide the ability to test for tolerance to higher doses. In addition, it is contemplated that the described therapeutic doses can be delivered alone or in conjunction with systemic treatments such as intravenous saline.

It is to be understood that the invention can be practiced in other embodiments that may be highly beneficial and provide certain advantages. For example radiopaque markers are shown and described above for use with fluoroscopy to manipulate and position the introducer sheath and the aortic flow diverter. The required fluoroscopy equipment and auxiliary equipment is typically located in a specialized location limiting the in vivo use of the invention to that location. Other modalities for positioning aortic flow diverters are highly beneficial to overcome limitations of fluoroscopy. For example, non fluoroscopy guided technology is highly beneficial for use in operating rooms, intensive care units and emergency rooms. The use of non-fluoroscopy positioning allows aortic flow diverter systems and methods to be used to treat other diseases such as ATN and CHF.

In one embodiment, the aortic flow diverter is modified to incorporate marker bands with metals that are visible with ultrasound technology. The ultrasonic sensors are placed outside the body surface to obtain a view. In one variation, a portable, noninvasive ultrasound instrument is placed on the surface of the body and moved around to locate the device and location of both renal ostia. This technology is used to view the aorta, both renal ostia and the aortic flow diverter.

In another beneficial embodiment, ultrasound sensors are placed on the introducer sheath and the aortic flow diverter itself; specifically the distal end of the catheter. The aortic flow diverter with the ultrasonic sensors implemented allows the physician to move the sensors up and down the aorta to locate both renal ostia.

A further embodiment incorporates Doppler ultrasonography with the aortic flow diverter. Doppler ultrasonography detects the direction, velocity, and turbulence of blood flow. Since the renal arteries are isolated along the aorta, the resulting velocity and turbulence is used to locate both renal ostium. A further advantage of Doppler ultrasongoraphy is it is non invasive and uses no x rays.

A still further embodiment incorporates optical technology with the aortic flow diverter. An optical sensor is placed at the tip of the introducer sheath. The introducer sheath optical sensor allows visualization of the area around the tip of the introducer sheath to locate the renal ostia. In a further mode of this embodiment, a transparent balloon is positioned around the distal tip of the introducer sheath. The balloon is inflated to allow optical visual confirmation of renal ostium. The balloon allows for distance between the tip of the introducer sheath and optic sensor while separating aorta blood flow. That distance enhances the ability to visualize the image within the aorta. In a further mode, the balloon is adapted to allow profusion through the balloon wall while maintaining contact with the aorta wall. An advantage of allowing wall contact is the balloon can be inflated near the renal ostium to be visually seen with the optic sensor. In another mode, the optic sensor is placed at the distal tips of the aortic flow diverter. Once the aortic flow diverter is deployed within the aorta, the optic sensor allows visual confirmation of the walls of the aorta. The aortic flow diverter is tracked up and down the aorta until visual confirmation of the renal ostia is found. With the optic image provided by this mode, the physician can then track the aortic flow diverter to the renal arteries.

Another embodiment uses sensors that measure pressure, velocity or flow rate to located renal ostium without the requirement of fluoroscopy equipment. The sensors are positioned at the distal tip of the aortic flow diverter. The sensors display real time data about the pressure, velocity or flow rate. With the real time data provided, the physician locates both renal ostium by observing the sensor data when the aortic flow diverter is around the approximate location of the renal ostia. In a further mode of this embodiment, the aortic flow diverter has multiple sensors positioned at a mid distal and a mid proximal position on the catheter to obtain mid proximal and mid distal sensor data. From this real time data, the physician can observe a significant flow rate differential above and below the renal arteries and locate the approximate location. With the renal arteries being the only significant sized vessels within the region, the sensors would detect significant changes in any of the sensor parameters.

In a still further embodiment, chemical sensors are positioned on the aortic flow diverter to detect any change in blood chemistry that indicates to the physician the location of the renal ostia. Chemical sensors are positioned at multiple locations on the aortic flow diverter to detect chemical change from one sensor location to another.

The invention has been discussed in terms of certain preferred embodiments. One of skill in the art will recognize that various modifications may be made without departing from the scope of the invention. Although discussed primarily in terms of controlling blood flow to a branch vessel such as a renal artery of a blood vessel, it should be understood that the catheter of the invention could be used to deliver agent to branch vessels other than renal arteries, or to deliver to sites other than branch vessels, as for example where the catheter is used to deliver an agent to the wall defining the body lumen in which the catheter is positioned, such as a bile duct, ureter, and the like. Moreover, while certain features may be shown or discussed in relation to a particular embodiment, such individual features may be used on the various other embodiments of the invention.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A local renal infusion system for treating a renal system in a patient from a location within the abdominal aorta associated with abdominal aortic blood flow into first and second renal arteries via respective first and second renal ostia having unique relative locations along the abdominal aorta wall, comprising:
a local injection assembly;
a flow isolation assembly with a tubular wall having a longitudinal axis between a first end and a second end;
wherein the flow isolation assembly is adapted to be delivered to the location in a first condition with the tubular wall in a first configuration with a first diameter transverse to the longitudinal axis, and such that the first end is located upstream of the renal ostia and the second end is located downstream of the first end;
wherein the flow isolation assembly at the location is adjustable from the first condition to a second condition with the tubular wall in a second configuration;
wherein the tubular wall in the second configuration comprises a second diameter that is greater than the first diameter and that is substantially constant between the first and second ends such that a first region of abdominal aortic flow within an exterior flow path between the wall and the abdominal aortic wall is substantially isolated from a second region of abdominal aortic flow located within an interior flow path within the tubular wall, and further such that the first and second regions of abdominal aortic blood flow are not substantially diverted by the tubular shaped wall; and
wherein the local injection assembly is adapted to be fluidly coupled to a source of fluid agent located externally of the patient and to inject a volume of fluid agent from the source and into the first region between the abdominal aortic wall and the tubular wall in the second configuration at the location.

2. The system of claim 1, wherein:
the local injection assembly comprises first and second injection ports;
wherein the first and second injection ports are adapted to be delivered to first and second unique respective positions that are fluidly coupled to the first region at the location;
wherein the first and second injection ports at the first and second positions, respectively, are adapted to be fluidly coupled to the source of fluid agent located externally of the patient; and
wherein the first and second injection ports are adapted to simultaneously inject a volume of fluid agent from the source and principally into the first and second renal arteries, respectively, via their respective renal ostia from the first and second positions, also respectively.

3. The system of claim 2, wherein the first region comprises an outer region of abdominal aortic blood flow generally along the abdominal aorta wall at the location.

4. The system of claim 3, wherein the first and second positions are adapted to inject the volume of fluid agent into first and second separate portions of the abdominal aortic flow within the outer region.

5. The system of claim 3, wherein the first and second positions are located at least 90 degrees apart relative to a circumference around a longitudinal axis of the abdominal aorta at the location.

6. The system of claim 3, wherein the first and second positions are located about 120 degrees apart relative to the circumference.

7. The system of claim 3, wherein the first and second positions are located about 180 degrees apart relative to the circumference.

8. The system of claim 1, further comprising:
a flow isolation assembly;
wherein the flow isolation assembly is adjustable between a first condition and a second condition;
wherein the flow isolation assembly in the first condition is adapted to be delivered to the location;
wherein the flow isolation assembly at the location is adjustable from the first condition to the second condition that is adapted to substantially isolate fluid communication between the first region and a second region of abdominal aortic blood flow;
wherein the local injection assembly in the second configuration is adapted to cooperate with the flow isolation assembly in the second condition so as to inject the volume of fluid agent from the source and into the first region such that the injected volume of fluid agent flows substantially into the first and second renal arteries, respectively, via the respective first and second renal ostia.

9. The system of claim 8, wherein:
the flow isolation assembly comprises a wall that is adjustable between first and second configurations corresponding with the first and second conditions, respectively;
in the first configuration the wall is adapted to be delivered to the location;
in the second configuration the wall is adapted to substantially isolate the first region of abdominal aortic blood flow from the second region of abdominal aortic blood flow.

10. The system of claim 9, wherein:
the wall comprises a tubular wall with a first end, a second end, an outer surface, and an inner surface that defines a longitudinal passageway along a longitudinal axis between the first and second ends;
in the first configuration the longitudinal passageway comprises a first inner diameter;
in the second configuration the longitudinal passageway comprises a second inner diameter that is greater than the first inner diameter;
the first region comprises an exterior flow path around the outer surface between the tubular wall and the abdominal aortic wall; and
the second region comprises an interior flow path within the tubular wall and along the longitudinal passageway.

11. The system of claim 10, wherein:
the flow isolation assembly further comprises a support member that is substantially ring-shaped and is coupled to the tubular wall at one of the first and second ends;
wherein in the first configuration the support member is in a radially collapsed condition with a collapsed diameter transverse to the longitudinal axis of the tubular wall;
wherein in the second configuration the support member is in a radially extended condition with an extended diameter that is greater than the collapsed diameter;
wherein the support member in the radially extended condition supports the tubular wall in an expanded tubular shape at least at the one end.

12. The system of claim 11, wherein:
the support member comprises a superelastic metallic wire with two opposite ends and a curved region between the two opposite ends around a circumferential path;
the wire has a memory shape with the two opposite ends at first and second memory positions relative to each other with respect to the circumferential path such that the curved region has a memory diameter that is less than the extended diameter; and
the wire is secured relative to the tubular member in a superelastically deformed condition with the two opposite ends at first and second displaced positions relative to each other such that the support member in the second configuration and with the extended diameter comprises a superelastically deformed condition for the wire.

13. The system of claim 11, wherein the support member comprises a first support member, the collapsed diameter is a first collapsed diameter, and the extended diameter is a first extended diameter, and further comprising:
a second support member that is substantially ring-shaped and is coupled to the tubular wall at the other of the first and second ends;
wherein in the first configuration the second support member is in a radially collapsed condition with a second collapsed diameter transverse to the longitudinal axis;
wherein in the second configuration the second support member is in a radially extended condition with a second extended diameter that is greater than the second collapsed diameter;
wherein the second support member in the radially extended condition supports the tubular wall in a tubular shape at least at the other end.

14. The system of claim 13, further comprising:
a longitudinal spine extending between the first and second support members.

15. The system of claim 14, wherein:
the local injection assembly comprises an injection port located along the longitudinal spline and oriented so as to inject the volume of fluid agent into the first region.

16. The system of claim 14, wherein:
the local injection assembly comprises first and second injection ports located at first and second respective positions on opposite sides transverse to the longitudinal spine and that are adapted to inject the volume of fluid agent in first and second opposite directions transverse to the longitudinal spine.

17. The system of claim 1, wherein the tubular wall comprises a sheet of polytetrafluoroethylene (PTFE) material that is formed in a substantially tubular shape.

18. The system of claim 10, wherein:
the first end of the tubular wall in the second configuration has a first inner diameter;
the second end of the tubular wall in the second configuration has a second inner diameter; and
the first and second inner diameters are different such that the tubular wall comprises a frustroconical shape.

19. The system of claim 12, wherein the superelastic metallic wire comprises a nickel-titanium alloy.

20. The system of claim 1, wherein the retraction member comprises a wire.

21. The system of claim 1, wherein the retraction member comprises a thread.

22. The system of claim 1, further comprising:
an elongate body with a proximal end portion, a distal end portion that is adapted to be positioned at the location with the proximal end portion extending externally of the patient, a first body passageway, and a second body passageway;
a proximal hub assembly with a housing that comprises a first branch passageway and a second branch passageway;
wherein the first branch passageway is coupled to the first body passageway and is adapted to couple to the source of fluid agent externally of the patient;
wherein the first body passageway is coupled to an injection port and forms at least in part the local injection assembly; and
wherein the second branch passageway is coupled to the second body passageway; and
wherein the proximal end portion of the retraction member extends within the second branch passageway, and the distal end portion of the retraction member extends along the second body passageway.

23. The system of claim 1, wherein the distal end portion of the retraction member is coupled to the second end of the tubular wall.

24. The system of claim 23, further comprising:
an elongate body with a proximal end portion and a distal end portion;
a pulley located along the distal end portion;
wherein the tubular wall is located along the distal end portion with the first end located distally from the second end;
wherein the pulley is located distally adjacent to the first end of the tubular wall;
wherein the distal end portion of the retraction member is adapted to extend across the second end and distally beyond the first end of the tubular wall and loop around the pulley over the first end and proximally along and externally of the tubular wall where it is coupled to the second end; and
wherein the distal end portion of the retraction member is adapted to pull the second end of the tubular wall toward the first end of the tubular wall upon proximal retraction of the proximal end portion of the retraction member externally of the patient.

25. The system of claim 23, further comprising:
an elongate body with a proximal end portion and a distal end portion that is adapted to be positioned at the location while the proximal end portion extends externally of the patient;
a circumferential passageway within the tubular wall at the second end;
a ring-shaped support member located within the circumferential passageway;
wherein the tubular wall is located along the distal end portion with the first end located distally of the second end; and
wherein the distal end portion of the retraction member extends along the distal end portion of the elongate body and externally therefrom adjacent to the second end; and
wherein the distal end portion of the retraction member is coupled to support member through an opening in the circumferential passageway and is adapted to retract the support member distally from the second end.

26. The system of claim 23, further comprising:
an elongate body with a proximal end portion and a distal end portion that is adapted to be positioned at the location while the proximal end portion extends externally of the patient;
wherein the tubular wall is located along the distal end portion with the first end located distally of the second end; and
wherein the distal end portion of the retraction member extends along the distal end portion of the elongate body and externally therefrom proximally adjacent to the second end; and
wherein the distal end portion of the retraction member is adapted to retract the second end proximally from the first end upon proximal retraction of the proximal end portion of the retraction member externally of the patient.

27. The system of claim 1, wherein the expandable member is inflatable.

28. The system of claim 27, wherein the expandable member and inflatable member are inflatable together.

29. The system of claim 1, wherein:
the inflatable member in the inflated condition has a first shape;
the tubular wall in the second configuration has a second shape relative to the longitudinal passageway; and
the at least one flow passageway is formed at gaps between the first shape of the inflatable member and the second shape of the tubular wall.

30. The system of claim 29, wherein a plurality of discrete flow passageways are formed along the longitudinal passageway between the first and second ends with the inflatable member in the inflated condition and the tubular wall in the second configuration.

31. The system of claim 1, wherein:
the wall assembly comprises a tubular member coupled to the plurality of arms and that is constructed of a substantially stretchable material; and
the substantially stretchable material is stretched in the radially extended condition relative to the radially collapsed condition.

32. The system of claim 31, wherein:
the elongate body comprises an outer member that is tubular along the longitudinal axis with a proximal end portion and a distal end portion with a distal tip, and an inner member with a proximal end portion and a distal end portion and a distal tip;
the inner member is located within the outer member;
the inner and outer members are moveable longitudinally relative to each other;
the distal end portion of the inner member extends distally from the distal end portion of the outer member such that the distal tip of the inner member is located distally from the distal tip of the outer member;
the proximal positions for the arms are located along the distal end portion of the outer member;
the distal positions for the arms are located along the distal end portion of the inner member;
in the first condition the distal tips of the outer and inner members have first relative positions relative to each other;
in the second condition the distal tips of the outer and inner members have second relative positions relative to each other and that are longitudinally collapsed relative to the first condition; and
the longitudinal collapse of the distal tips from the first condition to the second condition forces the arms to bias radially outward from the elongate body into the radially extended condition.

33. The system of claim 1, wherein:
the vent comprises an aperture through the wall.

34. The system of claim 1, wherein:
the vent comprises a plurality of apertures through the wall.

35. The system of claim 1, wherein:
the first portion is adapted to be positioned along the location so as to correspond at least in part with the respective locations of the renal ostia; and
the second portion is adapted to be positioned downstream from the renal ostia along the location.

36. The system of claim 35, wherein:
the wall comprises a tubular wall with a first end, a second end, an outer surface, and an inner surface that defines a longitudinal passageway between the first and second ends;
in the first configuration the longitudinal passageway comprises a first inner diameter;
in the second configuration the longitudinal passageway comprises a second inner diameter that is greater than the first inner diameter and is tapered between the first end and the second end;
in the second configuration the first end is located upstream from the renal ostia and the second end is located downstream of the renal ostia;
the first region comprises an exterior flow path around the outer surface between the tubular wall and the abdominal aortic wall;
the second region comprises an interior flow path within the tubular wall and along the longitudinal passageway; and
the second portion with the vent is located adjacent the second end.

37. The system of claim 36, wherein:
the second inner diameter has a reducing taper from the second end to the first end.

38. The system of claim 36, wherein:
the second inner diameter has a reducing taper from the first end to the second end.

39. The system of claim 36, wherein:
the vent comprises a plurality of apertures through the tubular wall and spaced around a circumference around the longitudinal passageway.

40. The system of claim 1, wherein
the wall comprises a tubular wall with an outer surface and an inner surface that defines a longitudinal passageway between the first and second ends;
in the first configuration the longitudinal passageway comprises a first inner diameter;
in the second configuration the longitudinal passageway comprises a second inner diameter that is greater than the first inner diameter and has a reducing taper from the first end to the second end;
the first region comprises an exterior flow path around the outer surface between the tubular wall and the abdominal aortic wall; and
the second region comprises an interior flow path within the tubular wall and along the longitudinal passageway.

41. The system of claim 1, wherein:
the flow isolation assembly comprises a wall that extends between a first end and a second end, a longitudinal spine with a longitudinal axis, and a support member that is substantially ring-shaped along a circumference surrounding the longitudinal axis;
in the first condition the support member is in a radially collapsed condition with a collapsed diameter transverse to the longitudinal axis;
in the second condition the support member is in a radially extended condition with an extended diameter transverse to the longitudinal axis and that is greater than the collapsed diameter;
the first end of the wall is secured at a first location along the longitudinal spine;
the support member is secured to the longitudinal spine at a second location that is spaced from the first location;
the second end of the wall is secured to the support member;
the wall has an arced portion that extends between the first and second locations from only a first portion of the circumference of the support member that is less than the whole circumference of the support member;
in the second condition at the location the first end is located upstream from the renal ostia and the second end is located downstream from the first end;
the first region is located between the wall extending between the support member at the first location and the second location along the longitudinal spine; and
the portion of the outer region of abdominal aortic blood flow that is not included in the first region corresponds with a second portion of the circumference of the support member and from which the wall does not extend toward the second location.

42. The system of claim 41, wherein:
the support member comprises a first support member, the collapsed diameter is a first collapsed diameter, the extended diameter is a first extended diameter;
the flow isolation assembly further comprises a second support member that is substantially ring-shaped along a circumference surrounding the longitudinal axis;
in the first condition the second support member is in a radially collapsed condition with a second collapsed diameter transverse to the longitudinal axis;
in the second condition the second support member is in a radially extended condition with a second extended diameter transverse to the longitudinal axis and that is greater than the second collapsed diameter;
the second support member is secured to the longitudinal spine at the first location;
the first end of the wall is secured to the second support member;
the wall extends between the first and second support members from only a portion of the circumference of the second support member that is less than the whole circumference of the second support member; and
the first region is located between the wall extending between the first and second support members.

43. The system of claim 1, wherein:
the first region comprises first and second portions of the outer region that are spaced from each other around the circumference of the abdominal aortic wall.

44. The system of claim 43, wherein:
the local injection assembly comprises first and second injection members and first and second injection ports located on the first and second injection members, respectively;
the flow isolation assembly comprises first and second walls positioned along the first and second injection members, respectively;
the first and second injection members are adjustable to a radially collapsed orientation relative to each other such that the local injection assembly is adapted to be delivered to the location through a lumen of an introducer sheath;
the first and second injection members at the location are adjustable from the radially collapsed orientation to a radially extended orientation wherein they are spaced transverse to a longitudinal axis by a distance such that the first and second injection ports are adapted to be positioned at first and second unique relative positions within the first and second portions, respectively, within the first region at the location;
the first and second walls in the first condition have first configurations, respectively, that are collapsed relative to the first and second injection members, respectively;
the first and second walls in the second condition have extended configurations, respectively, that are extended with a shape relative to the first and second injection members, respectively;
in the respective extended configurations the first and second walls are adapted to substantially isolate the first and second portions, respectively, of the first region from the second region of the abdominal aorta flow; and
the first and second injection ports at the first and second positions are adapted to be fluidly coupled to the source of fluid agent located externally of the patient and to inject a volume of fluid agent from the source and into the first and second portions, respectively, that are isolated from the second region of abdominal aortic flow by the first and second walls, also respectively.

45. The system of claim 43, wherein:
the flow isolation assembly further comprises first and second support members secured to the first and second injection members, respectively;
each support member is secured to the respective injection member at a first location;
the first and second walls are secured to the first and second support members, respectively;
the first and second support members are adapted to impart the shape to the first and second walls in the respectively extended configurations.

46. The system of claim 43, wherein:
the shape of each wall in the extended configuration comprises a partial funnel shape; and
the first and second injection ports are adapted to be fluidly coupled to a region defined by the partial funnel shape.

47. The system of claim 1, wherein:
the flow isolation assembly comprises a tubular wall with a first end, a second end, and a longitudinal passageway extending between the first and second ends;
the local injection assembly further comprises a passageway within the tubular wall with a common channel fluidly coupled to first and second branch channels;
the first and second injection ports are located along the tubular wall;
the common channel is adapted to be fluidly coupled to the source of fluid agent located externally of the patient when the flow isolation assembly is in the second condition at the location; and
the first and second branch channels are fluidly coupled to the first and second injection ports.

48. The system of claim 47, wherein the first and second injection ports comprises apertures through the tubular wall and into the first and second branch channels, respectively.

49. The system of claim 1, further comprising:
at least one marker coupled to the local injection assembly or the flow isolation and that is adapted to indicate to an operator externally of the patient the location of the local injection assembly or flow isolation assembly, respectively, at the location within the patient.

50. The system of claim 49, wherein the at least one marker comprises a radiopaque marker.

51. The system of claim 1, further comprising:
a proximal coupler assembly that is adapted to be fluidly coupled to a source of fluid agent externally of the patient, and also to the local injection assembly at the location within the patient.

52. The system of claim 1, further comprising:
a source of fluid agent that is adapted to be coupled to the local injection assembly.

53. The system of claim 52, wherein the fluid agent comprises a renal protective agent, diuretic, Furosemide or an analog or derivative thereof, Thiazide or an analog or derivative thereof, a vasopressor, Dopamine or an analog or derivative thereof, a vasodilator, a vasoactive agent, Papaverine or an analog or derivative thereof, a Calcium-channel blocker, Nifedipine or an analog or derivative thereof, Verapamil or an analog or derivative thereof, fenoldapam mesylate or an analog or derivative thereof, or a dopamine DA1 agonist.

54. The system of claim 1, further comprising:
a vascular access system with an elongate tubular body with at least one lumen extending between a proximal port and a distal port that is adapted to be positioned within a vessel having translumenal access to the location;
a percutaneous translumenal interventional device that is adapted to be delivered to an intervention location across the location while the local injection assembly and renal flow assembly are at the location; and
wherein the local injection assembly, renal flow assembly, and percutaneous translumenal interventional device are adapted to be delivered percutaneously to the location and intervention location, respectively, through the vascular access device.

55. The system of claim 54, wherein the percutaneous translumenal interventional device comprises an angiographic catheter.

56. The system of claim 54, wherein the percutaneous translumenal interventional device comprises a guiding catheter.

57. The system of claim 54, wherein the percutaneous translumenal interventional device is between about 4 French and about 8 French.

58. The system of claim 1, wherein:
the local injection assembly comprises first and second injection ports;
the flow isolation assembly comprises a spine with a longitudinal axis, a radial support member coupled to the spine at a first location along the longitudinal axis, and an adjustable wall comprising a sheet of substantially flexible material;
wherein the adjustable wall has a first end that is coupled to the radial support member at the first location and a second end that is coupled to the spine at a second location that is spaced along the longitudinal axis from the first location;
wherein the radial support member is adjustable from a first configuration to a second configuration that characterize at least in part the first and second conditions;
in the first configuration the radial support member is radially collapsed relative to the longitudinal axis and positions the adjustable wall in a radially collapsed condition relative to the longitudinal axis, wherein the radial support member and adjustable wall are adapted to be delivered with the spine to the location;
the radial support member at the location is adjustable to the second configuration that is radially extended from the longitudinal axis relative to the first configuration and that at least in part positions the adjustable wall in a radially extended condition that is radially extended from the longitudinal axis relative to the radially collapsed condition;
said adjustable wall in the radially extended condition being positionable along the location relative to the first and second injection ports so as to isolate the injected volume of fluid agent to flow substantially within the first region along the location.

59. The system of claim 58, wherein:
the radial support member comprises a ring-shaped member adjustable between a first shape in the first configuration and a second shape in the second configuration;
the first shape has a first diameter; and
the second shape has a second diameter that is larger than the first diameter and is adapted to radially engage the abdominal aortic wall at an anchoring location along the location within the abdominal aorta so as to anchor the radial support member at the anchoring location.

60. The system of claim 59, wherein the radial support member comprises a ring that is constructed from superelastic material.

61. The system of claim 60, wherein the superelastic material comprises a nickel-titanium alloy.

62. The system of claim 67, wherein:
the ring-shaped member has a circumference; and
the adjustable wall is coupled to the ring-shaped member along only a part of the circumference that is less than all of the circumference of the ring-shaped member.

63. The system of claim 67, wherein:
the part of the circumference of the ring-shaped member in the second shape is adapted to substantially correspond with the first region of the outer region.

64. The system of claim 58, wherein the radial support member comprises a first radial support member, and further comprising:
a second radial support member secured to the spine at the second location;
wherein the second radial support member is adjustable between first and second configurations that together with the first and second configurations for the first radial support member characterize first and second conditions for the flow isolation assembly;
in the first configuration the second radial support member is radially collapsed relative to the longitudinal axis and at least in part positions the adjustable wall to the first condition that is radially collapsed relative to the longitudinal axis, wherein the second radial support member and adjustable wall are adapted to be delivered with the spine to the location;
the second radial support member at the location is adjustable to the second configuration that is radially extended from the longitudinal axis relative to the first configuration and that at least in part positions the adjustable wall in the second condition that is radially extended from the longitudinal axis relative to the first condition.

65. The system of claim 58, wherein:
the adjustable wall in the second condition is adapted to divert an interior region of flow along the abdominal aorta located within the outer region to flow toward the first region; and
the diverted region of abdominal aortic flow is adapted to flow substantially into the first and second renal ostia.

66. The system of claim 58, wherein:
the spine between the first and second locations has a shape in the second configuration that is adapted to position the second location a distance away from the abdominal aorta wall.

67. The system of claim 66, wherein the distance extends beyond the outer region and into an inner region of abdominal aortic blood flow.

68. A proximal coupler assembly for concurrent use with a bilateral local renal delivery device and percutaneous translumenal interventional device, wherein the bilateral local renal delivery device comprises an elongate body with a proximal end portion and a distal end portion and a local injection assembly located along the distal end portion, the assembly comprising:
a housing with a distal end and a proximal end;
wherein the distal end comprises a distal coupler that is adapted to be coupled to an introducer sheath that provides percutaneous translumenal access into a vasculature of a patient that leads to a location within an abdominal aorta associated with renal artery ostia;
wherein the proximal end comprises an adjustable hemostatic coupler;
wherein the adjustable hemostatic coupler is adapted to simultaneously receive the bilateral local renal delivery device and the percutaneous translumenal device into the housing and is substantially aligned along a longitudinal axis with the distal end of the housing;
means for securing the proximal end portion of the bilateral local renal delivery device off-axis relative to the longitudinal axis so as to reduce interference between the percutaneous translumenal interventional device and the bilateral local renal delivery device when the percutaneous translumenal interventional device is manipulated within the hemostatic valve.

69. The assembly of claim 68, further comprising:
means for flushing the housing with a volume of fluid when the bilateral local renal delivery device and percutaneous translumenal interventional device are located within the housing through the adjustable hemostatic coupler.
